SEQUENCE LISTING (55 sequences)

SEQ ID NO: 1 - protein, 5 aa, synthetic construct
GYYWN

SEQ ID NO: 2 - protein, 16 aa, synthetic construct
EINHSGSTNYNPSLKS

SEQ ID NO: 3 - protein, 9 aa, synthetic construct
NLYNWNLDS

SEQ ID NO: 4 - protein, 11 aa, synthetic construct
RASQGIRNDLG

SEQ ID NO: 5 - protein, 7 aa, synthetic construct
VASSLQS

SEQ ID NO: 6 - protein, 9 aa, synthetic construct
LQHNSYPRT

SEQ ID NO: 7 - protein, 5 aa, synthetic construct
GYYWN

SEQ ID NO: 8 - protein, 16 aa, synthetic construct
EINHSGSTSYNPSLKS

SEQ ID NO: 9 - protein, 9 aa, synthetic construct
VLYNWNVDS

SEQ ID NO: 10 - protein, 11 aa, synthetic construct
RASQDIRNDVG

SEQ ID NO: 11 - protein, 7 aa, synthetic construct
AASRLQS

SEQ ID NO: 12 - protein, 9 aa, synthetic construct
LQHNSYPRT

SEQ ID NO: 13 - protein, 7 aa, synthetic construct
SGGYYWS

SEQ ID NO: 14 - protein, 16 aa, synthetic construct
NIYYSGSTYYNPSLKS

SEQ ID NO: 15 - protein, 20 aa, synthetic construct
EKALATVTPSGYENYYTVDV

SEQ ID NO: 16 - protein, 11 aa, synthetic construct
WASQGISSYLA

SEQ ID NO: 17 - protein, 7 aa, synthetic construct
AASTLQS

SEQ ID NO: 18 - protein, 9 aa, synthetic construct
QHLNSYPLT

SEQ ID NO: 19 - protein, 7 aa, synthetic construct
SGGYYWS

SEQ ID NO: 20 - protein, 16 aa, synthetic construct
NIYYSGSTYYNPSLKS

SEQ ID NO: 21 - protein, 20 aa, synthetic construct
EKALASVIPSGYENYYVVDV

SEQ ID NO: 22 - protein, 11 aa, synthetic construct
WASQGIAGYLA

SEQ ID NO: 23 - protein, 7 aa, synthetic construct
AASTLQS

SEQ ID NO: 24 - protein, 9 aa, synthetic construct
QHLNSYPLT

SEQ ID NO: 25 - protein, 5 aa, synthetic construct
DYYMS

SEQ ID NO: 26 - protein, 17 aa, synthetic construct
YISSSGSTIYYTDSVKG

SEQ ID NO: 27 - protein, 7 aa, synthetic construct
DGVGFDY

SEQ ID NO: 28 - protein, 12 aa, synthetic construct
RASQSVSSSYLA

SEQ ID NO: 29 - protein, 7 aa, synthetic construct
AASSRAT

SEQ ID NO: 30 - protein, 10 aa, synthetic construct
QQYGSSPLYT

SEQ ID NO: 31 - protein, 117 aa, synthetic construct
QVQLQQWGAGLLKPSETLSLTCAVYGGSFSGYYWNWIRQPPGKGLEWIGEINHSGSTNYNPSLKSRVTILVDTSKNQFSLKLSSVTAADTAVYYCARNLYNWNLDSWGQGTLVTVSS

SEQ ID NO: 32 - protein, 107 aa, synthetic construct
DIQMTQSPSSLSASVGDRVTITCRASQGIRNDLGWYQQKPGRAPKRLIYVASSLQSGVPSRFSGSGSGTEFTLTISSLQPEDFATYYCLQHNSYPRTFGQGTKVEIK

SEQ ID NO: 33 - protein, 117 aa, synthetic construct
QVQLQQWGAGLLKPSETLSLTCAVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 34 - protein, 107 aa, synthetic construct
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIK

SEQ ID NO: 35 - protein, 130 aa, synthetic construct
QVQLQESGPGLVKPSQTLSLTCTVSDGSISSGGYYWSWIRQHPGKGLEWIGNIYYSGSTYYNPSLKSRVTISVDTSKNQFSLKLNSVTAADTAVYYCATEKALATVTPSGYENYYTVDVWGQGTTVTVSS

SEQ ID NO: 36 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQHLNSYPLTFGGGTKVEIK

SEQ ID NO: 37 - protein, 130 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCAREKALASVIPSGYENYYVVDVWGQGTTVTVSS

SEQ ID NO: 38 - protein, 107 aa, synthetic construct
DIQLTQSPSFLSASVGGRVTITCWASQGIAGYLAWYQQKPGKAPKLLIYAASTLQSGVPSRFSGSGSGTEFTLTISSLQPEDFATYYCQHLNSYPLTFGGGTKVEIK

SEQ ID NO: 39 - protein, 116 aa, synthetic construct
QVQLVESGGVLVKPGGSLRLSCAASGFTLSDYYMSWIRQAPGMGLEWVSYISSSGSTIYYTDSVKGRFTISRDSAKNSLYLQMNSLRAEDTAVYYCARDGVGFDYWGQGTLVTVSS

SEQ ID NO: 40 - protein, 109 aa, synthetic construct
EIVLTQSPGTLSLFPGERATLSCRASQSVSSSYLAWYQQKPGQSPRLLIYAASSRATGIPDRFSGSGSGTDFTLTISRLEPEDFAVYYCQQYGSSPLYTFGQGTKLEIK

SEQ ID NO: 41 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 107 aa, synthetic construct [sequence omitted]

SEQ ID NO: 43 - protein, 117 aa, synthetic construct
QVQLQQWGAGLLKPSETLSLTCTVYGGSFSGYYWNWIRQPPGRGLEWIGEINHSGSTSYNPSLKSRITISIDTSKNQFSLKLSSVTAADTAVYYCARVLYNWNVDSWGQGTLVTVSS

SEQ ID NO: 44 - protein, 214 aa, synthetic construct
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 117 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 46 - protein, 117 aa, synthetic construct
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSS

SEQ ID NO: 47 - protein, 117 aa, synthetic construct
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSS

SEQ ID NO: 48 - protein, 448 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 448 aa, synthetic construct
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 448 aa, synthetic construct
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 447 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 331 aa, synthetic construct; identical to SEQ ID NO: 41

SEQ ID NO: 53 - DNA, 858 bp, synthetic construct
  source 1..858: mol_type = other DNA
gccaccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataataa

SEQ ID NO: 54 - DNA, 846 bp, synthetic construct
  source 1..846: mol_type = other DNA
atggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaa

SEQ ID NO: 55 - protein, 282 aa, synthetic construct
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

(19) United States
(12) Patent Application Publication
(10) Pub. No.: US 2023/0310636 A1
(43) Pub. Date: Oct. 5, 2023

(54) COMBINATION THERAPIES FOR TREATMENT OF CANCER WITH THERAPEUTIC BINDING MOLECULES

(71) Applicant: MedImmune Limited, Cambridge (GB)

(72) Inventors: Krista Lynne KINNEER, Gaithersburg, MD (US); Elisabetta LEO, Cambridge (GB); Anne Frances TOSTO, Gaithersburg, MD (US); Kimberly COOK, Gaithersburg, MD (US); Jon CHESEBROUGH, Gaithersburg, MD (US)

(21) Appl. No.: 18/169,497

(22) Filed: Feb. 15, 2023

Related U.S. Application Data
(60) Provisional application No. 63/310,967, filed on Feb. 16, 2022.
(60) Provisional application No. 63/378,295, filed on Oct. 4, 2022.

(51) Int. Cl. A61K 47/68; A61K 31/496; A61P 35/00

(52) U.S. Cl. CPC A61K 47/6849; A61K 31/496; A61P 35/00

(57) ABSTRACT

Provided are methods of treating cancer in a subject, comprising administering to the subject: i) an antibody-drug conjugate (ADC), ii) a cytotoxic agent and iii) an additional agent, wherein the additional agent is a PARP1 inhibitor or an ATR inhibitor or a pharmaceutically acceptable salt thereof. The present disclosure further provides kits comprising i) an antibody-drug conjugate (ADC), ii) a cytotoxic agent and iii) an additional agent, wherein the additional agent is a PARP1 inhibitor or an ATR inhibitor or a pharmaceutically acceptable salt thereof.

### CROSS-REFERENCE TO RELATED APPLICATIONS

This application claims priority benefit of U.S. Provisional Patent Application No. 63/310,967, filed Feb. 16, 2022; and U.S. Provisional Patent Application No. 63/378,295, filed Oct. 4, 2022, which are incorporated by reference herein in their entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 54,905 Byte XML file named “B7H4-101-US—NP_SeqList.xml” created on Feb. 8, 2023.

### FIELD OF THE DISCLOSURE

The present disclosure provides a method of treating cancer in a human subject, comprising administering to the subject: i) an antibody-drug conjugate (ADC), ii) a cytotoxic agent and iii) an additional agent, wherein the additional agent is a PARP1 inhibitor or an ATR inhibitor or a pharmaceutically acceptable salt thereof. The present disclosure further provides kits comprising i) an antibody-drug conjugate (ADC), ii) a cytotoxic agent and iii) an additional agent, wherein the additional agent is a PARP1 inhibitor or an ATR inhibitor or a pharmaceutically acceptable salt thereof.

### BACKGROUND

Surgery, radiation therapy and chemotherapy, alone or in combination, are the most traditional and widely used treatment methods for cancer. While these treatment methods are effective in removing or killing cancer cells, they often result in undesirable side effects such as hair loss, anemia, severe nausea and death of healthy cells in patients treated. These limitations present an urgent need for innovative and less harmful treatment methods of cancer.

Antibody based cancer therapies rely on the recognition and binding of antibody-drug conjugates to specific proteins on cancer cells. Antibody drug conjugates (ADCs) can take advantage of the specificity of the antibody portion of the conjugate to deliver a highly toxic agent directly to the cells to be killed. Using antibody or ADC cancer therapies in combination with other small molecule based cancer therapies can improve treatment outcomes by attacking malignant cells and tumors in more than one way.

### SUMMARY OF THE DISCLOSURE

In some aspects, the present disclosure is directed to a method of treating cancer in a human subject in need thereof, comprising administering to the human subject:
A) an antibody-drug conjugate (ADC) comprising:
   i. an antibody or antigen binding fragment thereof which binds to a B7-H4 polypeptide, comprising:
      a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
      b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
      c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
      d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
      e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
   ii. a cleavable linker; and
   iii. a cytotoxic agent; and
B) an additional agent, wherein the additional agent is a PARP1 inhibitor or an ATR inhibitor or a pharmaceutically acceptable salt thereof. In some aspects, the additional agent is AZD5305. In some aspects, the additional agent is AZD6738.

In some aspects of the method, the cancer comprises a cancer cell which expresses B7-H4. In some aspects, the cancer further comprises a cancer cell that does not express B7-H4. In some aspects, the cancer is selected from ovarian cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutated HRD gene is selected from BRCA1, BRCA2, and ATM.

In some aspects of the method, the antibody or antigen binding fragment thereof comprises:
i. a variable heavy (VH) chain and a variable light (VL) chain comprising the amino acid sequence of SEQ ID NO: 45 and SEQ ID NO: 34, respectively, or a functional variant thereof;
ii. a variable heavy (VH) chain and a variable light (VL) chain comprising the amino acid sequence of SEQ ID NO: 33 and SEQ ID NO: 34, respectively, or a functional variant thereof;
iii. a variable heavy (VH) chain and a variable light (VL) chain comprising the amino acid sequence of SEQ ID NO: 43 and SEQ ID NO: 34, respectively, or a functional variant thereof;
iv. a variable heavy (VH) chain and a variable light (VL) chain comprising the amino acid sequence of SEQ ID NO: 46 and SEQ ID NO: 34, respectively, or a functional variant thereof;
v. a variable heavy (VH) chain and a variable light (VL) chain comprising the amino acid sequence of SEQ ID NO: 47 and SEQ ID NO: 34, respectively, or a functional variant thereof;
vi. a VH chain and a VL chain comprising the amino acid sequence of SEQ ID NO: 31, and SEQ ID NO: 32, respectively, or a functional variant thereof;
vii. a VH chain and a VL chain comprising the amino acid sequence of SEQ ID NO: 35 and SEQ ID NO: 36, respectively, or a functional variant thereof,
viii. a VH chain and a VL chain comprising the amino acid sequence of SEQ ID NO: 37 and SEQ ID NO: 38, respectively, or a functional variant thereof, or
ix, a VH chain and a VL chain comprising the amino acid sequence of SEQ ID NO: 39 and SEQ ID NO: 40, respectively, or a functional variant thereof.

In some aspects, the antibody or antigen binding fragment thereof comprises:
i. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In some aspects, the antibody or antigen binding fragment thereof comprises:
i. a VH chain and a VL chain comprising the amino acid sequence of SEQ ID NO: 45 and SEQ ID NO: 34, respectively, or a functional variant thereof.

In some aspects of the method, the antibody or antigen binding fragment thereof binds an OVCAR4 cell line.

In some aspects of the method, the antibody or antigen binding fragment thereof comprises a heavy chain constant region comprising the amino acid sequence of SEQ ID NO: 41. In some aspects, the antibody or antigen binding fragment thereof comprises a heavy chain constant region comprising the amino acid sequence of SEQ ID NO: 52. In some aspects, the antibody or antigen binding fragment thereof comprises a light chain constant region comprising the amino acid sequence of SEQ ID NO: 42. In some aspects, the antibody or antigen binding fragment thereof comprises a heavy chain comprising the amino acid sequence of SEQ ID NO: 51; and a light chain comprising the amino acid sequence of SEQ ID NO: 44. In some aspects, the antibody or antigen binding fragment thereof comprises a heavy chain comprising the amino acid sequence of SEQ ID NO: 48; and a light chain comprising the amino acid sequence of SEQ ID NO: 44.

In some aspects, the antibody or antigen binding fragment thereof is a monoclonal antibody. In some aspects, the antibody or antigen binding fragment thereof is a humanized monoclonal antibody.

In some aspects, the cleavable linker is an mp-PEG8-val-ala linker.

In some aspects, the cytotoxic agent is a topoisomerase inhibitor. In some aspects, the topoisomerase inhibitor is a compound of Formula A*

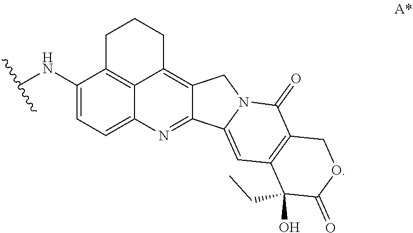 [SMILES: *Nc1ccc2nc3c(c4c2c1CCC4)Cn1c-3cc2c(c1=O)COC(=O)[C@]2(O)CC]

In some aspects of the method, the ii) linker and iii) cytotoxic agent are together selected from the following compounds:

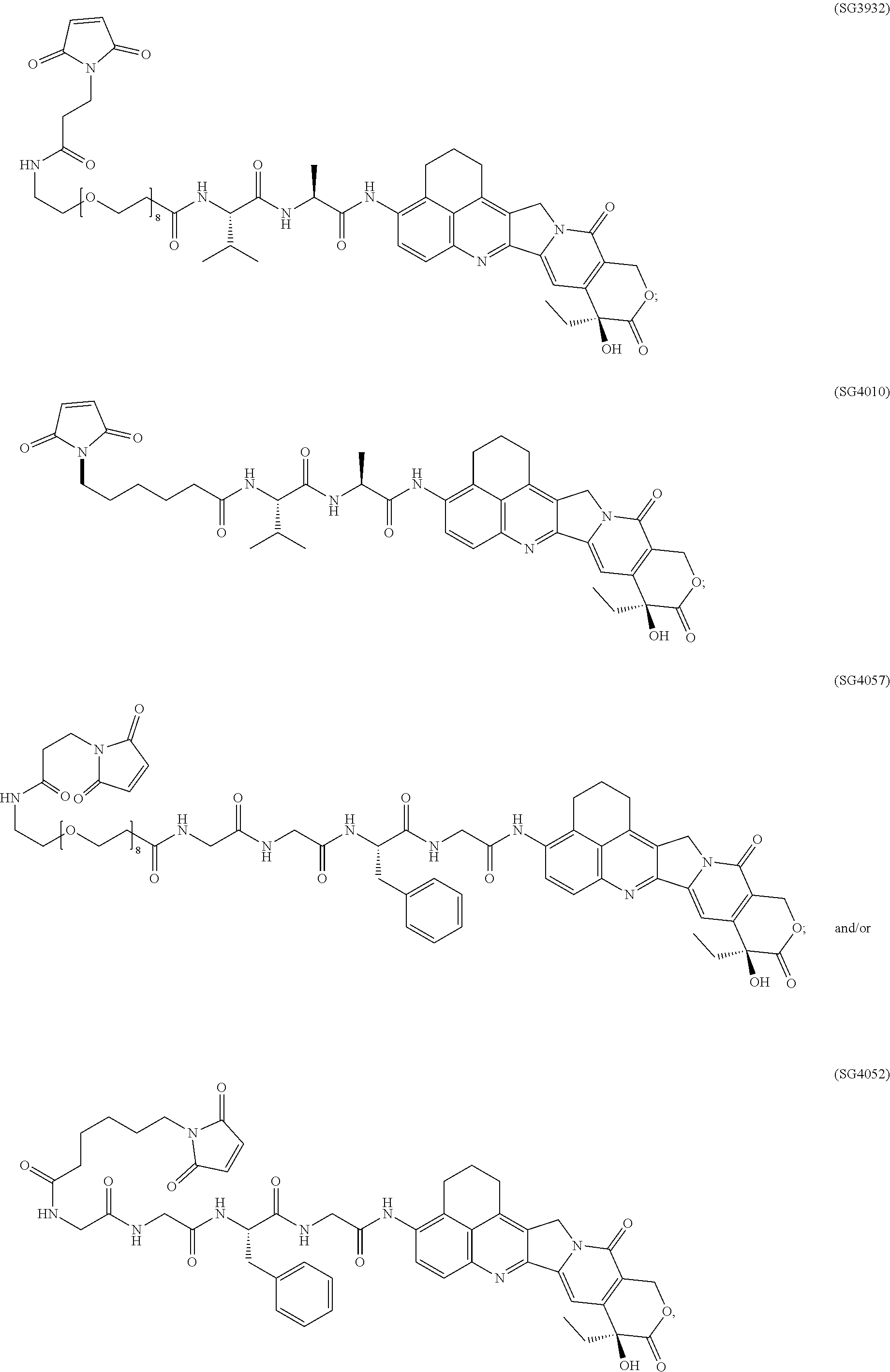

In some aspects of the method, the ii) linker and iii) cytotoxic agent are together the compound SG3932.

In some aspects of the method, the ADC has a drug to antibody ratio (DAR) of between about 1 and about 8. In some aspects, the ADC has a DAR of about 8.

In some aspects, the present disclosure is directed to a kit comprising:
A) an antibody-drug conjugate (ADC) comprising:
   i. an antibody or antigen binding fragment thereof which binds to a B7-H4 polypeptide comprising:
   a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
   b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof, c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof,
   d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof, or
   e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof,
   ii. a cleavable linker; and
   iii. a cytotoxic agent; and
   B) an additional agent, wherein the additional agent is a PARP1 inhibitor or an ATR inhibitor or a pharmaceutically acceptable salt thereof. In some aspects, the additional agent is AZD5305. In some aspects, the additional agent is AZD6738.

In some aspects of the kit, the antibody or antigen binding fragment thereof comprises:
i. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In some aspects of the kit, the antibody or antigen binding fragment thereof comprises:
i. a VH chain and a VL chain comprising the amino acid sequence of SEQ ID NO: 45 and SEQ ID NO: 34, respectively, or a functional variant thereof.

In some aspects of the kit, the antibody or antigen binding fragment thereof comprises a heavy chain constant region comprising the amino acid sequence of SEQ ID NO: 41. In some aspects, the antibody or antigen binding fragment thereof comprises a heavy chain constant region comprising the amino acid sequence of SEQ ID NO: 52. In some aspects, the antibody or antigen binding fragment thereof comprises a light chain constant region comprising the amino acid sequence of SEQ ID NO: 42. In some aspects, the antibody or antigen binding fragment thereof comprises a heavy chain comprising the amino acid sequence of SEQ ID NO: 51; and a light chain comprising the amino acid sequence of SEQ ID NO: 44. In some aspects, the antibody or antigen binding fragment thereof comprises a heavy chain comprising the amino acid sequence of SEQ ID NO: 48; and a light chain comprising the amino acid sequence of SEQ ID NO: 44.

In some aspects of the kit, the cleavable linker is an mp-PEG8-val-ala linker.

In some aspects of the kit, the cytotoxic agent is a topoisomerase inhibitor. In some aspects, the topoisomerase inhibitor is a compound of Formula A*

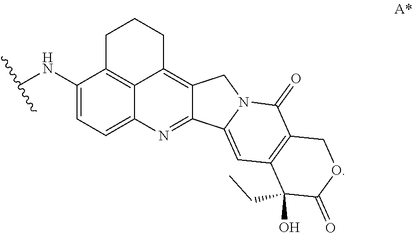 [SMILES: *Nc1ccc2nc3c(c4c2c1CCC4)Cn1c-3cc2c(c1=O)COC(=O)[C@]2(O)CC]

In some aspects of the kit, the ii) linker and iii) cytotoxic agent are together selected from the following compounds:

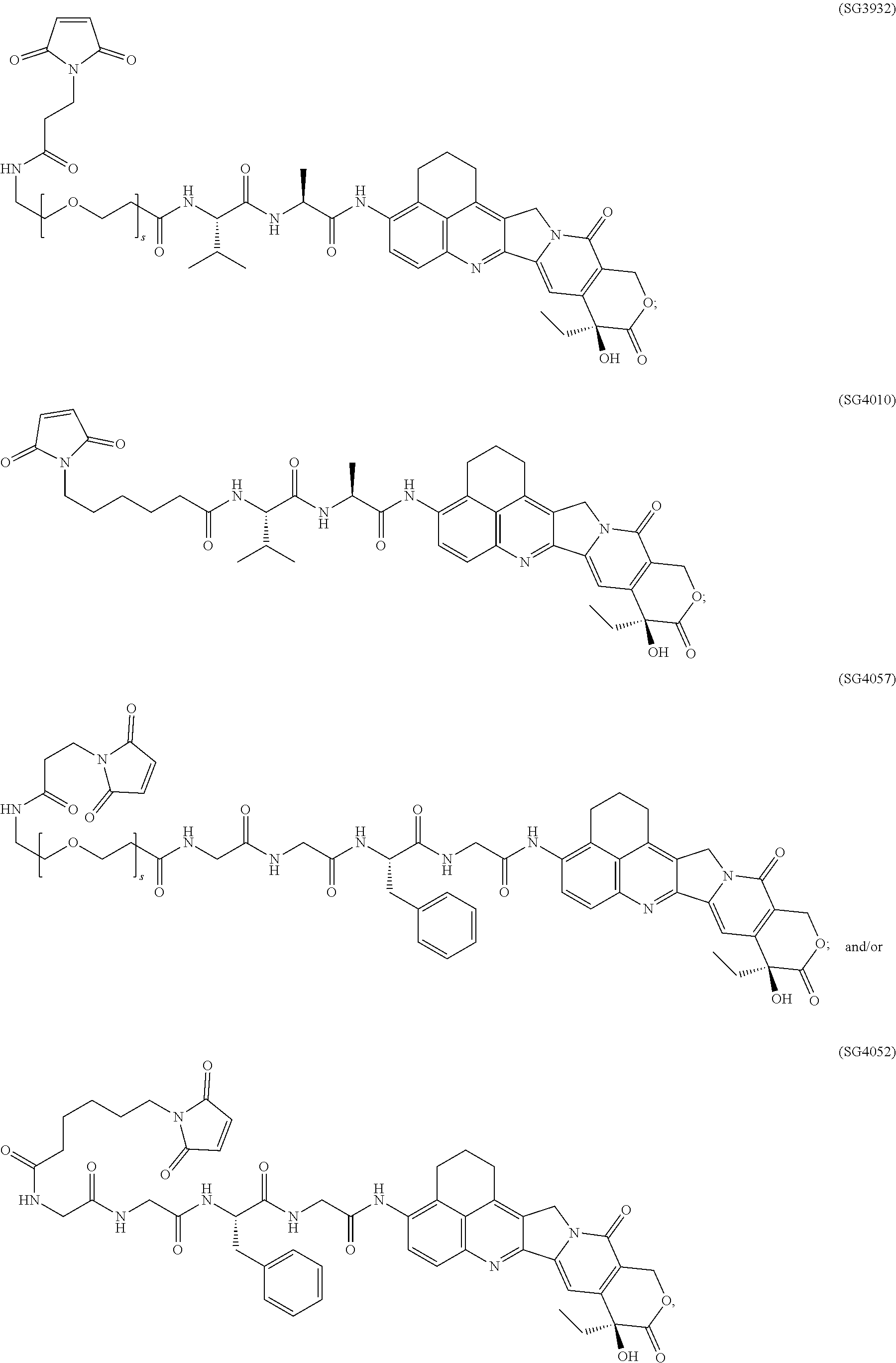

In some aspects of the kit, the ii) linker and iii) cytotoxic agent are together the compound SG3932.

In some aspects of the kit, the ADC has a drug to antibody ratio (DAR) of between about 1 and about 8. In some aspects, the ADC has a DAR of about 8.

In some aspects, the present disclosure is directed to a method of treating cancer in a human subject in need thereof, comprising administering to the human subject:
A) an antibody-drug conjugate (ADC) comprising:
   i. an antibody or antigen binding fragment thereof which binds to a B7-H4 polypeptide comprising:
      a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
      b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
      c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
      d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
      e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; and
   ii. a cleavable linker and cytotoxic agent conjugated to the antibody or antigen binding fragment thereof having the formula:

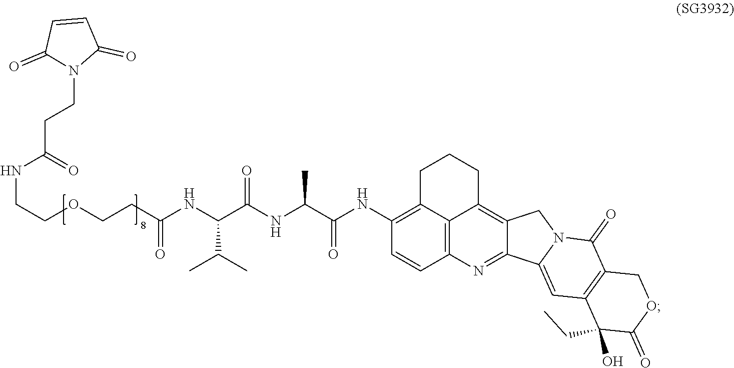

and
B) AZD5305 or a pharmaceutically acceptable salt thereof.

In some aspects, the present disclosure is directed to a method of treating cancer in a human subject in need thereof, comprising administering to the human subject:
A) an antibody-drug conjugate (ADC) comprising:
   i. an antibody or antigen binding fragment thereof which binds to a B7-H4 polypeptide comprising:
      f) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
      g) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
      h) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
      i) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
      j) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; and
   ii. a cleavable linker and cytotoxic agent conjugated to the antibody or antigen binding fragment thereof having the formula:

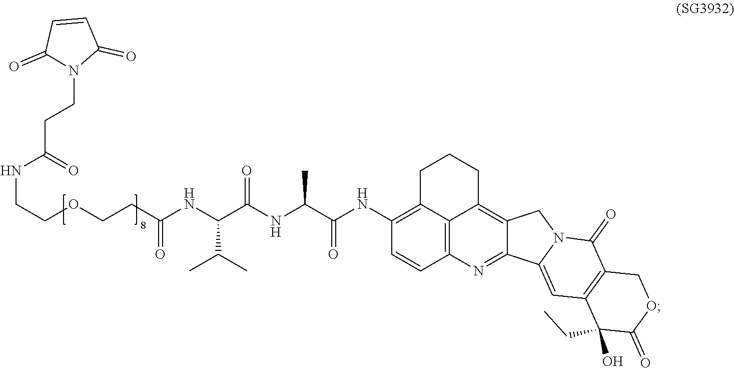

B) AZD6738 or a pharmaceutically acceptable salt thereof.

In some aspects of the method, the cancer comprises a cancer cell which expresses B7-H4. In some aspects, the cancer further comprises a cancer cell that does not express B7-H4. In some aspects, the cancer is selected from ovarian cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutated HRD gene is selected from BRCA1, BRCA2, and ATM.

### BRIEF DESCRIPTION OF THE DRAWINGS

The following drawings form part of the present specification and are included to further demonstrate exemplary aspects of certain some aspects of the present disclosure.

FIG. 1 shows results of the cytotoxic activity assay using E02-GL-SG3932 and AZD5305 treatments in DLD-1-BRCA wild-type cells engineered to express B7-H4.

FIG. 2 shows the Bliss synergy score matrix of DLD-1-BRCA wild-type cells treated with E02-GL-SG3932 and AZD5305 combination therapy.

FIG. 3 shows results of the cytotoxic activity assay using E02-GL-SG3932 and AZD5305 treatments in DLD-1-XMAN-BRCA2-/-cells engineered to express B7-H4.

FIG. 4 shows the Bliss synergy score matrix of DLD-1-XMAN-BRCA2-/-cells treated with E02-GL-SG3932 and AZD5305 combination therapy.

FIG. 5 shows results of the cytotoxic activity assay using E02-GL-SG3932 and AZD5305 treatments in MX-1 cells.

FIG. 6 shows the Bliss synergy score matrix of MX-1 cells treated with E02-GL-SG3932 and AZD5305 combination therapy.

FIG. 7 displays the in vivo anti-tumor efficacy of individual E02-GL-SG3932 and AZD5305 treatments in mouse model.

FIG. 8 shows the results of the in vivo anti-tumor efficacy experiments of combination therapies of E02-GL-SG3932 and AZD5305 in mouse model.

FIG. 9 displays the mean tumor volumes of the in vivo anti-tumor efficacy experiments of individual and combination therapies of E02-GL-SG3932 and AZD5305 in mouse model.

FIG. 10 displays the mean tumor volumes of the in vivo anti-tumor efficacy experiments of individual and combination therapies of E02-GL-SG3932 and AZD6738 in mouse model.

FIGS. 11A-11C shows results of B7-H4 expression in human tumors as described in Example 6. FIG. 11A: IHC was used to assess the expression of B7-H4 across multiple tumor types. The prevalence of B7-H4 expression in an indication is shown by the proportion of cells expressing B7-H4 positivity at any intensity. FIG. 11B: Representative images of B7-H4 IHC staining in endometrial cancer, cholangiocarcinoma, ER+ breast cancer or TNBC, and ovarian cancer. FIG. 11C: Quantitative image analysis showing the distribution of mean optical density (OD) of B7-H4 expression in each tumor cell membrane in primary tumor surgical resection, formalin-fixed, paraffin-embedded (FFPE) samples of TNBC (n=196). Each truncated violin plot represents an individual donor sample. Lines represent medians.

FIGS. 12A-12F shows results of E02-GL-SG3932 antitumor efficacy in combination with the PARP1-selective inhibitor AZD5305 in TNBC PDX models as described in Example 6. Tumors were established as described in Example 6, and treatments were administered at the doses indicated in each panel. ADCs were delivered as a single bolus IV injection, and AZD5305 was delivered by oral gavage once daily for 28 days. FIGS. 12A-12B: Antitumor efficacy resulting from (A) 1.25 mg/kg or (B) 3.5 mg/kg ADC treatment alone or in combination with AZD5305 in the high B7-H4-expressing BRCA WT HBCx-39 model. FIGS. 12C-12D: Activity resulting from treatment of (C) high B7-H4-expressing BRCA1-mutant HBCx-24 and (D) BRCA1-hypomorphic HBCx-11 models. FIGS. 12E-12F: Efficacy resulting from treatment of (E) HBCx-8, a BRCA1-mutant model, and (F) HBCx-2, a BRCA WT model with low B7-H4 expression. Data indicate tumor volume mean±standard error of the mean (n=4 or 5 animals per group).

### DETAILED DESCRIPTION

Disclosed herein are methods, combinations and kits comprising administration of an antibody-drug conjugate (ADC) together with a second agent such as a small molecule drug. The methods combinations and kits can be used in the treatment of cancer in a subject as described herein.

In some aspects, the disclosure provides a method of treating cancer in a human subject in need thereof, comprising administering to the human subject: A) an antibody-drug conjugate (ADC) comprising: i. an antibody or antigen binding fragment thereof which binds to a B7-H4 polypeptide, ii. a cleavable linker; and iii. a cytotoxic agent; and B) a PARP1 (Poly(ADP-Ribose) Polymerase 1) inhibitor. In some aspects, the disclosure provides a method of treating cancer in a human subject in need thereof, comprising administering to the human subject: A) an antibody-drug conjugate (ADC) comprising: i. an antibody or antigen binding fragment thereof which binds to a B7-H4 polypeptide, ii. a cleavable linker; and iii. a cytotoxic agent; and B) an ATR (FRAP-Related Protein 1; FRP1; MEC1; SCKL; SECKL1R) inhibitor.

In some aspects, the disclosure provides a method of treating cancer in a human subject in need thereof, comprising administering to the human subject: A) an ADC comprising: i. an antibody or antigen binding fragment thereof which binds to a B7-H4 polypeptide, ii. a cleavable linker; and iii. a cytotoxic agent; and B) AZD5305 or a pharmaceutically acceptable salt thereof. In some aspects, the disclosure provides a method of treating cancer in a human subject in need thereof, comprising administering to the human subject: A) an ADC comprising: i. an antibody or antigen binding fragment thereof which binds to a B7-H4 polypeptide, ii. a cleavable linker; and iii. a cytotoxic agent; and B) AZD6738 or a pharmaceutically acceptable salt thereof.

In some aspects, the disclosure provides a method of treating cancer in a human subject in need thereof, comprising administering to the human subject: A) an ADC comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof; c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof, d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD5305 or a pharmaceutically acceptable salt thereof.

In some aspects, the disclosure provides a method of treating cancer in a human subject in need thereof, comprising administering to the human subject: A) an ADC comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof; c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof, d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD6738 or a pharmaceutically acceptable salt thereof.

### Treatment of Cancers

In some aspects, disclosed herein is a method of treating a cancer that is associated with the expression of B7-H4. In some aspects, the cancer comprises a cancer cell which expresses B7-H4. In some aspects, the cancer is a tumor or other mass of malignant cells comprising a cancer cell which expresses B7-H4. In some aspects, the cancer further comprises a cancer cell that does not express B7-H4.

B7-H4 (also known as V-set domain-containing T-cell activation inhibitor 1, encoded by the VTCN1 gene) is a transmembrane polypeptide of the B7 family of co-stimulatory proteins. B7-H4 is understood to be expressed on the surface of antigen-presenting cells for interactions with ligands of immune cells (e.g., T-lymphocytes, with CD28 being a potential ligand). B7-H4 has been observed to be highly expressed on cells of various cancer types and is thought to be a tumour-associated antigen. Furthermore, B7-H4 expression is not limited to a particular cancer type, such that it represents a target antigen for treating a broad spectrum of cancer types.

In a preferable aspect, a cancer referred to herein is a cancer characterised by the expression (preferably overexpression) of a B7-H4 molecule. In other words, a cancer referred to herein may comprise a cancerous cell that expresses B7-H4. Said cancerous cell may be comprised within a tumor. In another aspect, the B7-H4 molecule is expressed in the cancer cell at a level similar to the level of expression in a non-cancer cell. In another aspect, the B7-H4 molecule is expressed in the cancer cell at a level lower than the level of expression in a non-cancer cell.

To “treat” refers to therapeutic measures that cure, slow down, alleviate symptoms of, and/or halt progression of a diagnosed pathologic condition or disorder. Thus, those in need of treatment include those already with the disorder. In some aspects, a subject is successfully “treated” for a disease or disorder (preferably cancer), according to the methods provided herein if the patient shows, e.g., total, partial, or transient alleviation or elimination of symptoms associated with the disease or disorder (preferably cancer).

In some aspects, a method of the disclosure may be used to prevent the onset of a cancer comprising a cancer cell which expresses B7-H4. To “prevent” refers to prophylactic or preventative measures that prevent and/or slow the development of a targeted pathologic condition or disorder. Thus, those in need of prevention include those prone to have or susceptible to the disorder. In some aspects, a disease or disorder (preferably cancer) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the disclosure.

The terms “subject”, “individual” and “patient” are used interchangeably herein to refer to a mammalian subject. In some aspects the “subject” is a human, domestic animals, farm animals, sports animals, and zoo animals, e.g., humans, non-human primates, dogs, cats, guinea pigs, rabbits, rats, mice, horses, cattle, etc. In some aspects, the subject is a cynomolgus monkey (Macaca fascicularis). In a preferable aspect, the subject is a human. In methods of the disclosure, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, In some aspects, a method of the disclosure may be used to confirm the presence of cancer in a subject. For example, the subject may previously have been diagnosed with cancer by alternative means. In some aspects, the subject has been previously administered a cancer therapy.

In some aspects, disclosed herein is a method of treating a cancer selected from ovarian cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is ovarian cancer. In some aspects of the method for treating breast cancer, breast cancer is a hormone receptor-positive (HR+) breast cancer, human epidermal growth factor receptor 2 positive (HER2+) breast cancer, or a triple negative breast cancer (TNBC). In some aspects, the breast cancer is TNBC.

In some aspects of the method, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutated HRD gene is selected from BRCA1, BRCA2, and ATM. In some aspects, the mutated HRD gene is BRCA1. In some aspects, the mutated HRD gene is BRCA2. In some aspects, the mutated HRD gene is ATM

### Antibodies and Antigen Binding Fragments

The antibody drug conjugates provided herein comprise an antibody or antigen binding fragment thereof that binds to a B7-H4 polypeptide. The RNA, DNA, and amino acid sequences of B7-H4 are known to those skilled in the art and can be found in many databases, for example, in the databases of the National Center for Biotechnology Information (NCBI) and UniProt. Examples of these sequences found at UniProt are at Q7Z7D3 (VTCN1_HUMAN) for human B7-H4; and Q7TSP5 (VTCN1_MOUSE) for mouse B7-H4. The nucleotide sequence encoding for human B7-H4 may be SEQ ID NO: 53, more preferably SEQ ID NO: 54. The polypeptide sequence of human B7-H4 is preferably SEQ ID NO: 55.

In some aspects, the antibody or antigen binding fragment thereof comprises a HCDR1, a HCDR2, a HCDR3, a LCDR1, LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof. An antibody or antigen binding fragment thereof comprising said sequences may be referred to as “ZYOEPQ-E02” or “EPQ-E02” herein.

In some aspects, the antibody or antigen binding fragment thereof comprises a HCDR1, a HCDR2, a HCDR3, a LCDR1, LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof. An antibody or antigen binding fragment thereof comprising said sequences may be referred to as “ZYOEOB-F05” or “EOB-F05” herein.

In some aspects, the antibody or antigen binding fragment thereof comprises a HCDR1, a HCDR2, a HCDR3, a LCDR1, LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof. An antibody or antigen binding fragment thereof comprising said sequences may be referred to as “ZY0E05-E07” or “E05-E07” herein.

In some aspects, the antibody or antigen binding fragment thereof comprises a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof. An antibody or antigen binding fragment thereof comprising said sequences may be referred to as “ZYOEPO-C07” or “EPO-C07” herein.

In a particular aspect, the antibody or antigen binding fragment thereof comprises a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In other words, the antibody or antigen binding fragment thereof may preferably comprise:
i. a HCDR1 comprising the amino acid sequence of SEQ ID NO: 7, or a functional variant thereof;
ii. a HCDR2 comprising the amino acid sequence of SEQ ID NO: 8, or a functional variant thereof;
iii. a HCDR3 comprising the amino acid sequence of SEQ ID NO: 9, or a functional variant thereof;
iv. a LCDR1 comprising the amino acid sequence of SEQ ID NO: 10, or a functional variant thereof;
v. a LCDR2 comprising the amino acid sequence of SEQ ID NO: 11, or a functional variant thereof; and
vi. a LCDR3 comprising the amino acid sequence of SEQ ID NO: 12, or a functional variant thereof.

In some aspects, the antibody or antigen binding fragment thereof comprises:
i. a HCDR1 comprising the amino acid sequence of SEQ ID NO: 1, or a functional variant thereof;
ii. a HCDR2 comprising the amino acid sequence of SEQ ID NO: 2, or a functional variant thereof;
iii. a HCDR3 comprising the amino acid sequence of SEQ ID NO: 3, or a functional variant thereof;
iv. a LCDR1 comprising the amino acid sequence of SEQ ID NO: 4, or a functional variant thereof;
v. a LCDR2 comprising the amino acid sequence of SEQ ID NO: 5, or a functional variant thereof; and
vi. a LCDR3 comprising the amino acid sequence of SEQ ID NO: 6, or a functional variant thereof.

In some aspects, the antibody or antigen binding fragment thereof comprises:
i. a HCDR1 comprising the amino acid sequence of SEQ ID NO: 13, or a functional variant thereof;
ii. a HCDR2 comprising the amino acid sequence of SEQ ID NO: 14, or a functional variant thereof;
iii. a HCDR3 comprising the amino acid sequence of SEQ ID NO: 15, or a functional variant thereof;
iv. a LCDR1 comprising the amino acid sequence of SEQ ID NO: 16, or a functional variant thereof;
v. a LCDR2 comprising the amino acid sequence of SEQ ID NO: 17, or a functional variant thereof; and
vi. a LCDR3 comprising the amino acid sequence of SEQ ID NO: 18, or a functional variant thereof.

In some aspects, the antibody or antigen binding fragment thereof comprises:
i. a HCDR1 comprising the amino acid sequence of SEQ ID NO: 19, or a functional variant thereof;
ii. a HCDR2 comprising the amino acid sequence of SEQ ID NO: 20, or a functional variant thereof;
iii. a HCDR3 comprising the amino acid sequence of SEQ ID NO: 21, or a functional variant thereof;
iv. a LCDR1 comprising the amino acid sequence of SEQ ID NO: 22, or a functional variant thereof;
v. a LCDR2 comprising the amino acid sequence of SEQ ID NO: 23, or a functional variant thereof; and
vi. a LCDR3 comprising the amino acid sequence of SEQ ID NO: 24, or a functional variant thereof.

In some aspects, the antibody or antigen binding fragment thereof comprises:
i. a HCDR1 comprising the amino acid sequence of SEQ ID NO: 25, or a functional variant thereof;
ii. a HCDR2 comprising the amino acid sequence of SEQ ID NO: 26, or a functional variant thereof;
iii. a HCDR3 comprising the amino acid sequence of SEQ ID NO: 27, or a functional variant thereof;
iv. a LCDR1 comprising the amino acid sequence of SEQ ID NO: 28, or a functional variant thereof;
v. a LCDR2 comprising the amino acid sequence of SEQ ID NO: 29, or a functional variant thereof; and
vi. a LCDR3 comprising the amino acid sequence of SEQ ID NO: 30, or a functional variant thereof.

Additionally or alternatively, an antibody or antigen binding fragment thereof described herein may be described by means of a variable heavy (VH) chain and a variable light (VL) chain thereof.

Suitable a variable heavy (VH) chain sequences (which the antibody or antigen binding fragment thereof may comprise) are outlined in an individualized manner below:
SEQ ID NO: 31, or a functional variant thereof,
SEQ ID NO: 33, or a functional variant thereof
SEQ ID NO: 43, or a functional variant thereof
SEQ ID NO: 45, or a functional variant thereof
SEQ ID NO: 46, or a functional variant thereof
SEQ ID NO: 47, or a functional variant thereof
SEQ ID NO: 35, or a functional variant thereof
SEQ ID NO: 37, or a functional variant thereof
SEQ ID NO: 39, or a functional variant thereof

Particularly suitable variable heavy (VH) chain sequences (which the antibody or antigen binding fragment thereof may comprise) are outlined in an individualized manner below:
SEQ ID NO: 45, or a functional variant thereof
SEQ ID NO: 33, or a functional variant thereof
SEQ ID NO: 43, or a functional variant thereof
SEQ ID NO: 46, or a functional variant thereof
SEQ ID NO: 47, or a functional variant thereof

Suitable variable light (VL) chain sequences (which the antibody or antigen binding fragment thereof may comprise) are outlined in an individualised manner below:
SEQ ID NO: 32, or a functional variant thereof
SEQ ID NO: 34, or a functional variant thereof
SEQ ID NO: 36, or a functional variant thereof
SEQ ID NO: 38, or a functional variant thereof
SEQ ID NO: 40, or a functional variant thereof

A preferred variable light (VL) chain sequence (which the antibody or antigen binding fragment thereof may comprise) may comprise an amino acid sequence of SEQ ID NO: 34 (or a functional variant thereof).

For example, In some aspects, the antibody or antigen binding fragment thereof comprises:
i. a variable heavy chain comprising an amino acid sequence having at least 70%, 75%, 80%, 90%, 95% or 100% sequence identity to the amino acid sequence of SEQ ID NO: 31, 33, 35, 37, or 39, or a functional variant thereof, and
ii. a variable light chain comprising an amino acid sequence having at least 70%, 75%, 80%, 90%, 95% or 100% sequence identity to the amino acid sequence of SEQ ID NO: 32, 34, 36, 38, or 40, or a functional variant thereof.

For example, In some aspects, the antibody or antigen binding fragment thereof comprises:
i. a variable heavy chain comprising an amino acid sequence having at least 70%, 75%, 80%, 90%, 95% or 100% sequence identity to the amino acid sequence of SEQ ID NO: 31, 33, 35, 37, 39, 43, 45, 46, or 47, or a functional variant thereof; and
ii. a variable light chain comprising an amino acid sequence having at least 70%, 75%, 80%, 90%, 95% or 100% sequence identity to the amino acid sequence of SEQ ID NO: 32, 34, 36, 38, or 40, or a functional variant thereof.

Suitably, the antibody or antigen binding fragment thereof may comprise:
i. a variable heavy chain comprising an amino acid sequence having at least 70%, 75%, 80%, 90%, 95% or 100% sequence identity to the amino acid sequence SEQ ID NO: 33, or a functional variant thereof; and
ii. a variable light chain comprising an amino acid sequence having at least 70%, 75%, 80%, 90%, 95% or 100% sequence identity to the amino acid sequence of SEQ ID NO: 34, or a functional variant thereof.

More suitably, the antibody or antigen binding fragment thereof may comprise:
i. a variable heavy chain comprising an amino acid sequence having at least 70%, 75%, 80%, 90%, 95% or 100% sequence identity to the amino acid sequence SEQ ID NO: 45, or a functional variant thereof; and
ii. a variable light chain comprising an amino acid sequence having at least 70%, 75%, 80%, 90%, 95% or 100% sequence identity to the amino acid sequence of SEQ ID NO: 34, or a functional variant thereof.

In some aspects, the antibody or antigen binding fragment thereof comprises:
a variable heavy (VH) chain and a variable light (VL) chain comprising the amino acid sequence of SEQ ID NO: 31, and SEQ ID NO: 32, respectively, or a functional variant thereof;
a VH chain and a VL chain comprising the amino acid sequence of SEQ ID NO: 33 and SEQ ID NO: 34, respectively, or a functional variant thereof;
a VH chain and a VL chain comprising the amino acid sequence of SEQ ID NO: 43 and SEQ ID NO: 34, respectively, or a functional variant thereof;
a VH chain and a VL chain comprising the amino acid sequence of SEQ ID NO: 45 and SEQ ID NO: 34, respectively, or a functional variant thereof;
a VH chain and a VL chain comprising the amino acid sequence of SEQ ID NO: 46 and SEQ ID NO: 34, respectively, or a functional variant thereof,
a VH chain and a VL chain comprising the amino acid sequence of SEQ ID NO: 47 and SEQ ID NO: 34, respectively, or a functional variant thereof;
a VH chain and a VL chain comprising the amino acid sequence of SEQ ID NO: 35 and SEQ ID NO: 36, respectively, or a functional variant thereof;
a VH chain and a VL chain comprising the amino acid sequence of SEQ ID NO: 37 and SEQ ID NO: 38, respectively, or a functional variant thereof; or
a VH chain and a VL chain comprising the amino acid sequence of SEQ ID NO: 39 and SEQ ID NO: 40, respectively, or a functional variant thereof.

In a preferable aspect the antibody or antigen binding fragment thereof comprises: a variable heavy (VH) chain comprising the amino acid sequence of SEQ ID NO: 45, 33, 43, 46 or 47 (or a functional variant thereof); and a variable light (VL) chain comprising the amino acid sequence of SEQ ID NO: 34 (or a functional variant thereof). For example, the VH of SEQ ID NOs: 33, 45, 46 and/47 may correspond to “germlined” versions of the VH of SEQ ID NO: 33 (e.g., all having same CDR sequences, but with framework variations). Advantageously, each variant retains equivalent binding properties.

In some aspects, the antibody or antigen binding fragment thereof comprises: a variable heavy chain comprising the amino acid sequence of SEQ ID NO: 31, or a functional variant thereof, and a variable light chain comprising the amino acid sequence of SEQ ID NO: 32, or a functional variant thereof. An antibody or antigen binding fragment thereof comprising said sequences may be referred to as “ZYOEPD-E02” or “EPD-E02”.

In some aspects, the antibody or antigen binding fragment thereof comprises: a variable heavy chain comprising the amino acid sequence of SEQ ID NO: 35, or a functional variant thereof, and a variable light chain comprising the amino acid sequence of SEQ ID NO: 36, or a functional variant thereof. An antibody or antigen binding fragment thereof comprising said sequences may be referred to as “ZYOEOB-F05” or “EOB-F05”.

In some aspects, the antibody or antigen binding fragment thereof comprises: a variable heavy chain comprising the amino acid sequence of SEQ ID NO: 37, or a functional variant thereof, and a variable light chain comprising the amino acid sequence of SEQ ID NO: 38, or a functional variant thereof. An antibody or antigen binding fragment thereof comprising said sequences may be referred to as “ZY0E05-E07” or “E05-E07”.

In some aspects, the antibody or antigen binding fragment thereof comprises: a variable heavy chain comprising the amino acid sequence of SEQ ID NO: 39, or a functional variant thereof, and a variable light chain comprising the amino acid sequence of SEQ ID NO: 40, or a functional variant thereof. An antibody or antigen binding fragment thereof comprising said sequences may be referred to as “ZYOEPO-C07” or “EPO-C07”.

In some aspects, the antibody or antigen binding fragment thereof comprises: a variable heavy chain comprising the amino acid sequence of SEQ ID NO: 33, or a functional variant thereof, and a variable light chain comprising the amino acid sequence of SEQ ID NO: 34, or a functional variant thereof.

In some aspects, the antibody or antigen binding fragment thereof comprises: a variable heavy chain comprising the amino acid sequence of SEQ ID NO: 43, or a functional variant thereof, and a variable light chain comprising the amino acid sequence of SEQ ID NO: 34, or a functional variant thereof.

In some aspects, the antibody or antigen binding fragment thereof comprises: a variable heavy chain comprising the amino acid sequence of SEQ ID NO: 46, or a functional variant thereof, and a variable light chain comprising the amino acid sequence of SEQ ID NO: 34, or a functional variant thereof.

In some aspects, the antibody or antigen binding fragment thereof comprises: a variable heavy chain comprising the amino acid sequence of SEQ ID NO: 47, or a functional variant thereof; and a variable light chain comprising the amino acid sequence of SEQ ID NO: 34, or a functional variant thereof.

In a preferable aspect, the antibody or antigen binding fragment thereof comprises: a variable heavy chain comprising the amino acid sequence of SEQ ID NO: 45, or a functional variant thereof; and a variable light chain comprising the amino acid sequence of SEQ ID NO: 34, or a functional variant thereof. An antibody or antigen binding fragment thereof comprising said sequences may be referred to as “EQD-E02 GL”.

In some aspects, the antibody or antigen binding fragment thereof comprises a variable heavy chain comprising an amino acid sequence having at least 70%, 75%, 80%, 90%, 95% or 100% sequence identity to a reference amino acid sequence of SEQ ID NO: 43. In some aspects, the antibody or antigen binding fragment thereof comprises a variable heavy chain comprising an amino acid sequence of SEQ ID NO: 43. For example, the antibody or antigen binding fragment thereof may comprise a variable heavy chain comprising an amino acid sequence of SEQ ID NO: 43, and a variable light chain comprising an amino acid sequence of SEQ ID NO: 34.

Additionally or alternatively, an antibody or antigen binding fragment thereof described herein may be described by means of a heavy chain and/or light chain thereof.

In some aspects, the antibody or antigen binding fragment thereof comprises a light chain (e.g., comprising a VL and constant light chain) comprising an amino acid sequence having at least 70%, 75%, 80%, 90%, 95% or 100% sequence identity to the amino acid sequence of SEQ ID NO: 44. In a preferable aspect, the antibody or antigen binding fragment thereof comprises a light chain (e.g., comprising a VL and constant light chain) comprising the amino acid sequence of SEQ ID NO: 44.

In some aspects, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 48. For example, the antibody or antigen binding fragment thereof may comprise a heavy chain (e.g. comprising a VH and constant heavy chain) comprising the amino acid sequence of SEQ ID NO: 48. Such heavy chain may be referred to as “E02-GL-Maia-heavy chain”.

In some aspects, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 49. For example, the antibody or antigen binding fragment thereof may comprise a heavy chain (e.g. comprising a VH and constant heavy chain) comprising the amino acid sequence of SEQ ID NO: 49. Such heavy chain may be referred to as “E02-GLY-Maia-heavy chain”.

In some aspects, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 50. For example, the antibody or antigen binding fragment thereof may comprise a heavy chain (e.g. comprising a VH and constant heavy chain) comprising the amino acid sequence of SEQ ID NO: 50. Such heavy chain may be referred to as “E02-GLQ-Maia-heavy chain”.

In a preferred aspect, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 51. In a more preferable aspect, the antibody or antigen binding fragment thereof comprises a heavy chain (e.g. comprising a VH and constant heavy chain) comprising the amino acid sequence of SEQ ID NO: 51. Such heavy chain may be referred to as “E02-GL-WT-heavy chain”.

In some aspects, the antibody or antigen binding fragment thereof comprises a light chain constant region comprising an amino acid sequence having at least 70%, 75%, 80%, 90%, 95% or 100% sequence identity to a reference amino acid sequence of SEQ ID NO: 42. In a preferable aspect, the antibody or antigen binding fragment thereof comprises light chain constant region comprising an amino acid sequence of SEQ ID NO: 42.

In some aspects, the antibody or antigen binding fragment thereof comprises a heavy chain constant region comprising the amino acid sequence of SEQ ID NO: 41. More preferably, the antibody or antigen binding fragment thereof comprises a heavy chain constant region comprising the amino acid sequence of SEQ ID NO: 52.

In a preferable aspect, the antibody or antigen binding fragment thereof comprises a light chain (e.g., comprising a VL and constant light chain) comprising the amino acid sequence of SEQ ID NO: 44 and a heavy chain (e.g., comprising a VH and constant heavy chain) comprising the amino acid sequence of SEQ ID NO: 51.

Advantageously, it has been demonstrated that an antibody or antigen binding fragment of the claims may target a broader spectrum of B7-H4 expressing cells when compared with existing (commercially) available antibodies reported to target B7-H4. Thus, not only have the antibodies (or antigen binding fragments thereof) disclosed herein been demonstrated to have affinity and specificity for a clinically relevant target, but they have also been demonstrated to have a unique advantage (e.g., unexpected technical effect) associated therewith.

Preferably, antibody or antigen binding fragment thereof described herein is capable of binding to B7-H4 as an integral component of a cancer cell (for example, B7-H4 as an integral component of a cell membrane of a cancer cell).

An antibody or antigen binding fragment thereof described herein may bind to an OVCAR4 cell line and/or a CHO cell line (e.g., which may lack an exogenous nucleic acid encoding B7-H4). For example, the antibody or antigen binding fragment thereof binds to a B7-H4 (e.g., a B7-H4 epitope) of an OVCAR4 cell line and/or a CHO cell line (e.g., which may lack an exogenous nucleic acid encoding B7-H4). Suitably, the antibody or antigen binding fragment thereof described herein may bind to an OVCAR4 cell line and a CHO cell line (e.g., which may lack an exogenous nucleic acid encoding B7-H4).

The term “epitope” refers to a target protein region (e.g., polypeptide) capable of binding to (e.g., being bound by) an antibody or antigen binding fragment of the disclosure.

In some aspects, the antibody or antigen binding fragment thereof binds to an OVCAR4 cell line and/or CHO cell line (e.g. which may lack an exogenous nucleic acid encoding B7-H4) with higher affinity when compared to one or more antibody selected from E Biosciences 14-5949 anti-Human B7H4 mouse IgG, US biological B0000-35B anti Human B7H4 mouse IgG, R and D systems AF2514 anti-Mouse B7H4 goat IgG1, Sigma SAB2500141 anti B7H4 Goat IgG1, Isotype 1 CAT004 SP06-003, Isotype 2 R and D Normal goat IgG control (AB-108C), AdD serotec MCA2632, Epitomics 2516-1, eBiosciences, 145972-82, eBioscience 145970-85, or a combination thereof. For example, the antibody or antigen binding fragment thereof may bind to an OVCAR4 cell line and/or CHO cell line (e.g., which may lack an exogenous nucleic acid encoding B7-H4) with higher affinity when compared to one or more antibody selected from E Biosciences 14-5949 anti-Human B7H4 mouse IgG, US biological B0000-35B anti Human B7H4 mouse IgG, R and D systems AF2514 anti-Mouse B7H4 goat IgG1, and Sigma SAB2500141 anti B7H4 Goat IgG1, or a combination thereof.

In a preferable aspect, the antibody or antigen binding fragment thereof binds to an OVCAR4 cell line with higher affinity when compared to E Biosciences 14-5949 anti-Human B7H4 mouse IgG.

Reference to “E Biosciences 14-5949 anti-Human B7H4 mouse IgG” may be used interchangeably with the term “B7-H4 Monoclonal Antibody (H74), eBioscience” herein. Said antibody is available from ThermoFisher Scientific (Catalog #14-5949-82).

In another preferable aspect, the antibody or antigen binding fragment thereof binds to an OVCAR4 cell line with higher affinity when compared to US biological B0000-35B anti Human B7H4 mouse IgG.

Said affinity (e.g., binding affinity) can be measured by any suitable method of measuring binding affinity described herein.

The OVCAR4 cell line is a human ovary carcinoma cell line. The OVCAR4 cell line is obtainable from the National Cancer Institute for the transfer of cell lines from the Division of Cancer Treatment and Diagnosis Tumor Repository. The Chinese hamster ovary (CHO) cell line is an epithelial cell line derived from the ovary of the Chinese hamster, and is widely obtainable.

In some aspects, the antibody or antigen binding fragment thereof is a monoclonal antibody.

A “monoclonal antibody” (mAb) refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals.

In a preferable aspect, the antibody or antigen binding fragment thereof (e.g., mAb) of the disclosure is a humanized antibody or antigen binding fragment thereof. Suitably, said humanized the antibody or antigen binding fragment thereof is an IgG.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FW) connected by three complementarity-determining regions (CDRs), also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The “Kabat numbering system” is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The table below lists the positions of the amino acids comprising the variable regions of the antibodies in each system.

| Region | Kabat | AbM | Chothia |
|---|---|---|---|
| LCDR1 | L24-L34 | L24-L34 | L24-L34 |
| LCDR2 | L50-L56 | L50-L56 | L50-L56 |
| LCDR3 | L89-L97 | L89-L97 | L89-L97 |
| HCDR1¹ | H31-H35B | H26-H35B | H26-H32 . . . 34 |
| HCDR1² | H31-H35 | H26-H35 | H26-H32 |
| HCDR2 | H50-H65 | H50-H58 | H52-H56 |
| HCDR3 | H95-H102 | H95-H102 | H95-H102 |

¹Kabat Numbering
²Chothia Numbering

ImMunoGeneTics (IMGT) also provides a numbering system for the immunoglobulin variable regions, including the CDRs. See, e.g., Lefranc, M. P. et al., Dev. Comp. Immunol. 27: 55-77(2003). The IMGT numbering system is based on an alignment of more than 5,000 sequences, structural data, and characterization of hypervariable loops and allows for easy comparison of the variable and CDR regions for all species. According to the IMGT numbering schema, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is a positions 50-65, and VH-CDR3 is at positions 95-102. VL-CDR1, VL-CDR2 and VL-CDR3 also correspond to classical Kabat numbering locations, namely positions 24-34, 50-56 and 89-97, respectively.

In some aspects, an antibody of the disclosure a human antibody.

The term “human antibody” means an antibody produced in a human or an antibody having an amino acid sequence corresponding to an antibody produced in a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

In some aspects, an antibody of the disclosure a chimeric antibody.

The term “chimeric antibodies” refers to antibodies in which the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006); Robbie et al., (2013) Antimicrob. Agents Chemother. 57, 6147-6153). See also U.S. Pat. No. 7,083,784, which is hereby incorporated by reference in its entirety.

Suitably, the antibody or antigen binding fragment of the disclosure binds to B7-H4 molecule with sufficient affinity such that the antibody is useful as a therapeutic agent or a diagnostic reagent in targeting B7-H4.

In some aspects, the antibody or antigen binding fragment thereof binds to a B7-H4 (preferably a human B7-H4) with a dissociation constant (KD) of ≤1 μM, ≤100 nM, ≤10 nM, ≤1 nM, ≤0.1 nM, ≤10 pM, ≤1 pM, or 0.1 pM. In some aspects, the antibody or antigen binding fragment thereof binds to a B7-H4 (preferably a human B7-H4) with a KD of between about 0.1 nM to about 40 nM, between about 0.5 nM to about 30 nM, between about 1 nM to about 20 nM, or between about 1.5 nM to about 20 nM.

In a preferable aspect, the antibody or antigen binding fragment thereof binds to a B7-H4 (preferably a human B7-H4) with a KD of between about 23 nM to about 27 nM. In a more preferable aspect, the antibody or antigen binding fragment thereof binds to a B7-H4 (preferably a human B7-H4) with a KD of between about 1 nM to about 1.5 nM.

The KD measurements (binding affinity) may be carried out by any suitable assay known in the art. Suitable assays include an affinity assay performable via a KinExA system (e.g., KinExA 3100, KinExA 3200, or KinExA 4000) (Sapidyne Instruments, Idaho), or ForteBio Octet system.

In some aspects, the extent of binding of an antibody or antigen binding fragment thereof of the disclosure to an unrelated, non-B7-H4 protein is less than about 10%, 5%, 2% or 1% (preferably less than about 10%) of the binding of the antibody (or antigen binding fragment thereof) to B7-H4 (preferably human B7-H4). Said binding may be measured, e.g., by a radioimmunoassay (RIA), BIACORE® (using recombinant B7-H4 as the analyte and antibody as the ligand, or vice versa), KINEXA®, ForteBio Octet system, or other binding assays known in the art.

In some aspects, the antibody or antigen binding fragment thereof does not bind to one or more selected from a human B7-H1 molecule, a human B7-H2 molecule, a human B7-H3 molecule, a human BTN1A1 molecule, a human HHLA2 molecule, a human BTN3A2 molecule, or a combination thereof. In a preferable aspect, the antibody or antigen binding fragment thereof does not bind to one or more selected from a human B7-H1 molecule, a human B7-H2 molecule, a human B7-H3 molecule, or a combination thereof.

The term “does not bind” means that the antibody or antigen binding fragment thereof described herein does not substantially bind to one of more of said molecules (e.g., human B7-H1 molecule, a human B7-H2 molecule, a human B7-H3 molecule, a human BTN1A1 molecule, a human HHLA2 molecule, a human BTN3A2 molecule, or a combination thereof). The term “substantially no” when used in the context of binding herein may mean less than 5%, 2%, 1%, 0.5% or 0.1% of cells expressing one or more of said molecules in a cell culture become bound by the antibody or antigen binding fragment thereof described herein (upon contact therewith). Suitably, the term “substantially no” when used in the context of binding herein may mean no such cells become bound.

In some aspects, the antibody or antigen binding fragment thereof does not bind to a human B7-H1 molecule, a human B7-H2 molecule, a human B7-H3 molecule, a human BTN1A1 molecule, a human HHLA2 molecule, or a human BTN3A2 molecule. In a preferable aspect, the antibody or antigen binding fragment thereof does not bind to a human B7-H1 molecule, a human B7-H2 molecule, or a human B7-H3 molecule.

In some aspects, the B7-H4 polypeptide is comprised within a B7-H4 polypeptide sequence, or a fragment thereof.

A “B7-H4 polypeptide” may comprise the full length polypeptide sequence of B7-H4 (e.g., SEQ ID NO.: 55), or may comprise a fragment of B7-H4 of any length of the full length polypeptide sequence of B7-H4 (e.g., comprising a polypeptide sequence of 5%, 15%, 25%, 35%, 45%, 55%, 65%, 75%, 85% or 95% of the full length polypeptide sequence of B7-H4) which comprises an epitope which can bind (e.g. be bound by) an antibody or antigen binding fragment of the disclosure. The B7-H4 polypeptide may comprise a sequence having 75%, 80%, 85%, 90% or 90% sequence identity to the sequence of SEQ ID NO.: 55. Preferably, the B7-H4 polypeptide comprises the sequence of SEQ ID NO.: 55.

### Characteristics of Antibodies and Antigen Binding Fragments

The antibody or antigen binding fragment has high affinity for B7-H4 both in vitro an in vivo, and thus may advantageously be used in methods for detecting a B7-H4 epitope, and associated methods of diagnosis.

The term “antibody” covers monoclonal antibodies and fragments thereof (e.g., exhibiting the desired biological activity). In a preferable aspect, an antibody of the present disclosure is a monoclonal antibody. In a more preferable aspect, the antibody is a fully human monoclonal antibody. In some aspects, methods of the disclosure may employ polyclonal antibodies.

In particular, an antibody is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al, J. MoI. Biol. 196:901-917, 1987). Preferably, each VHC and VLC is composed of three CDRs and four FRs, arranged from amino-terminus to carboxy-terminus in the following order: FR1, CDR1, FR2, DR2, FR3, CDR3, FR4. The VHC or VLC chain of the antibody can further include all or part of a heavy or light chain constant region. In some aspects, the antibody is a tetramer of two heavy immunoglobulin chains and two light immunoglobulin chains, wherein the heavy and light immunoglobulin chains are interconnected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The term “antibody” includes intact immunoglobulins of types IgA, IgG, IgE, IgD, IgM (as well as subtypes thereof), wherein the light chains of the immunoglobulin may be of types kappa or lambda. The term antibody, as used herein, also refers to a portion of an antibody that binds to one of the above-mentioned markers, e.g., a molecule in which one or more immunoglobulin chains is not full length, but which binds to a marker. Examples of binding portions encompassed within the term antibody include (i) a Fab fragment, a monovalent fragment consisting of the VLC, VHC, CL and CH1 domains; (ii) a F(ab′)2 fragment, a bivalent fragment comprising two Fab fragments linked by a disulfide bridge at the hinge region; (iii) a Fc fragment consisting of the VHC and CH1 domains; (iv) a Fv fragment consisting of the VLC and VHC domains of a single arm of an antibody, (v) a dAb fragment (Ward et al, Nature 341:544-546, 1989), which consists of a VHC domain; and (vi) an isolated complementarity determining region (CDR) having sufficient framework to bind, e.g. an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science 1Al-ATi-Alβ; and Huston et al. (1988) Proc. Natl. Acad. ScL USA 85:5879-5883). Such single chain antibodies are also encompassed within the term antibody. These may be obtained using conventional techniques known to those skilled in the art, and the portions are screened for utility in the same manner as are intact antibodies.

In some aspects, the antibody or antigen binding fragment is one or more selected from a murine antibody, a humanized antibody, a chimeric antibody, a monoclonal antibody, a polyclonal antibody, a recombinant antibody, a multispecific antibody, or a combination thereof.

In some aspects, the antigen-binding fragment is one or more selected from a Fv fragment, an Fab fragment, an F(ab′)2 fragment, an Fab′ fragment, a dsFv fragment, an scFv fragment, an sc(Fv)2 fragment, or a combination thereof.

In some aspects, the antibody or antigen binding fragment thereof (e.g., mAb) of the disclosure is a scFV.

In some aspects, the antibody or antigen binding fragment thereof can bind to B37-H4 molecules across species, e.g., the antibody or fragment can bind to mouse B7-H4, rat B7-H4, rabbit, human B7-H4 and/or cynomolgus monkey B7-H4. In some aspects, the antibody or fragment can bind to human B7-H4 and cynomolgus monkey B7-H4. In some aspects, the antibody or antigen binding fragment can also bind to mouse B7-H4.

In some aspects, the antibody or antigen binding fragment thereof can specifically bind to B7-H4, e.g., human B7-H4 and cynomolgus monkey B7-H4, but does not specifically bind to human B7-H1, B7-H2, and/or B7-H3.

In some aspects, the antibody or antigen-binding fragment thereof can include, in addition to a VH and a VL, a heavy chain constant region or fragment thereof. In some aspects, the heavy chain constant region is a human heavy chain constant region, e.g., a human IgG constant region, e.g., a human IgG1 constant region. In some aspects (preferably where the antibody or antigen-binding fragment thereof is conjugated to an agent, such as a cytotoxic agent), a cysteine residue is inserted between amino acid S239 and V240 in the CH2 region of IgG1. This cysteine is referred to as “a 239 insertion” or “239i.”

In some aspects, the antibody or antigen binding fragment thereof may comprise a heavy chain constant region comprising the amino acid sequence of SEQ ID NO: 41. Preferably, the antibody or antigen binding fragment thereof may comprise a heavy chain constant region comprising the amino acid sequence of SEQ ID NO: 52.

The “E02-GL” antibody has the CDR sequences (e.g. corresponds to) of ZYOEQD-E02 (“GL” means the antibody has been germlined). For example, E02-GL may comprise the VH chain sequence of SEQ ID NO: 45, e.g. a germlined version of SEQ ID NO: 43, and the VL chain sequence of SEQ ID NO: 34. E02-GL may also comprise the heavy chain sequence of SEQ ID NO: 51 and the light chain sequence of SEQ ID NO: 44. “E02-GL” conjugated to the toposiomersase I payload SG3932 as described herein is therefore referred to as “E02-GL-SG3932”. In one aspect, the clone E02-GL is conjugated to the topoisomerase I payload SG3932 at an average Drug Antibody Ratio (DAR) of 8.

In some aspects, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In some aspects the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. In a preferable aspect, the IgG constant domain can contain amino acid substitutions relative to a wild-type human IgG constant domain including as substitution of the amino acid at Kabat position 252 with Tyrosine (Y), a substitution of the amino acid at Kabat position 254 with Threonine (T), and a substitution of the amino acid at Kabat position 256 with Glutamic acid (E). In some aspects, the antibody or antigen-binding fragment thereof comprises a heavy chain, wherein the heavy chain is a human IgG1 YTE mutant.

In some aspects, the antibody or antigen-binding fragment thereof can include, in addition to a VH and a VL, and optionally a heavy chain constant region or fragment thereof, a light chain constant region or fragment thereof. In some aspects, the light chain constant region is a kappa lambda light chain constant region, e.g., a human kappa constant region or a human lambda constant region.

In some aspects, the antibody or antigen binding fragment thereof comprises a light chain constant region comprising the amino acid sequence of SEQ ID NO: 42.

In some aspects, a VH and/or VL amino acid sequence can have 85%, 90%, 95%, 96%, 97%, 98% or 99% similarity to a sequence set forth herein. In some aspects, a VH and/or VL amino acid sequence may comprise 1, 2, 3, 4, 5 or more substitutions, e.g., conservative substitutions relative to a sequence set forth herein. A B7-H4 antibody having VH and VL regions having a certain percent similarity to a VH region or VL region, or having one or more substitutions, e.g., conservative substitutions can be obtained by mutagenesis (e.g., site-directed or PCR-mediated mutagenesis) of nucleic acid molecules encoding VH and/or VL regions described herein, followed by testing of the encoded altered antibody for binding to B7-H4 and optionally testing for retained function using the functional assays described herein.

The affinity or avidity of an antibody or antigen binding fragment thereof for an antigen can be determined experimentally using any suitable method well known in the art, e.g., flow cytometry, enzyme-linked immunosorbent assay (ELISA), or radioimmunoassay (RIA), or kinetics (e.g., KINEXA® or BIACORE™ analysis). Direct binding assays as well as competitive binding assay formats can be readily employed. (See, e.g., Berzofsky et al., Antibody-Antigen Interactions, In Fundamental Immunology, Paul, W. E., Ed., Raven Press: New York, N.Y. (1984); Kuby, Immunology, W. H. Freeman and Company: New York, N.Y. (1992); and methods described herein.) The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art.

In some aspects, the antibody or antigen-binding fragment thereof, can bind to B7-H4-expressing cells with an IC50 lower than about 500 nM, lower than about 350 nM, lower than about 250 nM, lower than about 150 nM, lower than about 100 nM, lower than about 75 nM, lower than about 60 nM, lower than about 50 nM, lower than about 40 nM, lower than about 30 nM, lower than about 20 nM, lower than about 15 nM, lower than about 10 nM, lower than about 5 nM, lower than about 1 nM, lower than about 500 pM, lower than about 350 pM, lower than about 250 pM, lower than about 150 pM, lower than about 100 pM, lower than about 75 pM, lower than about 60 pM, lower than about 50 pM, lower than about 40 pM, lower than about 30 pM, lower than about 20 pM, lower than about 15 pM, lower than about 10 pM, or lower than about 5 pM. Preferably, said IC50 is measured by flow cytometry.

### Cleavable Linkers and Cytotoxic Agents

In some aspects of the antibody-drug conjugates disclosed herein, the antibody or antigen binding fragment thereof is linked to a cytotoxic agent by a linker. In some aspects, the antibody or antigen binding fragment thereof is conjugated to a cytotoxic agent by a linker. As used herein, “conjugated” means linked via a covalent or ionic bond. The cytotoxic agent may be referred to herein as an “agent” or “active agent.” In some aspects, the cytotoxic agent is a drug.

In some aspects, the cytotoxic agent on the ADC (also sometimes referred to as a “warhead”) is one of the cytotoxic agents disclosed in published international application WO2020/200880, incorporated by reference herein in its entirety.

The cytotoxic agent or cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. These include, but are not limited to, topoisomerase I inhibitors, amanitins, auristatins, daunomycins, doxorubicins, duocarmycins, dolastatins, enediynes, lexitropsins, taxanes, puromycins, maytansinoids, vinca alkaloids, tubulysins and pyrrolobenzodiazepines (PBDs). Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof. Additional disclosure regarding cytotoxins suitable for use in ADCs can be found, for example, in International Patent Application Publication Nos. WO 2015/155345 and WO 2015/157592, incorporated by reference herein in their entirety.

In some aspects, the antibody or antigen-binding fragment is conjugated to one or more heterologous agent selected from the group consisting of a topoisomerase I inhibitor, a tubulysin derivative, a pyrrolobenzodiazepine, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

In some aspects, the antibody antigen binding fragment is conjugated to one or more cytotoxin selected from a topoisomerase I inhibitor, tubulysin derivative, a pyrrolobenzodiazepine, or a combination thereof. For example, the antibody or antigen binding fragment thereof is conjugated to one or more cytotoxin selected from the group consisting of topoisomerase I inhibitor SG3932, SG4010, SG4057 or SG4052 (the structures of which are provided below); tubulysin AZ1508, pyrrolobenzodiapezine SG3315, pyrrolobenzodiapezine SG3249, or a combination thereof.

In preferred aspects, the antibody or antigen binding fragment thereof is conjugated to a topoisomerase I inhibitor. Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle.

A general example of a suitable topoisomerase I inhibitor is represented by the following compound A*:

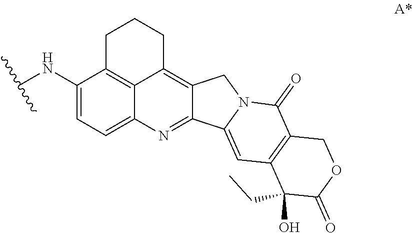 [SMILES: *Nc1ccc2nc3c(c4c2c1CCC4)Cn1c-3cc2c(c1=O)COC(=O)[C@]2(O)CC]

which may be referred to as a “Drug Unit” herein.

The compound (e.g., A*) is preferably provided with a linker for connecting (preferably conjugating) to an antibody or antigen binding fragment described herein (which may be referred to as a “Ligand Unit”). Suitably, the linker is attached (e.g., conjugated) in a cleavable manner to an amino residue, for example, an amino acid of an antibody or antigen binding fragment described herein.

More particularly, an example of a suitable topoisomerase I inhibitor is represented by the following compound, with the formula “I”:

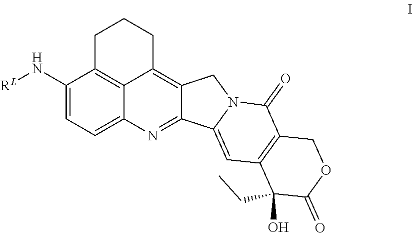

and salts and solvates thereof, wherein R^{L} is a linker for connection to an antibody or antigen binding fragment thereof described herein (e.g., the Ligand Unit), wherein said linker is preferably selected from:
(ia):

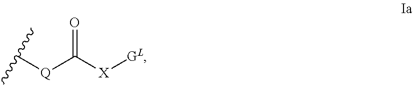

wherein
Qis:

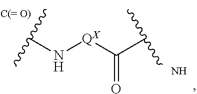

where Q^{X} is such that Q is an amino-acid residue, a dipeptide residue, a tripeptide residue or a tetrapeptide residue;
X is:

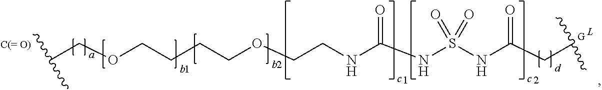

where a=0 to 5, b1=0 to 16, b2=0 to 16, c1=0 or 1, c2=0 or 1, d=0 to 5, wherein at least b1 or b2=0 (i.e., only one of b1 and b2 may not be 0) and at least c1 or c2=0 (i.e., only one of c1 and c2 may not be 0);
G^{L} is a linker for connecting to an antibody or antigen binding fragment thereof described herein (e.g., the Ligand Unit); or
(ib):

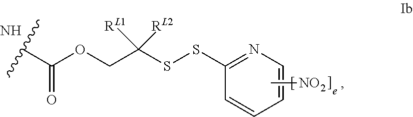

where R^{L1} and R^{L2} are independently selected from H and methyl, or together with the carbon atom to which they are bound form a cyclopropylene or cyclobutylene group; and e is 0 or 1.

It will be understood by the person skilled in the art that more than one of said agent(s) (e.g., topoisomerase I inhibitor) may be conjugated to the antibody or antigen binding fragment thereof.

For example, a conjugate (e.g., antibody-drug conjugate) of the disclosure may be of the general formula IV:

L−(D^{L})ₚ (IV)

or a pharmaceutically acceptable salt or solvate thereof, wherein L is an antibody or antigen binding fragment thereof described herein (e.g., the Ligand Unit), D^{L} is a topoisomerase I inhibitor having a linker (e.g., Drug Linker unit) that is of formula III:

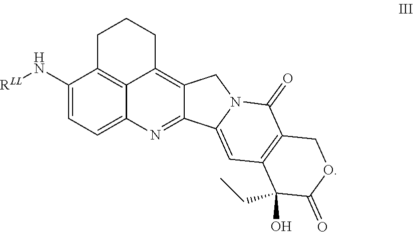

R^{LL} is a linker connected to an antibody or antigen binding fragment thereof described herein (e.g., the Ligand Unit), wherein the linker is preferably selected from
(ia′):

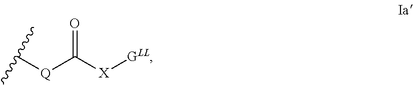

where Q and X are as defined above and G^{LL} is a linker connected to an antibody or antigen binding fragment thereof described herein (e.g., the Ligand Unit); and
(ib′):

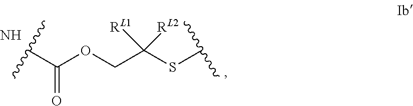

where R^{L1} and R^{L2} are as defined above; and
p is an integer of from 1 to 20.

The drug loading is represented by p, the number of topoisomerase I inhibitor(s) (e.g., Drug units) per antibody or antigen binding fragment thereof (e.g., Ligand Unit). Drug loading may range from 1 to 20 Drug units (D) per Ligand unit. For compositions, p represents the average drug loading of the conjugates in the composition, and p ranges from 1 to 20.

Accordingly, aspects of the ADCs disclosed herein encompass a conjugate comprising an antibody or antigen binding fragment thereof described herein (e.g., the Ligand Unit) covalently linked to at least one topoisomerase I inhibitor (e.g., Drug unit, such as A* illustrated above). Said inhibitor is preferably linked to the antibody or antigen binding fragment thereof by a linker (e.g., Linker unit), such as a linker described above as R^{L} and/or R^{LL}. In other words, aspects of the ADCs disclosed herein encompass an antibody or antigen binding fragment thereof described herein (e.g., the Ligand Unit) with one or more topoisomerase I inhibitors attached, preferably via a linker (e.g., Drug-Linker units). The antibody or antigen binding fragment thereof (representing a Ligand unit), described more fully above, is a targeting agent that binds to a target moiety. More particularly, this Ligand unit can, for example, specifically bind to a B7-H4 on a target cell, to which the Drug unit is thus delivered. Accordingly, the methods described herein can be used for the treatment of, for example, various cancers and other disorders with an ADC (e.g., cancers/disorders which are associated with the presence of cells, preferably cancerous cells, which express B7-H4), as discussed elsewhere herein.

Certain features of the topoisomerase I inhibitors described above are particularly preferred and may be defined in more detail as set out below. By way of example, a preferred aspect of feature Q^{X} (e.g., within the linker of 1a described above) will be outlined.

The following preferences may apply to all aspects of the methods, combinations and kits as described herein, or may relate to a single aspect. The preferences may be combined together in any combination.

Q^{X}

In some aspects, Q is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In some aspects, Q comprises a dipeptide residue. The amino acids in the dipeptide may be any combination of natural amino acids and non-natural amino acids. In some aspects, the dipeptide comprises natural amino acids. Where the linker is a cathepsin labile linker, the dipeptide is the site of action for cathepsin-mediated cleavage. The dipeptide then is a recognition site for cathepsin.

In some aspects, Q is selected from:
^{NH}-Phe-Lys-^{C=O},
^{NH}-Val-Ala-^{C=O},
^{NH}-Val-Lys-^{C=O},
^{NH}-Ala-Lys-^{C=O},
^{NH}-Val-Cit-^{C=O},
^{NH}-Phe-Cit-^{C=O},
^{NH}-Leu-Cit-^{C=O},
^{NH}-Ile-Cit-^{C=O},
^{NH}-Phe-Arg-^{C=O},
^{NH}-Trp-Cit-^{C=O}, and
^{NH}-Gly-Val-^{C=O},
where Cit is citrulline.

Preferably, Q is selected from:
^{NH}-Phe-Lys-^{C=O},
^{NH}-Val-Ala-^{C=O},
^{NH}-Val-Lys-^{C=O},
^{NH}-Ala-Lys-^{C=O}, and
^{NH}-Val-Cit-^{C=O}.

More preferably, Q is selected from ^{NH}-Phe-Lys-^{C=O}, ^{NH}-Val-Cit-^{C=O} or ^{NH}-Val-Ala-^{C=O.}

Other suitable dipeptide combinations include:
^{NH}-Gly-Gly-^{C=O},
^{NH}-Gly-Val-^{C=O}
^{NH}-Pro-Pro-^{C=O}, and
^{NH}-Val-Glu-^{C=O}.

Other dipeptide combinations may be used, including those described by Dubowchik et al., Bioconjugate Chemistry, 2002, 13,855-869, which is incorporated herein by reference.

In some aspects, Q is a tripeptide residue. The amino acids in the tripeptide may be any combination of natural amino acids and non-natural amino acids. In some aspects, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-^{C=O}
^{NH}-Glu-Val-Cit-^{C=O}
^{NH}-αGlu-Val-Ala-^{C=O}
^{NH}-αGlu-Val-Cit-^{C=O}

In some aspects, Q is a tetrapeptide residue. The amino acids in the tetrapeptide may be any combination of natural amino acids and non-natural amino acids. In some aspects, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:
^{NH}-Gly-Gly-Phe-Gly^{C=O}; and
^{NH}-Gly-Phe-Gly-Gly^{C=O.}

In some aspects, the tetrapeptide is:
^{NH}-Gly-Gly-Phe-Gly^{C=O.}

In the above representations of peptide residues, ^{NH}-represents the N-terminus, and —^{C=O} represents the C-terminus of the residue. The C-terminus binds to the NH of A*.

Glu represents the residue of glutamic acid, i.e.:

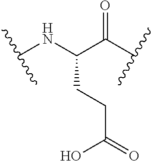 [SMILES: *N[C@@H](CCC(=O)O)C(*)=O]

αGlu represents the residue of glutamic acid when bound via the α-chain, i.e.:

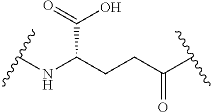 [SMILES: *N[C@@H](CCC(*)=O)C(=O)O]

In some aspects, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

G^{L}

G^{L} may be selected from:

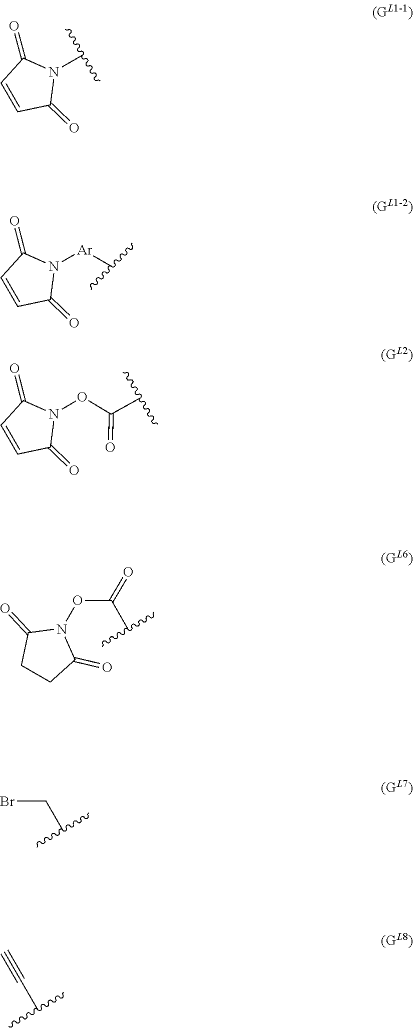

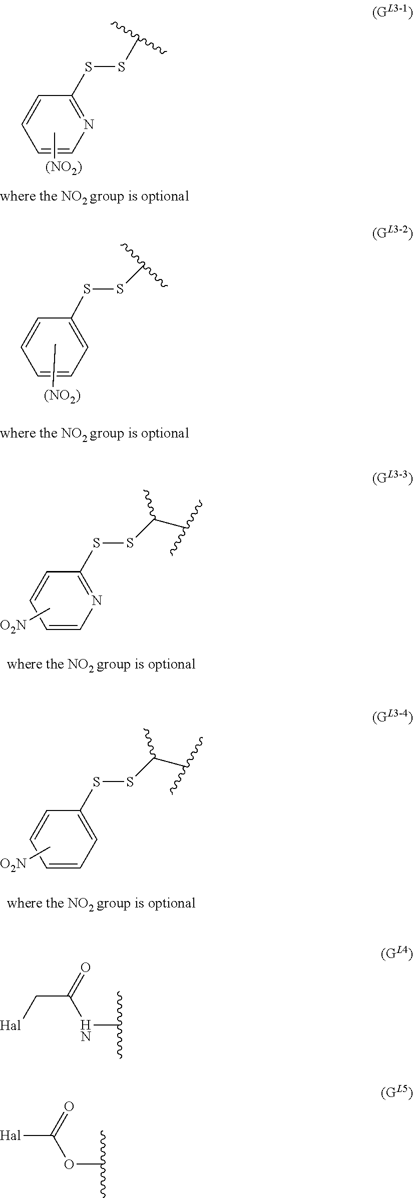

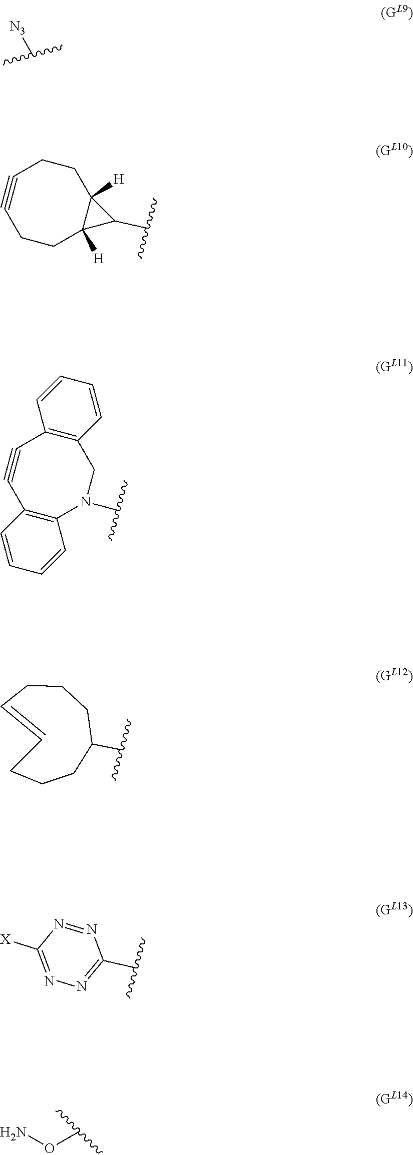

where Ar represents a C₅₋₆ arylene group, e.g., phenylene, and X represents C₁₋₄ alkyl.

In some aspects, G^{L} is selected from G^{L1-1} and G^{L1-2}. In some of these aspects, G^{L} is G^{L1-1}.

G^{LL}

G^{LL} may be selected from:

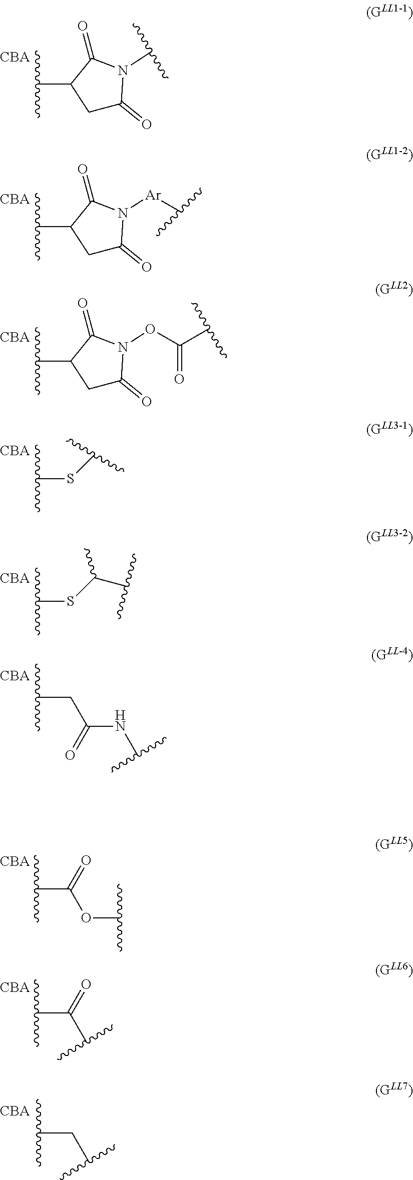

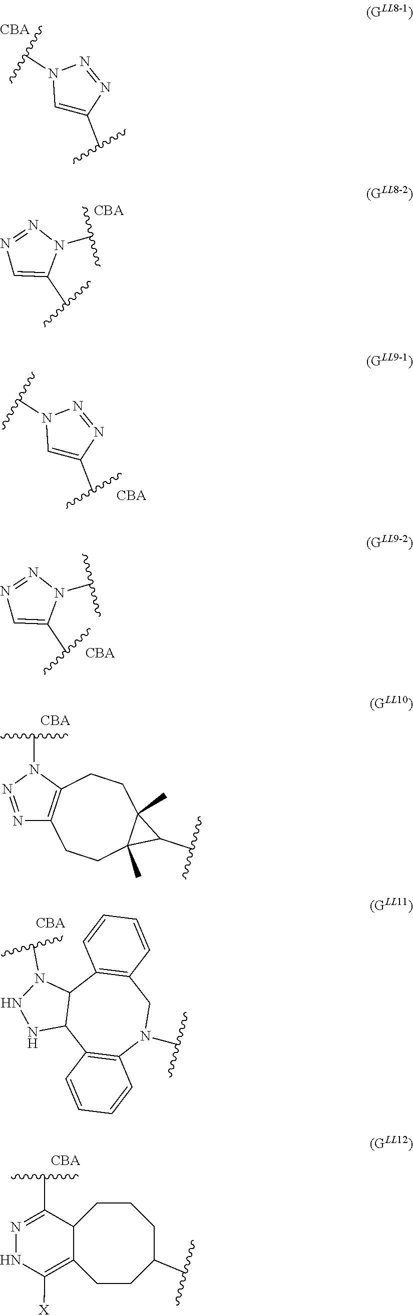

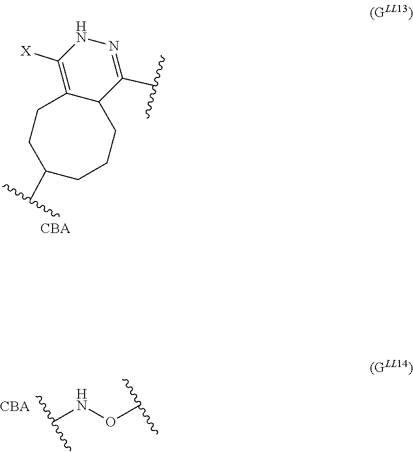

where Ar represents a C₅₋₆ arylene group, e.g., phenylene and X represents C₁₋₄ alkyl.

In some aspects, G^{LL} is selected from G^{LL1-1} and G^{LL1-2}. In some of these aspects, G^{LL} is G^{LL1-1}.

X

X is preferably:

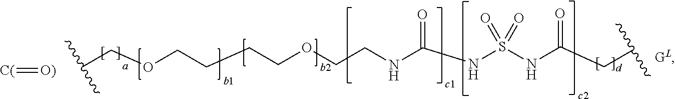

where a=0 to 5, b1=0 to 16, b2=0 to 16, c=0 or 1, d=0 to 5, wherein at least b1 or b2=0 and at least c1 or c2=0.
a may be 0, 1, 2, 3, 4 or 5. In some aspects, a is 0 to 3. In some of these aspects, a is 0 or 1. In further aspects, a is 0.
b1 may be 0, 1, 2, 3, 4, 5, 6, 7, 8, 9, 10, 11, 12, 13, 14, 15 or 16. In some aspects, b1 is 0 to 12. In some of these aspects, b1 is 0 to 8, and may be 0, 2, 3, 4, 5 or 8.
b2 may be 0, 1, 2, 3, 4, 5, 6, 7, 8, 9, 10, 11, 12, 13, 14, 15 or 16. In some aspects, b2 is 0 to 12. In some of these aspects, b2 is 0 to 8, and may be 0, 2, 3, 4, 5 or 8. Preferably, only one of b1 and b2 may not be 0.
c1 may be 0 or 1. c2 may be 0 or 1. Preferably, only one of c1 and c2 may not be 0.
d may be 0, 1, 2, 3, 4 or 5. In some aspects, d is 0 to 3. In some of these aspects, d is 1 or 2. In further aspects, d is 2. In further aspects, d is 5.

In some aspects of X, a is 0, b1 is 0, c1 is 1, c2 is 0 and d is 2, and b2 may be from 0 to 8. In some of these aspects, b2 is 0, 2, 3, 4, 5 or 8. In some aspects of X, a is 1, b2 is 0, c1 is 0, c2 is 0 and d is 0, and b1 may be from 0 to 8. In some of these aspects, b1 is 0, 2, 3, 4, 5 or 8. In some aspects of X, a is 0, b1 is 0, c1 is 0, c2 is 0 and d is 1, and b2 may be from 0 to 8. In some of these aspects, b2 is 0, 2, 3, 4, 5 or 8. In some aspects of X, b1 is 0, b2 is 0, c1 is 0, c2 is 0 and one of a and d is 0. The other of a and d is from 1 to 5. In some of these aspects, the other of a and d is 1. In other of these aspects, the other of a and d is 5. In some aspects of X, a is 1, b2 is 0, c1 is 0, c2 is 1, d is 2, and b1 may be from 0 to 8. In some of these aspects, b2 is 0, 2, 3, 4, 5 or 8.

In some aspects, R^{L} is of formula Ib. In some aspects, R^{LL} is formula Ib′.

R^{L1} and R^{L2} may be independently selected from H and methyl, or together with the carbon atom to which they are bound form a cyclopropylene or cyclobutylene group.

In some aspects, both R^{L1} and R^{L2} are H. In some aspects, R^{L1} is H and R^{L2} is methyl. In some aspects, both R^{L1} and R^{L2} are methyl.

In some aspects, R^{L1} and R^{L2} together with the carbon atom to which they are bound form a cyclopropylene group. In some aspects, R^{L1} and R^{L2} together with the carbon atom to which they are bound form a cyclobutylene group.

In the group Ib, in some aspects, e is 0. In other aspects, e is 1 and the nitro group may be in any available position of the ring. In some of these aspects, it is in the ortho position. In others of these aspects, it is in the para position.

In some aspects where compounds described herein are provided in a single enantiomer or in an enantiomerically enriched form, the enantiomerically enriched form has an enantiomeric ratio greater than 60:40, 70:30; 80:20 or 90:10. In further aspects, the enantiomeric ratio is greater than 95:5, 97:3 or 99:1.

In some aspects, R^{L} is selected from:

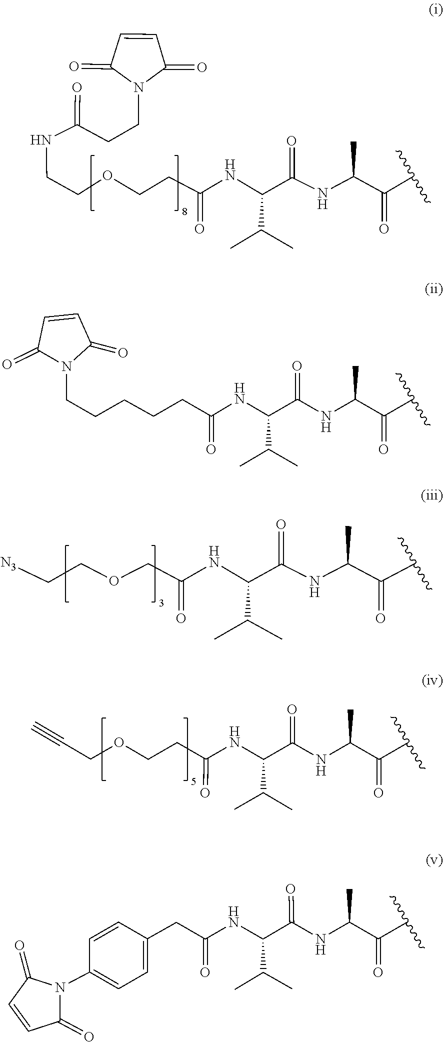

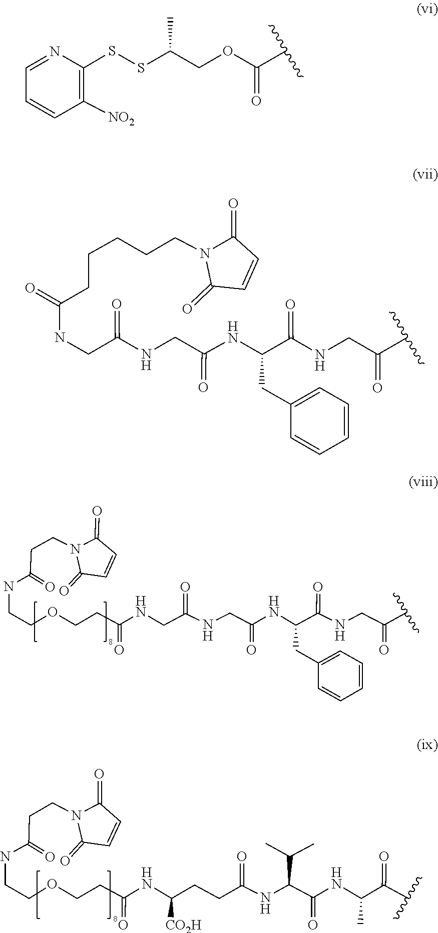

In some aspects, R^{LL} is a group derived from the R^{L} groups above.

Having outlined said preferences above, certain preferred topoisomerase I-linker (e.g., Drug Linker unit) formulas are now described.

In some aspects, the compound of formula I is of the formula I^{P}:

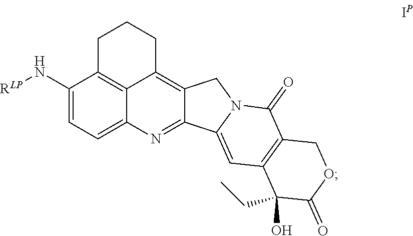

and salts and solvates thereof, wherein R^{LP} is a linker for connection to an antibody or antigen binding fragment thereof described herein, wherein said linker is selected from:
(ia):

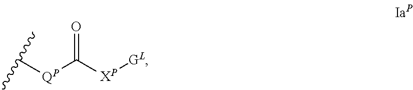

wherein
Q^{P} is:

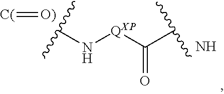

where Q^{XP} is such that Q^{P} is an amino-acid residue, a dipeptide residue or a tripeptide residue;
X is:

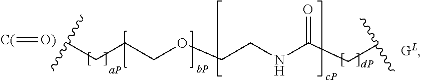

where aP=0 to 5, bP=0 to 16, cP=0 or 1, dP=0 to 5;
G^{L} is a linker for connecting to an antibody or antigen binding fragment thereof described herein (e.g., Ligand Unit);
(ib):

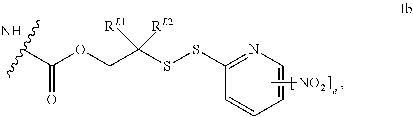

where R^{L1} and R^{L2} are independently selected from H and methyl, or together with the carbon atom to which they are bound form a cyclopropylene or cyclobutylene group; and
e is 0 or 1.
aP may be 0, 1, 2, 3, 4 or 5. In some aspects, aP is 0 to 3. In some of these aspects, aP is 0 or 1. In further aspects, aP is 0.
bP may be 0, 1, 2, 3, 4, 5, 6, 7, 8, 9, 10, 11, 12, 13, 14, 15 or 16. In some aspects, b is 0 to 12. In some of these aspects, bP is 0 to 8, and may be 0, 2, 4 or 8.
cP may be 0 or 1.
dP may be 0, 1, 2, 3, 4 or 5. In some aspects, dP is 0 to 3. In some of these aspects, dP is 1 or 2. In further aspects, dP is 2.

In some aspects of X, aP is 0, cP is 1 and dP is 2, and bP may be from 0 to 8. In some of these aspects, bP is 0, 4 or 8.

The preferences for Q^{X} above for compounds of Formula I may apply to Q^{XP} (for example, where appropriate).

The preferences for G^{L}, R^{L1}, R^{L2} and e above for compounds of Formula I may apply to compounds of Formula I^{P}.

In some aspects, the conjugate of formula IV is of the formula IV^{P}:

L−(D^{LP})_{P} (IV^{P})

or a pharmaceutically acceptable salt or solvate thereof, wherein L is an antibody or antigen binding fragment thereof described herein (e.g., Ligand Unit), D^{LP} is a topoisomerase I inhibitor (e.g., Drug Linker unit) that is of formula III^{P}:

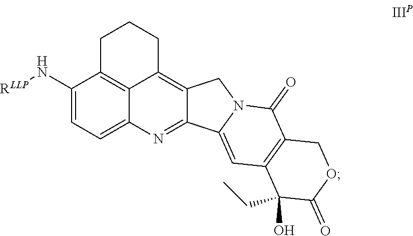

R^{LLP} is a linker connected to the antibody or antigen binding fragment thereof (e.g., Ligand unit), wherein said linker is selected from
(ia′):

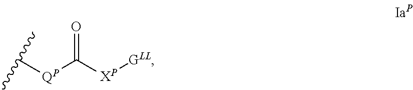

where Q^{P} and X are as defined above and G^{LL} is a linker connected to an antibody or antigen binding fragment thereof described herein (e.g., Ligand Unit); and
(ib′):

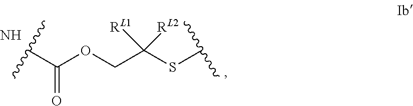

where R^{L1} and R^{L2} are as defined above; and p is an integer of from 1 to 20.

In some aspects, the compound of formula I is of the formula I^{P2}:

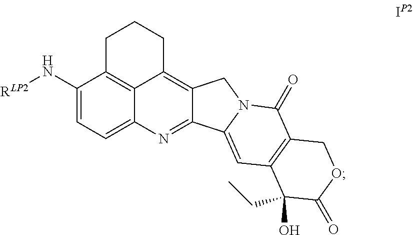

and salts and solvates thereof, wherein R^{LP2} is a linker for connection to an antibody or antigen binding fragment thereof described herein, wherein said linker is selected from:
(ia):

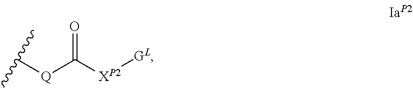

wherein
Q is:

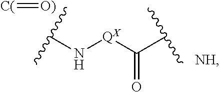

where Q^{X} is such that Q is an amino-acid residue, a dipeptide residue, a tripeptide residue or a tetrapeptide residue;
X^{P2} is:

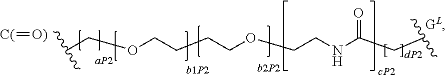

where aP2=0 to 5, b1P2=0 to 16, b2P2=0 to 16, cP2=0 or 1, dP2=0 to 5, wherein at least b1P2 or b2P2=0 (i.e., only one of b1 and b2 may not be 0);
G^{L} is a linker for connecting to an antibody or antigen binding fragment thereof described herein (e.g., Ligand Unit);
(ib):

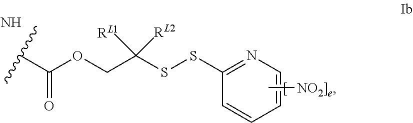

where R^{L1} and R^{L2} are independently selected from H and methyl, or together with the carbon atom to which they are bound form a cyclopropylene or cyclobutylene group; and
e is 0 or 1.

aP2 may be 0, 1, 2, 3, 4 or 5. In some aspects, aP2 is 0 to 3. In some of these aspects, aP2 is 0 or 1. In further aspects, aP2 is 0.

b1P2 may be 0, 1, 2, 3, 4, 5, 6, 7, 8, 9, 10, 11, 12, 13, 14, 15 or 16. In some aspects, b1P2 is 0 to 12. In some of these aspects, b1P2 is 0 to 8, and may be 0, 2, 3, 4, 5 or 8.

b2P2 may be 0, 1, 2, 3, 4, 5, 6, 7, 8, 9, 10, 11, 12, 13, 14, 15 or 16. In some aspects, b2P2 is 0 to 12. In some of these aspects, b2P2 is 0 to 8, and may be 0, 2, 3, 4, 5 or 8.

Preferably, only one of b1P2 and b2P2 may not be 0.

cP2 may be 0 or 1.

dP2 may be 0, 1, 2, 3, 4 or 5. In some aspects, dP2 is 0 to 3. In some of these aspects, dP2 is 1 or 2. In further aspects, dP2 is 2. In further aspects, dP2 is 5.

In some aspects of X², aP2 is 0, b1P2 is 0, cP2 is 1 and dP2 is 2, and b2P2 may be from 0 to 8. In some of these aspects, b2P2 is 0, 2, 3, 4, 5 or 8. In some aspects of X², aP2 is 1, b2P2 is 0, cP2 is 0 and dP2 is 0, and b1P2 may be from 0 to 8. In some of these aspects, b1P2 is 0, 2, 3, 4, 5 or 8. In some aspects of X², aP2 is 0, b1P2 is 0, cP2 is 0 and dP2 is 1, and b2P2 may be from 0 to 8. In some of these aspects, b2P2 is 0, 2, 3, 4, 5 or 8. In some aspects of X², b1P2 is 0, b2P2 is 0, cP2 is 0 and one of aP2 and dP2 is 0. The other of aP2 and d is from 1 to 5. In some of these aspects, the other of aP2 and d is 1. In other of these aspects, the other of aP2 and dP2 is 5.

The preferences for Q^{X} above for compounds of Formula I may apply to Q^{X} in Formula Ia^{P2} (e.g., where appropriate).

The preferences for G^{L}, R^{L1}, R^{L2} and e above for compounds of Formula I may apply to compounds of Formula I^{P2}.

In some aspects, the conjugate of formula IV is of the formula IV^{P2}:

L−(D^{LP2})_{P} (IV^{P2})

or a pharmaceutically acceptable salt or solvate thereof, wherein L is an antibody or antigen binding fragment thereof described herein (e.g., Ligand unit), D^{LP2} is a topoisomerase I inhibitor (e.g., Drug Linker unit) that is of formula III^{P2}:

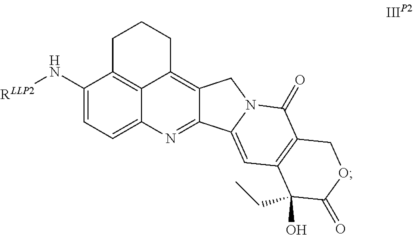

R^{LLP2} is a linker connected to the antibody or antigen binding fragment thereof (e.g., Ligand unit), wherein said linker is selected from
(ia′):

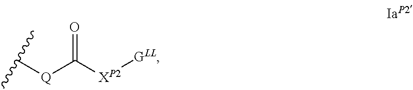

where Q and X² are as defined above and G^{LL} is a linker connected to the antibody or antigen binding fragment thereof, and
(ib′):

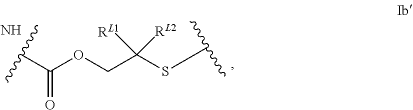

where R^{L1} and R^{L2} are as defined above; and
p is an integer of from 1 to 20.

In some aspects, the linker is an mp-PEG8-val-ala linker. As indicated in the name, the mp-PEG8-val-ala linker has 8 consecutive polyethylene glycol units followed by a valine-alanine (val-ala) dipeptide that attaches to the cytotoxic agent.

In some aspects, the linker and cytotoxic agent together comprise one of the following compounds:

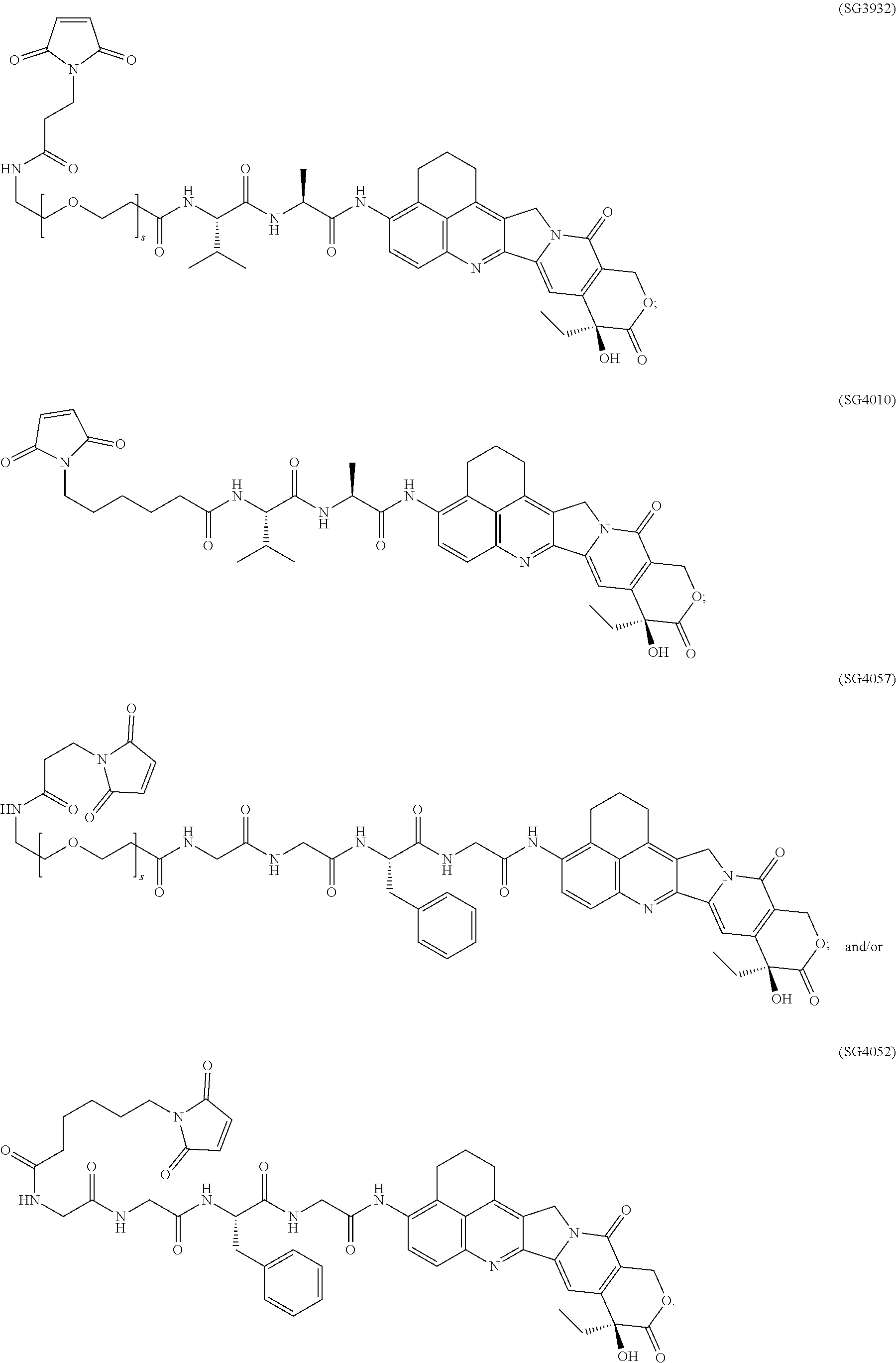

In some aspects, the linker and cytotoxic agent together comprise SG3932:

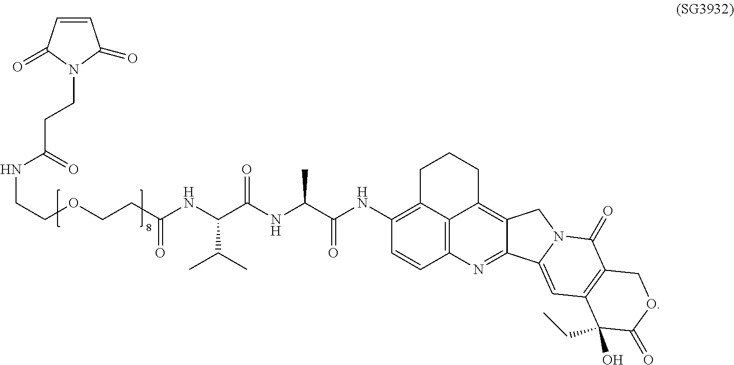

### Drug Antibody Ratio

The agent is typically linked to, or “loaded onto” the antibody or antigen-binding fragment. The agent loading (p) is the average number of agent(s) per antibody or antigen-binding fragment (e.g., the Ligand unit).

The average number of agents per antibody (or antigen-binding fragment) in preparations of ADCs from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). In some instances, separation, purification, and characterization of homogeneous ADC, where p is a certain value from ADC with other drug loadings, may be achieved by means such as reverse phase HPLC or electrophoresis. Such techniques are also applicable to other types of conjugates.

Cysteine amino acids may be engineered at reactive sites in an antibody (or antigen-binding fragment thereof) and which preferably do not form intrachain or intermolecular disulfide linkages (Junutula, et al., 2008b Nature Biotech., 26(8):925-932; Doman et al (2009) Blood 114(13):2721-2729; U.S. Pat. Nos. 7,521,541; 7,723,485; WO2009/052249). The engineered cysteine thiols may react with a linker within an agent (e.g., of formula I below) which may have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody or antigen binding fragment thereof reacts with an agent, then the resulting product may be a mixture of ADC compounds with a distribution of agent units attached to an antibody, e.g., 1, 2, 3, etc. Liquid chromatography methods such as polymeric reverse phase (PLRP) and hydrophobic interaction (HIC) may separate compounds in the mixture by agent loading value. Preparations of ADC with a single agent loading value (p) may be isolated.

Thus, the antibody-drug conjugate compositions of the disclosure may include mixtures of antibody-drug conjugates where the antibody or antigen binding fragment thereof has one or more agent moieties and where the agent moieties may be attached to the antibody or antigen binding fragment thereof at various amino acid residues.

In some aspects, the average number of agents per antibody (or antigen-binding fragment thereof) is in the range 1 to 20. In some aspects the range is selected from 1 to 10, 2 to 10, 2 to 8, 2 to 6, and 4 to 10. In some aspects, there is one agent per antibody (or antigen-binding fragment thereof). In some aspects, the number of agents per antibody (or antigen-binding fragment thereof) can be expressed as a ratio of agent (i.e., drug) to antibody. This ratio is referred to as the Drug to Antibody Ratio (DAR).” The DAR is the average number of drugs (i.e., agents) linked to each antibody. In some aspects of the present disclosure, the DAR is in the range 1 to 20. In some aspects the range of DAR is selected from 1 to 10, 2 to 10, 2 to 8, 2 to 6, and 4 to 10. In some aspects, the DAR is between about 1 and about 8. In a particular aspect of the present disclosure, the DAR is about 8. In a particular aspect of the present disclosure, the DAR is 8.

### Administration and Pharmaceutically Acceptable Compounds for ADCs

In some aspects, the ADC is delivered directly to the site of the adverse cellular population (e.g., thereby increasing the exposure of the diseased tissue to the therapeutic agent). In some aspects, the administration is directly to the airway, e.g., by inhalation or intranasal administration.

An ADC described herein may be comprised within a pharmaceutical composition. The pharmaceutical composition may comprise one or more pharmaceutically acceptable excipient(s). In some aspects, a pharmaceutical composition of the disclosure can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 22nd ed., Ed. Lloyd V. Allen, Jr. (2012).

In some aspects, an ADC pharmaceutical composition of the disclosure may be comprised within one or more formulation selected from a capsule, a tablet, an aqueous suspension, a solution, a nasal aerosol, or a combination thereof.

In some aspects, the ADC pharmaceutical composition comprises more than one type of ADC. For example, a pharmaceutical composition may comprise two or more ADCs having different antibodies, antigen-binding fragments, linkers or cytotoxic agent, or different combination thereof.

The term “a pharmaceutically effective amount” of an antibody or antigen-binding fragment means an amount sufficient to achieve effective binding to a target and to achieve a benefit, e.g., to ameliorate symptoms of a disease or condition or to detect a substance or a cell.

In some aspects, a pharmaceutical composition may comprise a buffer (e.g., acetate, phosphate or citrate buffer), a surfactant (e.g., polysorbate), optionally a stabilizer agent (e.g., human albumin), etc.

### Antibody Preparation

The antibodies of the present disclosure can be obtained using conventional techniques known to persons skilled in the art and their utility confirmed by conventional binding studies—an exemplary method is described in Example 2. By way of example, a simple binding assay is to incubate the cell expressing an antigen with the antibody. If the antibody is tagged with a fluorophore, the binding of the antibody to the antigen can be detected by FACS analysis.

Antibodies of the present disclosure can be raised in various animals including mice, rats, rabbits, goats, sheep, monkeys or horses. Antibodies may be raised following immunization with individual capsular polysaccharides, or with a plurality of capsular polysaccharides. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g., Making and Using Antibodies: A Practical Handbook. GC Howard. CRC Books. 2006. ISBN 0849335280). Polyclonal and monoclonal antibodies are often purified using Protein A/G or antigen-affinity chromatography.

The antibody or antigen binding fragment thereof of the disclosure may be prepared as a monoclonal anti-B7-H4 antibody, which can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or an in vitro binding assay, e.g., radioimmunoassay (RIA) or enzyme-linked immunosorbent assay (ELISA), can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid using known methods.

Alternatively, the antibody or antigen binding fragment thereof (e.g., as monoclonal antibodies) can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

The polynucleotide(s) encoding an antibody or an antigen-binding fragment thereof of the disclosure can further be modified in a number of different manners using recombinant DNA technology to generate alternative antibodies. In some aspects, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In some aspects, the antibody or antigen-binding fragment thereof is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Pat. No. 5,750,373.

In some aspects, the antibody or antigen-binding fragment thereof can be selected from a phage library, where that phage library expresses human antibodies, as described, for example, in Vaughan et al., Nat. Biotech. 14:309-314 (1996); Sheets et al., Proc. Natl. Acad. Sci. USA, 95:6157-6162 (1998); Hoogenboom and Winter, J. Mol. Biol. 227:381 (1991); and Marks et al., J. Mol. Biol. 222:581 (1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Molec. Biol. 376:1182-1200 (2008), each of which is incorporated by reference in its entirety.

Affinity maturation strategies and chain shuffling strategies are known in the art and can be employed to generate high affinity human antibodies or antigen-binding fragments thereof. See Marks et al., BioTechnology 10:779-783 (1992), incorporated by reference in its entirety.

In some aspects, the antibody or antigen binding fragment thereof (e.g., a monoclonal antibody) can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate, or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. Suitably, the CDR residues may be directly and most substantially involved in influencing B7-H4 binding. Accordingly, part or all of the non-human or human CDR sequences are preferably maintained while the non-human sequences of the variable and constant regions can be replaced with human or other amino acids.

Antibodies can also optionally be humanized, resurfaced, engineered or human antibodies engineered with retention of high affinity for the antigen B7-H4 and other favourable biological properties. To achieve this goal, humanized (or human) or engineered anti-B7-H4 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as B7-H4. In this way, FW residues can be selected and combined from the consensus and import sequences so that the desired antibody characteristic, such as increased affinity for the target antigen(s), is achieved.

Humanization, resurfacing or engineering of anti-B7-H4 antibodies or antigen-binding fragments thereof of the present disclosure can be performed using any known method, such as but not limited to those described in, Jones et al., Nature 321:522 (1986); Riechmann et al., Nature 332:323 (1988); Verhoeyen et al., Science 239:1534 (1988); Sims et al., J. Immunol. 151: 2296 (1993); Chothia and Lesk, J. Mol. Biol. 196:901 (1987); Carter et al., Proc. Natl. Acad. Sci. USA 89:4285 (1992); Presta et al., J. Immunol. 151:2623 (1993); U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246; each of which is entirely incorporated herein by reference, including the references cited therein.

Anti-B7-H4 humanized antibodies and antigen-binding fragments thereof can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In some aspects, a fragment (e.g., antibody fragment) of the antibody (e.g., anti-B7-H4 antibody) is provided. Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some aspects, anti-B7-H4 antibody fragments are produced recombinantly. Fab, Fv, and scFv antibody fragments can all be expressed in and secreted from E. coli or other host cells, thus allowing the production of large amounts of these fragments. Such anti-B7-H4 antibody fragments can also be isolated from the antibody phage libraries discussed above. The anti-B7-H4 antibody fragments can also be linear antibodies as described in U.S. Pat. No. 5,641,870. Other techniques for the production of antibody fragments will be apparent to the skilled practitioner.

According to the present disclosure, techniques can be adapted for the production of single-chain antibodies specific to B7-H4. See, e.g., U.S. Pat. No. 4,946,778). In addition, methods can be adapted for the construction of Fab expression libraries to allow rapid and effective identification of monoclonal Fab fragments with the desired specificity for B7-H4, or derivatives, fragments, analogs or homologs thereof. See, e.g., Huse et al., Science 246:1275-1281 (1989). Antibody fragments can be produced by techniques known in the art including, but not limited to: F(ab′)2 fragment produced by pepsin digestion of an antibody molecule; Fab fragment generated by reducing the disulfide bridges of an F(ab′)2 fragment; Fab fragment generated by the treatment of the antibody molecule with papain and a reducing agent; or Fv fragments.

In some aspects, an antibody or antigen-binding fragment thereof of the disclosure can be modified in order to increase its serum half-life. This can be achieved, for example, by incorporation of a salvage receptor binding epitope into the antibody or antibody fragment, by mutation of the appropriate region in the antibody or antibody fragment or by incorporating the epitope into a peptide tag that is then fused to the antibody or antibody fragment at either end or in the middle (e.g., by DNA or peptide synthesis), or by YTE mutation. Other methods to increase the serum half-life of an antibody or antigen-binding fragment thereof, e.g., conjugation to a heterologous molecule, such as PEG, are known in the art.

A modified antibody or antigen-binding fragment thereof as provided herein can comprise any type of variable region that provides for the association of the antibody or polypeptide with B7-H4. In this regard, the variable region can comprise or be derived from any type of mammal that can be induced to mount a humoral response and generate immunoglobulins against the desired antigen. As such, the variable region of an anti-B7-H4 antibody or antigen-binding fragment thereof can be, for example, of human, murine, non-human primate (e.g., cynomolgus monkeys, macaques, etc.) or lupine origin. In some aspects, both the variable and constant regions of the modified antibody or antigen-binding fragment thereof are human. In some aspects, the variable regions of a compatible antibody (usually derived from a non-human source) can be engineered or specifically tailored to improve the binding properties or reduce the immunogenicity of the molecule. In this respect, variable regions useful in the present disclosure can be humanized or otherwise altered through the inclusion of imported amino acid sequences.

In some aspects, the variable domains in both the heavy and light chains of an antibody or antigen-binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain some aspects from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art to carry out routine experimentation to obtain a functional antibody with reduced immunogenicity.

Alterations to the variable region notwithstanding, those skilled in the art will appreciate that a modified antibody or antigen-binding fragment thereof of this disclosure will comprise an antibody (e.g., full-length antibody or antigen-binding fragment thereof) in which at least a fraction of one or more of the constant region domains has been deleted or otherwise altered so as to provide desired biochemical characteristics such as increased tumour localization or reduced serum half-life when compared with an antibody of approximately the same immunogenicity comprising a native or unaltered constant region. In some aspects, the constant region of the modified antibody will comprise a human constant region. Modifications to the constant region compatible with this disclosure comprise additions, deletions or substitutions of one or more amino acids in one or more domains. That is, the modified antibody disclosed herein can comprise alterations or modifications to one or more of the three heavy chain constant domains (CH1, CH2 or CH3) and/or to the light chain constant domain (CL). In some aspects, a modified constant region wherein one or more domains are partially or entirely deleted are contemplated. In some aspects, a modified antibody will comprise domain deleted constructs or variants wherein the entire CH2 domain has been removed (ΔCH2 constructs). In some aspects, the omitted constant region domain can be replaced by a short amino acid spacer (e.g., 10 residues) that provides some of the molecular flexibility typically imparted by the absent constant region.

Besides their configuration, it is known in the art that the constant region mediates several effector functions. For example, antibodies bind to cells via the Fc region, with an Fc receptor site on the antibody Fc region binding to an Fc receptor (FcR) on a cell. There are a number of Fc receptors that are specific for different classes of antibody, including IgG (gamma receptors), IgE (eta receptors), IgA (alpha receptors) and IgM (mu receptors). Binding of antibody to Fc receptors on cell surfaces triggers a number of diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In some aspects, an antibody or an antigen-binding fragment thereof provides for altered effector functions that, in turn, affect the biological profile of the administered antibody or antigen-binding fragment thereof. For example, the deletion or inactivation (through point mutations or other means) of a constant region domain can reduce Fc receptor binding of the circulating modified antibody. In other cases, it can be that constant region modifications, consistent with this disclosure, moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this disclosure can easily be made using well-known biochemical or molecular engineering techniques well within the purview of the skilled artisan.

In some aspects, the antibody or antigen-binding fragment thereof does not have one or more effector functions. For instance, In some aspects, the antibody or antigen-binding fragment thereof has no antibody-dependent cellular cytotoxicity (ADCC) activity and/or no complement-dependent cytotoxicity (CDC) activity. In some aspects, the antibody or antigen-binding fragment thereof does not bind to an Fc receptor and/or complement factors. In some aspects, the antibody or antigen-binding fragment thereof has no effector function.

In some aspects, the antibody or antigen-binding fragment thereof can be engineered to fuse the CH3 domain directly to the hinge region of the respective modified antibodies or fragments thereof. In other constructs a peptide spacer can be inserted between the hinge region and the modified CH2 and/or CH3 domains. For example, compatible constructs can be expressed in which the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. In some aspects, any spacer added to the construct can be relatively non-immunogenic, or even omitted altogether, so as to maintain the desired biochemical qualities of the modified antibodies.

Besides the deletion of whole constant region domains, an antibody or antigen-binding fragment thereof provided herein can be modified by the partial deletion or substitution of a few or even a single amino acid in a constant region. For example, the mutation of a single amino acid in selected areas of the CH2 domain can be enough to substantially reduce Fc binding and thereby increase tumor localization. Similarly, one or more constant region domains that control the effector function (e.g., complement C1Q binding) can be fully or partially deleted. Such partial deletions of the constant regions can improve selected characteristics of the antibody or antigen-binding fragment thereof (e.g., serum half-life) while leaving other desirable functions associated with the subject constant region domain intact. Moreover, the constant regions of the antibody and antigen-binding fragment thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. In some aspects, there may be an addition of one or more amino acids to the constant region to enhance desirable characteristics such as decreasing or increasing effector function or provide for more cytotoxin or carbohydrate attachment. In some aspects, it can be desirable to insert or replicate specific sequences derived from selected constant region domains.

The methods, combinations and kits described herein embrace variants and equivalents that are substantially homologous an antibody or antigen binding fragment of the disclosure (e.g., murine, chimeric, humanized or human antibody, or antigen-binding fragments thereof). These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In some aspects, the antibody or antigen-binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### Definitions

The following definitions pertain, in particular, to the description of topoisomerase I inhibitors above, and may even more particularly pertain to the section entitled “further preferences”.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g., C₅-6) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g., partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g., partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃) and butyl (C₄).

Examples of saturated linear alkyl groups include, but are not limited to, methyl (C₁), ethyl (C₂), n-propyl (C₃) and n-butyl (C₄).

Examples of saturated branched alkyl groups include iso-propyl (C₃), iso-butyl (C₄), sec-butyl (C₄) and tert-butyl (C₄).

C₂₋₄ Alkenyl: The term “C₂₋₄ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂) and butenyl (C₄).

C₂₋₄ alkynyl: The term “C₂₋₄ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₄ cycloalkyl: The term “C₃₋₄ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃) and cyclobutane (C₄); and
unsaturated monocyclic hydrocarbon compounds:
   cyclopropene (C₃) and cyclobutene (C₄).

Connection labels: In the formula

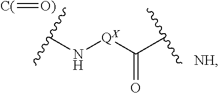

the superscripted labels ^{C(=O)} and ^{NH} indicate the group to which the atoms are bound. For example, the NH group is shown as being bound to a carbonyl (which is not part of the moiety illustrated), and the carbonyl is shown as being bound to a NH group (which is not part of the moiety illustrated).

### Salts

It may be convenient or desirable to prepare, purify, and/or handle a corresponding salt of the active compound/agent, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

### Isomers

Certain compounds/agents of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-HillDictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Enantiomerically enriched form” refers to a sample of a chiral substance whose enantiomeric ratio is greater than 50:50 but less than 100:0.

Note that, except as discussed below for tautomeric forms, specifically excluded from the term “isomers”, as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/enediamine, nitroso/oxime, thioketone/enethiol, N-nitroso/hyroxyazo, and nitro/aci-nitro.

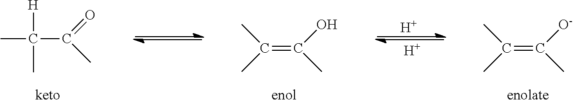

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and 14C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, chlorine and iodine, such as, but not limited to ²H (deuterium, D), ³H (tritium), ¹¹C, ¹³C, ¹⁴C, ¹⁵N, ¹⁸F, ³¹P, ³²P, ³⁵, ³⁶Cl, and ¹²⁵. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### PARP1 Inhibitors

In some aspects, an ADC described herein is administered in combination with an inhibitor of PARP1 ((Poly(ADP-Ribose) Polymerase 1). In some aspects, the inhibitor of PARP1 is AZD5305. The language “inhibit,” “inhibition” or “inhibiting” includes a decrease in the baseline activity of a biological activity or process.

The term “AZD5305” refers to a compound with the chemical name of 5-[4-[(7-ethyl-6-oxo-5H-1,5-naphthyridin-3-yl)methyl]piperazin-1-yl]-N-methyl-pyridine-2-carboxamide and structure shown below:

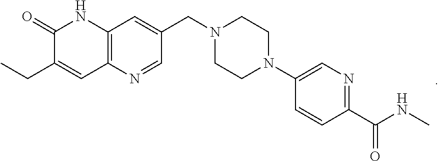 [SMILES: CCc1cc2ncc(CN3CCN(c4ccc(C(=O)NC)nc4)CC3)cc2[nH]c1=O]

Preparation of AZD5305 is disclosed in U.S. Publication. No. US 2021/0040084 A1, the disclosure of which is incorporated by reference in its entirety. In some aspects, a free base of AZD5305, is administered to a subject. In some aspects, a pharmaceutically acceptable salt of AZD5305 is administered to a subject. In some aspects, a crystalline AZD5305 is administered to a subject. In some aspects, crystalline Form A AZD5305 is administered to a subject.

A “pharmaceutical composition” comprising a AZD5305 includes compositions comprising an active ingredient and a pharmaceutically acceptable excipient, carrier or diluent, wherein the active ingredient is AZD5305 or a pharmaceutically acceptable salt thereof.

### ATR Inhibitors

In some aspects, an ADC described herein is administered in combination with an inhibitor of ATR (also known as FRAP-Related Protein 1; FRP1; MEC1; SCKL; SECKL1R). In some aspects, the inhibitor of ATR is AZD6738. The language “inhibit,” “inhibition” or “inhibiting” includes a decrease in the baseline activity of a biological activity or process.

The term “AZD6738” refers to an ATR (also known as FRAP-Related Protein 1; FRP1; MEC1; SCKL; SECKL1R) inhibitor, 4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine; represented by the following formula:

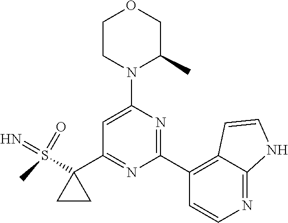 [SMILES: C[C@@H]1COCCN1c1cc(C2([S@](C)(=N)=O)CC2)nc(-c2ccnc3[nH]ccc23)n1]

or a pharmaceutically acceptable salt thereof. Additional examples of ATR inhibitors can be found in U.S. Pat. No. 8,252,802, which is hereby incorporated by reference in its entirety for all purposes.

A “pharmaceutical composition” comprising a AZD6738 includes compositions comprising an active ingredient and a pharmaceutically acceptable excipient, carrier or diluent, wherein the active ingredient is AZD6738 or a pharmaceutically acceptable salt thereof.

### Combinations

The present disclosure further provides combinations of an ADC and a PARP1 inhibitor for the treatment of cancer. Or an ADC and an ATR inhibitor for the treatment of cancer.

In some aspects, the disclosure provides a combination of: A) an antibody-drug conjugate (ADC) comprising: i. an antibody or antigen binding fragment thereof which binds to a B7-H4 polypeptide, ii. a cleavable linker; and iii. a cytotoxic agent; and B) a PARP1 (Poly(ADP-Ribose) Polymerase 1) inhibitor for treating cancer in a human subject in need thereof. In some aspects, the disclosure provides a combination of: A) an antibody-drug conjugate (ADC) comprising: i. an antibody or antigen binding fragment thereof which binds to a B7-H4 polypeptide, ii. a cleavable linker; and iii. a cytotoxic agent; and B) an ATR inhibitor for treating cancer in a human subject in need thereof.

In some aspects, the disclosure provides a combination of: A) an ADC comprising: i. an antibody or antigen binding fragment thereof which binds to a B7-H4 polypeptide, ii. a cleavable linker; and iii. a cytotoxic agent; and B) AZD5305 or a pharmaceutically acceptable salt thereof for treating cancer in a human subject in need thereof.

In some aspects, the disclosure provides a combination of: A) an ADC comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof; c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof, d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD5305 or a pharmaceutically acceptable salt thereof for treating cancer in a human subject in need thereof.

In some aspects, the disclosure provides a combination of: A) an ADC comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof, c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof; d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD6738 or a pharmaceutically acceptable salt thereof for treating cancer in a human subject in need thereof.

In some aspects of the combinations provided herein, the cancer to be treated is a cancer as described herein. In some aspects, the cancer is selected from ovarian cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is ovarian cancer. In some aspects of the method for treating breast cancer, breast cancer is a hormone receptor-positive (HR+) breast cancer, human epidermal growth factor receptor 2 positive (HER2⁺) breast cancer, or a triple negative breast cancer (TNBC). In some aspects, the breast cancer is TNBC.

In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L. In some aspects, the mutated HRD gene is selected from BRCA1, BRCA2, and ATM. In some aspects, the mutated HRD gene is BRCA1. In some aspects, the mutated HRD gene is BRCA2. In some aspects, the mutated HRD gene is ATM.

In some aspects of the combinations provided herein, the ADC can have any antibody or antigen binding fragment thereof as described herein. In some aspects of the combinations provided herein, the ADC can have any linker as described herein. In some aspects of the combinations provided herein, the ADC can have any cytotoxic agent as described herein.

In some aspects of the combinations provided herein, the ADC comprises an antibody or antigen binding fragment thereof as described herein conjugated with SG3932, SG4010, SG4057, SG4052 or combinations thereof. In some aspects of the combinations provided herein, the ADC comprises an antibody or antigen binding fragment thereof as described herein conjugated with SG3932. The linker-cytotoxic agent molecules SG3932, SG4010, SG4057 and SG4052 are described elsewhere herein.

### Kits

In one aspect, there is provided a kit comprising an ADC as described herein and a PARP1 inhibitor as described herein. In one aspect, there is provided a kit comprising an ADC as described herein and an ATR inhibitor as described herein. There is further embraced use of said kit in the methods of the present disclosure.

In some aspects, the present disclosure provides a kit comprising: A) an antibody-drug conjugate (ADC) comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof, c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof; d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof, or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD5305 or a pharmaceutically acceptable salt thereof.

In some aspects, the present disclosure provides a kit comprising: A) an antibody-drug conjugate (ADC) comprising: i. an antibody or antigen binding fragment thereof which binds to a B7-H4 polypeptide comprising: a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof; b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof, c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof; d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof, or e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; ii. a cleavable linker; and iii. a cytotoxic agent; and B) AZD6738 or a pharmaceutically acceptable salt thereof.

In some aspects of the kits provided herein, the ADC can have any antibody or antigen binding fragment thereof as described herein. In some aspects of the combinations provided herein, the ADC can have any linker as described herein. In some aspects of the combinations provided herein, the ADC can have any cytotoxic agent as described herein.

In some aspects of the kits provided herein, the ADC comprises an antibody or antigen binding fragment thereof as described herein conjugated with SG3932, SG4010, SG4057, SG4052 or combinations thereof. In some aspects of the combinations provided herein, the ADC comprises an antibody or antigen binding fragment thereof as described herein conjugated with SG3932. The linker-cytotoxic agent molecules SG3932, SG4010, SG4057 and SG4052 are described elsewhere herein.

In some aspects, a kit comprises an isolated (e.g., purified) antigen or antibody binding fragment as described herein. In some aspects, a kit comprises an isolated (e.g., purified) ADC as described herein. In some aspects, the kit comprises one or more container. The kit may provide the antigen or antibody binding fragment and the linker and/or cytotoxic agent individually (e.g., the agent is not conjugated to the antigen or antibody binding fragment, but is in a form suitable for conjugation thereto); optionally wherein the kit is further provided with instructions and/or reagents for conjugating the agent to the antigen or antibody binding fragment. In some aspects, the kit comprises all of the components necessary and/or sufficient to administer the combination of ADC and PARP1 inhibitor to a subject. In some aspects, the kit comprises all of the instructions necessary and/or sufficient to administer the combination of ADC and PARP1 inhibitor to a subject.

### EXAMPLES

The combination therapy disclosed herein will now be further explained by reference to the following non-limiting examples.

### Example 1—Cytotoxicity Assay with DLD-1-BRCA WT Cells Engineered to Express B7-H4

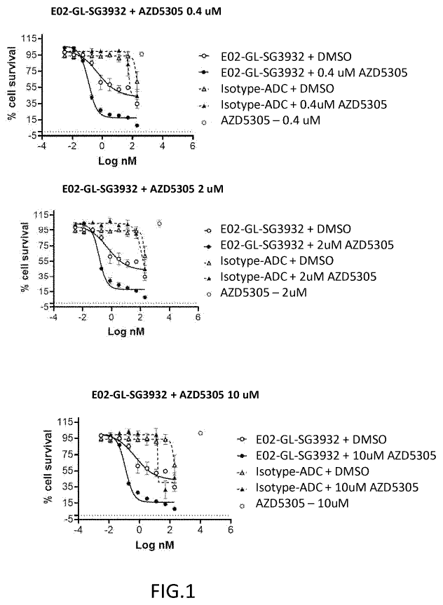

To determine the efficacy of combined B7-H4-TOPli ADC (E02-GL-SG3932) and PARP1 selective inhibitor (AZD5305) therapy of killing DLD-1-BRCA wild-type (WT) cells expressing B7-H4, a cytotoxicity assay was performed. DLD-1-BRCA WT cells (Horizon Discovery), engineered to express B7-H4, were plated in 96-well plates at a seeding density of 1,000 cells/well in RPMI medium containing 10% heat-inactivated FBS. On the following day, cells were treated with increasing concentrations of E02-GL-SG3932, an isotype-matched control ADC (0.003052-200 nM), or AZD5305 (0.4-10 μM), individually or in combination. Cells were incubated for 7 days post treatment and cell viability was measured using the CellTiter-Glo assay (Promega), which quantifies cell viability based on the amount of adenosine triphosphate (ATP) present. Results show that at each of the increasing concentrations, the combined E02-GL-SG3932 and AZD5305 treatment killed more WT cells expressing B7-H4, at a faster rate, than did the other treatments: E02-GL-SG3932 individually, AZD5305 individually, an isotype-matched control ADC, and isotype-matched control ADC and AZD5305 (FIG. 1).

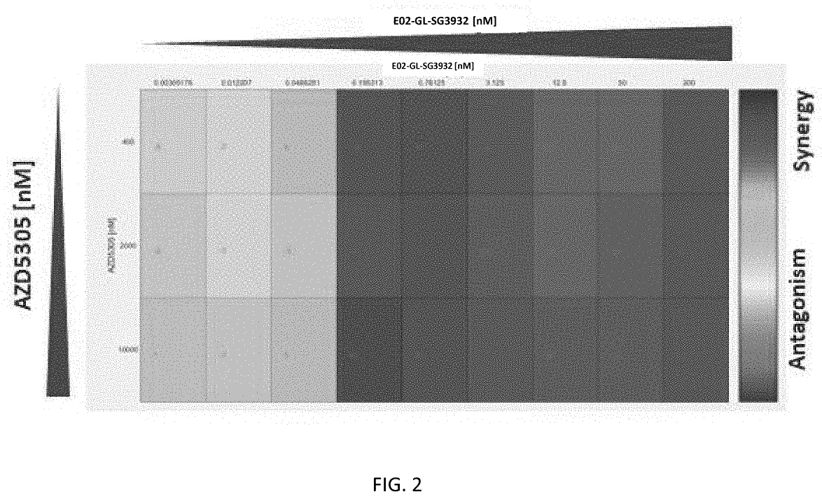

The Bliss synergy model was used to estimate the two-drug combination effect and pharmacologic synergy (Bliss score>0) or antagonistic (Bliss score<0) response of AZD5305 in combination with the E02-GL-SG3932 ADC, determined using the Combenefit software (Cancer Research UK Cambridge Institute). Veroli et al Bioinformatics. 2016 Sep. 15; 32(18): 2866-2868. As shown in FIG. 2, increasing dosages in the combination treatment resulted in Bliss energy scores of larger than 10 (blue), which indicates treatment synergy between E02-GL-SG3932 and AZD5305 at increased dosages.

### Example 2—Cytotoxicity Assay with DLD-1-XMAN-BRCA2-/-Cells Engineered to Express B7-H4

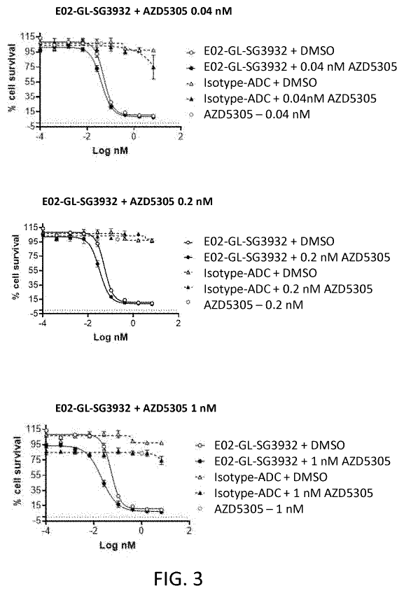

DLD-1-XMAN-BRCA2-/-cells, engineered to express B7-H4, were used to assess the efficacy of the combination E02-GL-SG3932 and AZD5305 treatment in killing target cells. The DLD-1-XMAN-BRCA2-/-cells lack the BRCA2 gene, which provides instructions for making a protein that acts as a tumor suppressor. DLD-1-XMAN-BRCA2-/-cells (Horizon Discovery), engineered to express B7-H4, were plated in 96-well plates at a seeding density of 1,000 cells/well in RPMI medium containing 10% heat-inactivated FBS. On the following day, cells were treated with increasing concentrations of E02-GL-SG3932, an isotype-matched control ADC (0.0001-6.7 nM), or AZD5305 (0.04-1 nM), individually or in combination. Cells were incubated for 7 days post treatment and cell viability was measured using the CellTiter-Glo assay (Promega). As seen in FIG. 3, the 1 nM combination treatment killed more DLD-1-XMAN-BRCA2-/-cells expressing B7-H4 at a faster rate, than did the other treatments of E02-GL-SG3932 individually, AZD5305 individually, an isotype-matched control ADC, and isotype-matched control ADC and AZD5305.

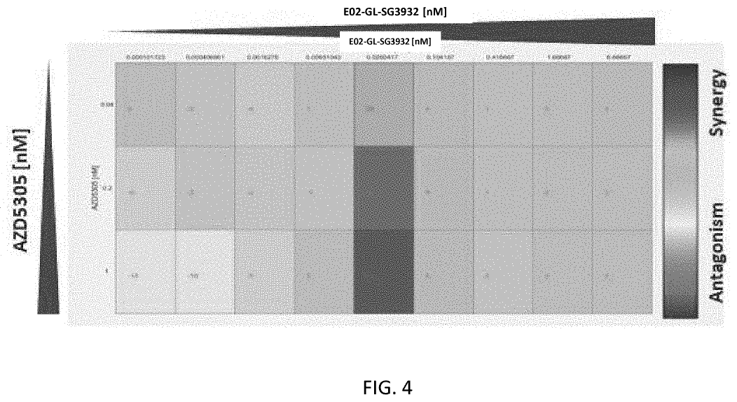

The Bliss synergy model was used to estimate the two-drug combination effect and pharmacologic synergy (Bliss score>0) or antagonistic (Bliss score<0) response of AZD5305 in combination with the E02-GL-SG3932 ADC, determined using the Combenefit software (Cancer Research UK Cambridge Institute). As shown in FIG. 4, dosages of E02-GL-SG3932 (0.02 nM) in combination with AZD5305 (0.04 nM, 0.2 nM and lnM) resulted in Bliss energy scores of larger than 10 (blue), which indicates treatment synergy between E02-GL-SG3932 and AZD5305.

### Example 3—Cytotoxicity Assay with MX-ICells Engineered to Express B7-H4

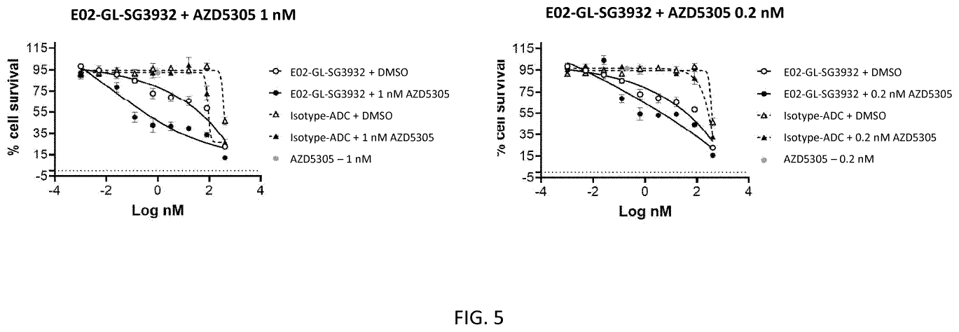

MX-1 cells established as in vitro culture from MX-1 tumor xenograft models of breast carcinoma tissue were used to assess the efficacy of the combination E02-GL-SG3932 and AZD5305 treatment in killing target cells. MX-1 cells were plated in 96-well plates at a seeding density of 1,500 cells/well in DMEM medium containing 10% heat-inactivated FBS. The following day, cells were treated with increasing concentrations of E02-GL-SG3932, an isotype-matched control ADC (0.001-400 nM), or AZD5305 (0.2 or lnM), individually or in combination. Cells were incubated for 7 days post treatment and cell viability was measured using the CellTiter-Glo assay (Promega). FIG. 5 shows the ability of the combination E02-GL-SG3932 and AZD5305 treatments at 0.2 nM and 1 nM concentrations to kill MX-1 cells at greater amounts than the other treatments of E02-GL-SG3932 individually, AZD5305 individually, an isotype-matched control ADC, and isotype-matched control ADC and AZD5305.

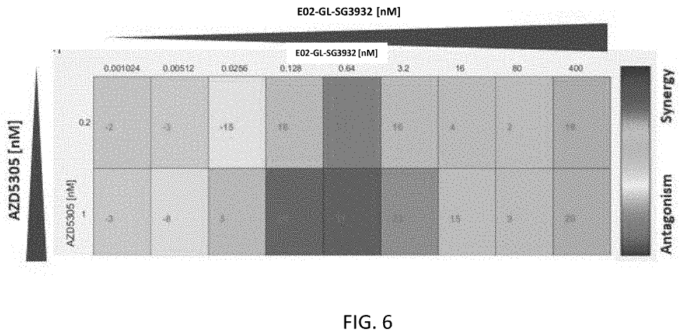

The Bliss synergy model was used to estimate the two-drug combination effect and pharmacologic synergy (Bliss score>0) or antagonistic (Bliss score<0) response of AZD5305 in combination with the E02-GL-SG3932 ADC, determined using the Combenefit software (Cancer Research UK Cambridge Institute). FIG. 6 shows that dosages of E02-GL-SG3932 (0.128-3.2 nM and 400 nM) in combination with AZD5305 (lnM) resulted in Bliss energy scores of ≤16 and larger than 10 (blue), which indicates treatment synergy between E02-GL-SG3932 and AZD5305 at these combinations.

### Example 4—MDA-MB-468 Breast Cancer Xenograft Models

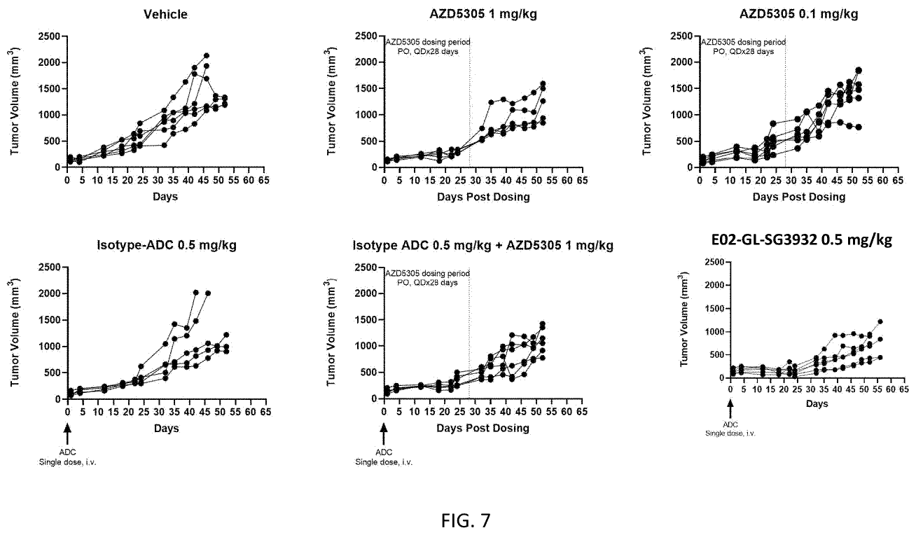

In vivo experiments were performed to measure the anti-tumor efficacy of E02-GL-SG3932 and AZD5305 treatment in mice bearing xenografts of human estrogen-independent MDA-MB-468 breast cancers. MDA-MB-468 cells were injected 5×10⁶ per mouse into the mammary fat pad of female CB-17 SCID mice. Dosing of the Isotype-ADC, E02-GL-SG3932, AZD5305, or combinations were initiated when tumors reached an average of 150-200 mm³. The isotype control ADC and E02-GL-SG3932 were dosed as a single IV tail vein injection on day 1 only, and for combination studies, this injection was followed by once daily oral dosing of AZD5305 for 28 days. Tumor and body weight measurements were recorded twice weekly for the duration of the study. Tumors measured over time in mice treated with the vehicle (control), AZD5305 individually (0.1 mg/kg and 1 mg/kg), isotype-matched control ADC (0.5 mg/kg), isotype-matched control ADC and AZD5305 (1 mg/kg), and E02-GL-SG3932 individually (0.1 mg/kg and 1 mg/kg) increased in or maintained size, which indicates these treatment methods exhibit little to no anti-tumor activity in vivo (FIG. 7).

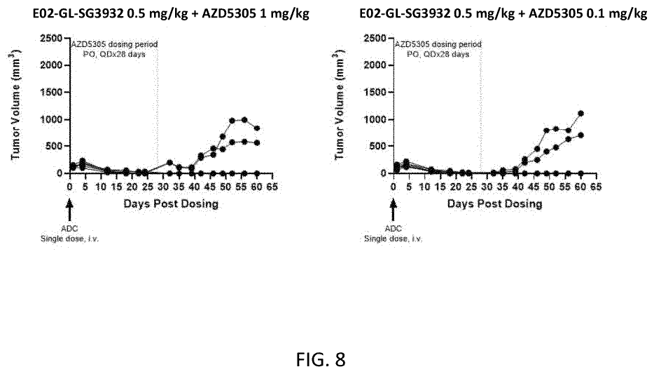

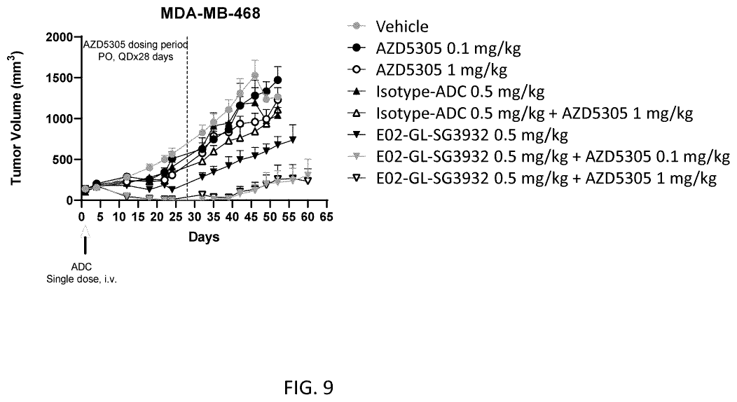

E02-GL-SG3932 (0.5 mg/kg) and AZD5305 (0.1 mg/kg), and E02-GL-SG3932 (0.5 mg/kg) and AZD5305 (1 mg/kg) treatments exhibited high anti-tumor activity. As shown in FIG. 8, combination treatments of E02-GL-SG3932 and AZD5305 were effective in inhibiting the growth of approximately 67% of tumors in the mice. Further comparative analysis of the efficacy of the various treatment combinations in mice is shown in FIG. 9. The mean tumor volumes in mice treated with E02-GL-SG3932 (0.5 mg/kg) and AZD5305 (0.1 mg/kg), and E02-GL-SG3932 (0.5 mg/kg) and AZD5305 (1 mg/kg) were significantly smaller than that of the mean tumor volumes of the other treatment methods.

### Example 5—MDA-MB-468 Breast Cancer Xenograft Models

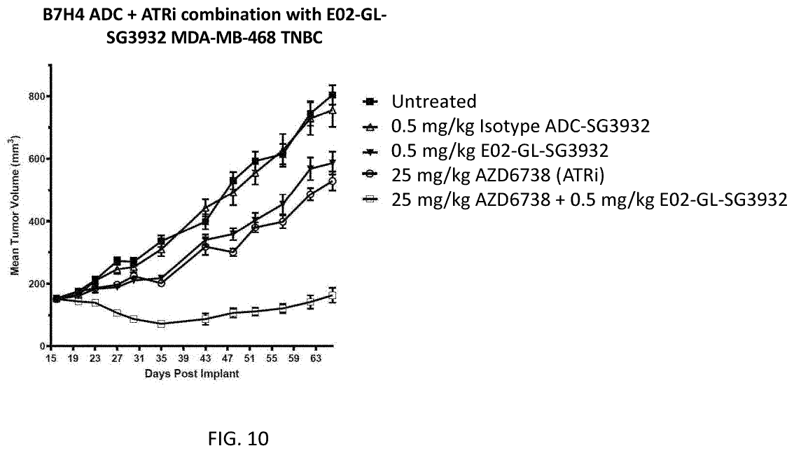

In vivo experiments were performed to measure the anti-tumor efficacy of E02-GL-SG3932 and AZD6738 treatment in mice bearing xenografts of human estrogen-independent MDA-MB-468 breast cancers. MDA-MB-468 cells were injected 5×10⁶ per mouse into the mammary fat pad of female CB-17 SCID mice. The isotype control ADC and E02-GL-SG3932 were dosed as a single IV tail vein injection on day 1 only, and for combination studies, this injection was followed by twice daily (BID) oral dosing of AZD6738 for 14 days. Tumor and body weight measurements were recorded twice weekly for the duration of the study. Tumors measured over time in mice treated with the untreated (control), AZD6738 individually (25 mg/kg), isotype-matched control ADC (0.5 mg/kg), E02-GL-SG3932 individually (0.5 mg/kg), and E02-GL-SG3932 (0.5 mg/kg) and AZD6738 (25 mg/kg) are shown in FIG. 10.

As shown in FIG. 10, combination treatments of E02-GL-SG3932 and AZD6738 were effective in further inhibiting the growth of tumors in the mice. The mean tumor volumes in mice treated with E02-GL-SG3932 (0.5 mg/kg) and AZD6738 (25 mg/kg) were significantly smaller than that of the mean tumor volumes of the other treatment methods.

### Example 6—Preclinical Evaluation of a B7-H4-Directed Antibody-Drug Conjugate in Combination with a PARP1-Selective Inhibitor

This study evaluated the activity of E02-GL-SG3932, a B7-H4-directed antibody-drug conjugate (ADC) bearing a novel topoisomerase I inhibitor (TOPli) payload in combination with the poly-ADP ribose polymerase 1 (PARP1)-selective inhibitor AZD5305, in preclinical models. A comprehensive analysis of B7-H4 prevalence and an assessment of intratumoral heterogeneity in human tumors was conducted. Addition of the next-generation PARP1-selective inhibitor AZD5305 to E02-GL-SG3932 treatment sensitized low B7-H4-expressing PDX tumors, including homologous-proficient models that represent that represent acquired or intrinsic mechanisms of resistance to PARP inhibition. These findings support the implementation of the combination of an anti-B7H4 ADC and a PARP inhibitor even in subjects that have already been treated with PARP inhibitors and subjects that are resistant to treatment with PARP inhibitors.

### Materials and Methods

### Antibodies

Antibodies targeting human B7-H4 were generated in VelocImmune V2 mice by alternating immunization with SKBR3 cells and mouse B7-H4 extracellular domain chemically conjugated to keyhole limpet hemocyanin. Lead antibodies were selected based on specificity and cross-reactivity to human and cynomolgus monkey B7-H4, and antibody frameworks were mutated to the closest human germlines.

### Cell Lines

HT29 and MDA-MB-468 cells were obtained from Deutsche Sammlung von Mikroorganismen und Zellkulturen, GmbH (Braunschweig, Germany) and American Type Culture Collection (Rockville, MD, USA), respectively, and were cultured in RPMI 1640 medium containing 10% heat-inactivated fetal bovine serum (FBS) (Thermo Fisher Scientific, Waltham, MA, USA). MX-1 cells were obtained from the National Cancer Institute (Bethesda, MD, USA) and cultured in Dulbecco modified essential/F12 medium containing 10% FBS and 2 mM L-glutamine (Thermo Fisher Scientific). The HT29 clonal cell line stably expressing human B7-H4 (HT29-huB7-H4) was generated by using lentivirus expression vectors prepared with the pPACK H1 HIV Lentivector Packaging Kit (System Biosciences, Palo Alto, CA, USA), followed by transduction, geneticin selection, and isolation with fluorescence-activated cell sorting through direct staining with an anti-B7-H4-PE antibody (US Biological, Salem, MA, USA). All cell lines were cultured in a humidified atmosphere of 5% CO₂ at a temperature of 37° C., authenticated by short tandem repeat DNA profiling (IDEXX BioResearch Laboratories, Columbia, MO, USA), and found to be negative for Mycoplasma by polymerase chain reaction and the MycoSEQ Mycoplasma detection assay kit (Thermo Fisher Scientific).

### Immunohistochemistry

Immunohistochemistry (IHC) detection of B7-H4 was performed on 4-μm formalin-fixed, paraffin-embedded tumor or tissue sections with an automated Leica Bond RX IHC staining platform (Leica Biosystems, Nussloch, Germany). Human normal and tumor tissue sections were stained with anti-human B7-H4 mouse immunoglobulin G1 (IgG1) (clone ZYOEYT-D11; AstraZeneca, Cambridge, UK), and tumor tissue sections derived from patient-derived xenografts (PDX) were stained with anti-human B7-H4 rabbit IgG1. Both antibodies were thoroughly validated for use in IHC with a range of known B7-H4-expressing cell lines, B7-H4 transfected cell lines (HT29-huB7H4), and human breast tumor tissue (MX-1 and MDA-MB-468) to ensure specificity and sensitivity (Table 1). B7-H4 negative HT29 cells were used as a negative control. Formalin-fixed, paraffin-embedded cell pellets were prepared from the same cells for orthogonal analysis of B7-H4 expression by IHC. After being cover slipped and mounted, slides were scanned with a Leica Aperio Scanscope AT2 slide scanner and then reviewed and scored by a pathologist. Antibody binding capacity, a measure of B7-H4 receptor density, was measured by quantitative flow cytometry with the Quantum Simply Cellular kit from Bangs Laboratories (Fisher, IN, USA).

TABLE 1
B7H4 Expression in Different Cell Types

| Cell line | B7-H4 receptor density | B7-H4 IHC expression |
|---|---|---|
| MX-1 | 903,249 | +++ |
| HT29-huB7H4 | 538,257 | +++ |
| OvCAR4 | 357,257 | +++ |
| OAW28 | 353,432 | ++ |
| SKBR3 | 292,103 | ++ |
| MDA-MB-468 | 170,669 | +/++ |
| HCC1569 | 46,557 | +/++ |
| HT29 | 101 | − |

### Preparation of ADCs

The various TOPli warheads were covalently bound to native cysteines in the antibody through a val-ala (VA) or Gly-Gly-Phe-Gly (GGFG) peptide linker with or without a PEG₈ spacer, resulting in four distinct ADCs with an approximate drug-to-antibody ratio (DAR) of 8:SG3932, SG4057, SG4010 and SG4052. Preparation of these ADCs is also described in WO2022053650, which is hereby incorporated by reference in its entirety. A non-targeting isotype control ADC (Iso-ADC) was synthesized in the same manner by using an isotype-matched antibody with a comparable DAR. The E02-GL-SG3932 antibody drug conjugate is E02-GL antibody conjugated to the SG3932 warhead.

A 100 mM solution of TCEP [tris(2-carboxyethyl)phosphine] in PBS was added (12.5 molar equivalent/antibody, 0.3 mmol) to E02-GL antibody (3.6 g, 24.0 micromoles) in reduction buffer containing PBS and 1 mM ethylenediaminetetraacetic acid and a final antibody concentration of 10 mg/mL. The reduction mixture was heated at 37° C. for 2 hours (or until full reduction was observed by ultrahigh-performance liquid chromatography [UPLC]) in an orbital shaker with gentle (60 rpm) shaking. Reduced antibody was removed from incubation and allowed to cool to room temperature. To this mixture, SG3932, SG4057, SG4010, or SG4052 linker-payload was added as a dimethyl sulfoxide (DMSO) solution (11-14 molar equivalent/antibody, 26.4-33.6 micromoles) for a 10% (v/v) final DMSO concentration. The solution was shaken for 1 hour at 25° C. and then the conjugation was quenched with N-acetyl cysteine (5×excess compared to payload as a 100 mM stock solution). Excess free drug was removed via tangential flow filtration unit, using mPES, MidiKros 30-kDa fiber filter with a 375-cm² surface area, into buffer containing 30 mM histidine, 30 mM arginine, pH 6.8. Extent of free drug removal was monitored by UPLC-RP with neat conjugate. After complete removal of free drug (11-16 DV), antibody-drug conjugate (ADC) was formulated into 20 mM histidine, 240 mM sucrose, pH 6.0 (5 DV), and the resultant ADC was filtered with a 0.22-μm filter under a sterile atmosphere. Drug-to-antibody ratio (DAR) was calculated by UPLC-RP with reduced conjugate, and monomeric purity was measured by UPLC-size exclusion chromatography (SEC) with neat conjugate.

### In Vivo Efficacy Studies

All animal experiments were conducted in a facility accredited by the Association for Assessment and Accreditation of Laboratory Animal Care under the guidelines of AstraZeneca's Institutional Animal Care and Use Committee and appropriate animal research approvals. Cell line xenograft models were established through subcutaneous implantation of cells in 50% Matrigel (Corning, Corning, NY, USA) in either the right mammary fat pad (MX-1, MDA-MB-468) or left flank (HT29, HT29-huB7-H4) of female CB-17 mice (Envigo, Frederick, MD, USA). When tumor volume reached 150-200 mm³, mice were randomized and given a single intravenous (IV) injection of vehicle control (20 mM histidine, pH 6; 240 mM sucrose; 0.02% PS-80) or ADCs. Body weight and tumor measurements were determined once or twice weekly, and tumor volume was calculated with the formula

$$Tumor volume ( {mm}^{3} ) = \frac{[ length ( mm ) \times {width ( mm )}^{2} ]}{2} .$$

PDX models of human TNBC were established through subcutaneous implantation of tumor fragments into the flank of athymic nude mice (Envigo, Riom, France) and tested at Xentech (Evry, France). Animals were matched by tumor volume, and vehicle control or ADCs were then administered as a single IV injection. When used, the PARP1-selective inhibitor AZD5305 (AstraZeneca) was formulated in water/HCl pH 3.5-4 and administered by oral gavage at a final dose volume of 10 mL/kg once daily for 28 days. Antitumor activity was determined on the last day, at which time all mice in the untreated control group remained in the study. If the end tumor volume (ETV) in the treatment group was less than the initial tumor volume (ITV), the antitumor response from baseline was calculated from the group means by using the formula

$$[ \frac{( ETV - ITV )}{ITV} ] \times 100 .$$

Otherwise, the antitumor response was expressed as percent change in tumor volume in the treatment arm relative to that in the untreated control arm, calculated by the formula

$$100 - \{ 1 - [ \frac{( ETV - ITV ) treated}{( ETV - ITV ) untreated} ] \times 100 \} .$$

For pharmacodynamic and pharmacokinetic studies, mice bearing either HT29 or HT29-huB7-H4 tumors (250-300 mm³) were administered a single IV injection of vehicle control or ADCs. Plasma and tumor samples were collected at indicated time points, when one portion of the tumor was fixed in formalin and embedded into paraffin blocks for IHC analysis, and the remaining portion was snap-frozen for analysis of total monoclonal antibody, ADC, and free warhead by liquid chromatography-mass spectrometry.

### Statistical Analysis

Data are presented as mean±standard deviation or mean±standard error of the mean. To evaluate associations with response versus nonresponse in the PDX models, the Wilcoxon test for group comparison was performed in the R statistical programming environment (version 4.1). P<0.05 was considered statistically significant.

### B7-H4 Expression and Prevalence in Tumor Tissues

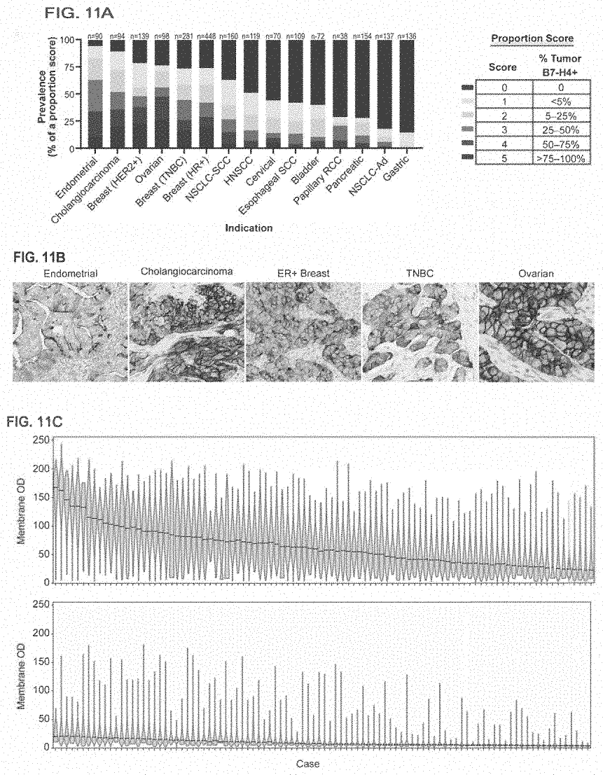

B7-H4 was expressed in several tumor types and was most prevalent in endometrial carcinoma (94%), cholangiocarcinoma (89%), breast cancer (HER2⁺78%, TNBC 74%, ER⁺ 74%), and ovarian carcinoma (77%) (FIGS. 11A, 11B). In contrast, B7-H4 was detected in a limited number of normal human tissues, such as fallopian tube, lung, breast, and prostate, and was generally found in <10% of the total cells in the sample, restricted toward ductal or tubular epithelium, and primarily located on the apical luminal membrane (Table 2).

To understand B7-H4 intratumoral heterogeneity within individual tumors, a key factor influencing the activity of ADCs, a separate collection of histologically annotated samples of TNBC (n=196) were stained and digitally analyzed with deep-learning-based image analysis algorithms to detect and segment the individual tumor epithelial cells and quantify the level of B7-H4 expression on each cell's membrane. This approach allowed for the study of the distribution of B7-H4 expression on a per-cell basis, showing a range of heterogeneous B7-H4 expression within each patient sample across the cohort (FIG. 11C). B7-H4 expression and heterogeneity of expression were weakly correlated (r=−0.84, P<0.01).

TABLE 2
B7-H4 expression in human normal tissue

| | No. of | | | | |
|---|---|---|---|---|---|
| | donors with | Relative | | | |
| | B7-H4 IHC | proportion | | | |
| | staining/tot | of tissue with | | Cellular | |
| Tissue^{a} | al no. | B7-H4⁺ staining | Intensity | localization | Details |
| Breast | 6/6 | <10% | +/+++ | Luminal m | Ductal epithelial cells |
| Epididymis | 3/3 | <5% | ++ | Luminal m | Luminal membrane in ducts |
| Fallopian | 3/3 | <30% | ++/+++ | Luminal m | Ductal epithelial staining |
| tube | | | | | |
| Kidney | 11/11 | <5% | ++/+++ | Luminal m > c | Luminal membrane and some |
| | | | | | cytoplasmic staining in |
| | | | | | occasional tubules |
| Liver | 2/3 | 1% | +/++ | Luminal m | Luminal membrane of |
| | | | | | occasional small bile ducts |
| Lung- | 7/7 | <20% | ++/+++ | Luminal m > c | Basal cells in bronchial |
| bronchus | | | | | epithelium and occasional |
| | | | | | ductal epithelium from |
| | | | | | bronchial glands |
| Esophagus | 1/3 | 1% | 1 | Luminal m | Basolateral membrane of |
| | | | | | occasional epithelial cells |
| | | | | | in submucosal glands |
| Ovary | 3/6 | 1% | +/++ | c > m | Individual mature oocyte |
| | | | | | showing granulosa cells |
| Pancreas | 8/8 | <5% | +/+++ | Luminal m | Luminal membrane and |
| | | | | | cytoplasm of centroacinar |
| | | | | | cells and luminal staining |
| | | | | | of intercalated ducts |
| Pituitary | 3/3 | <5% | ++/+++ | m > c | Occasional localised cells |
| | | | | | within Pars Intermedia cells |
| | | | | | showing staining of colloid |
| | | | | | in follicles and Rathke's |
| | | | | | Cleft epithelial cells |
| Prostate | 3/3 | <10% | ++/+++ | m > c | Basal epithelial cells in |
| | | | | | some ducts |
| Seminal | 3/3 | <10% | ++/+++ | Luminal m | Several ductular epithelial |
| gland | | | | | cells |
| Skin | 15/16 | <5% | ++/+++ | Luminal m | Membrane of occasional |
| | | | | | sweat gland duct epithelium, |
| | | | | | membrane of hair follicle |
| | | | | | epithelium |
| Ureter | 3/3 | <5% | +/++ | m > c | Cytoplasmic staining in some |
| | | | | | basal urothelial cells |
| Urinary | 8/8 | <10% | ++/+++ | Luminal m > c | Apical aspect of urothelial |
| bladder | | | | | cells |

### Combination of E02-GL-SG3932 with Next-Generation PARP1-Selective Inhibitor AZD5305

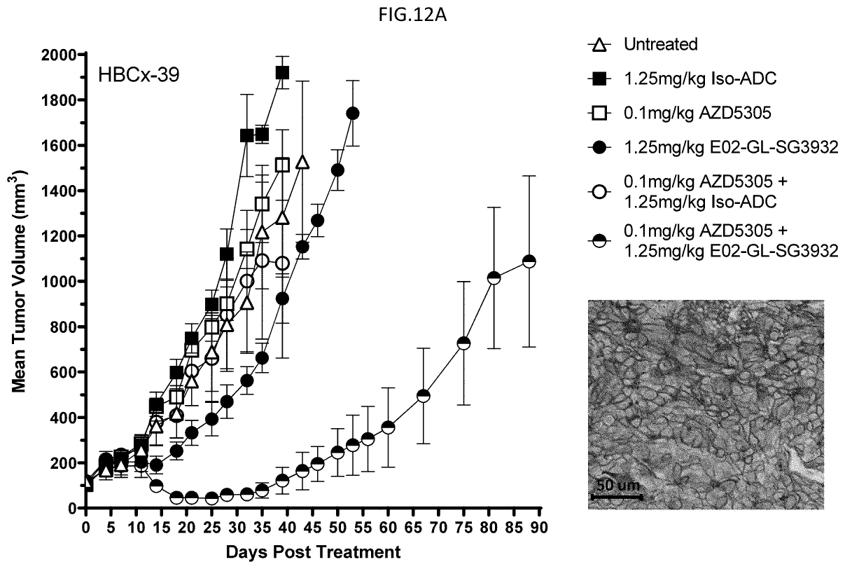

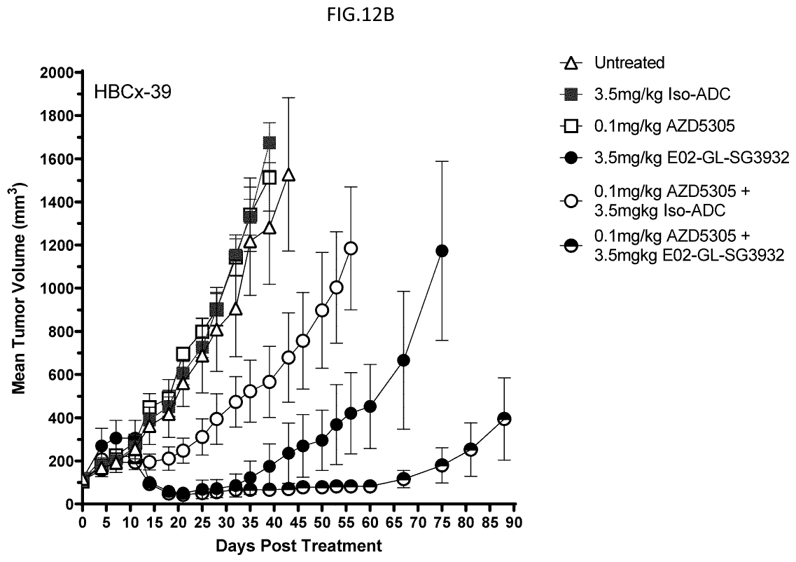

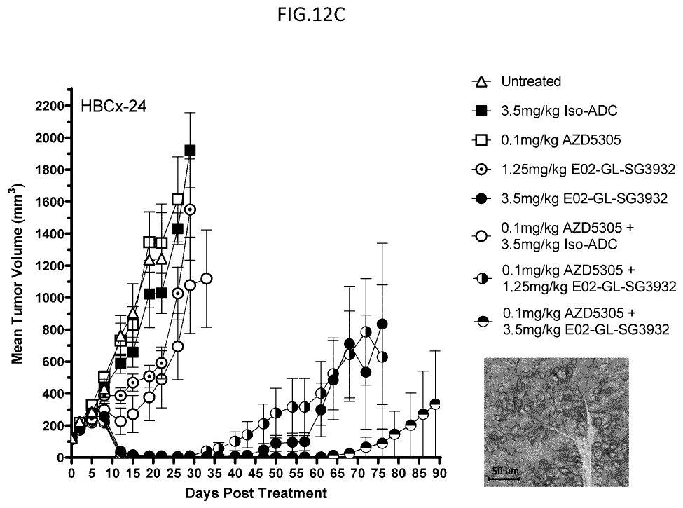

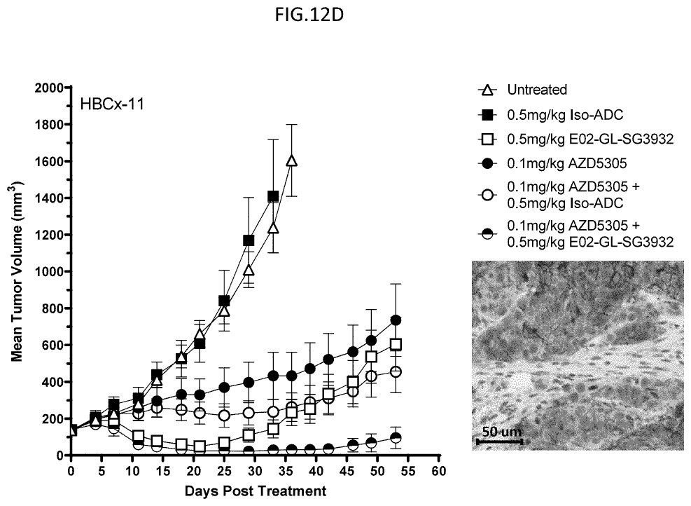

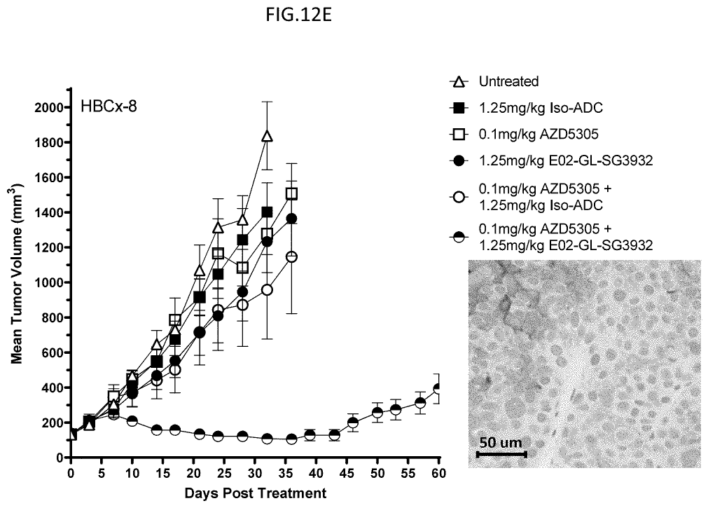

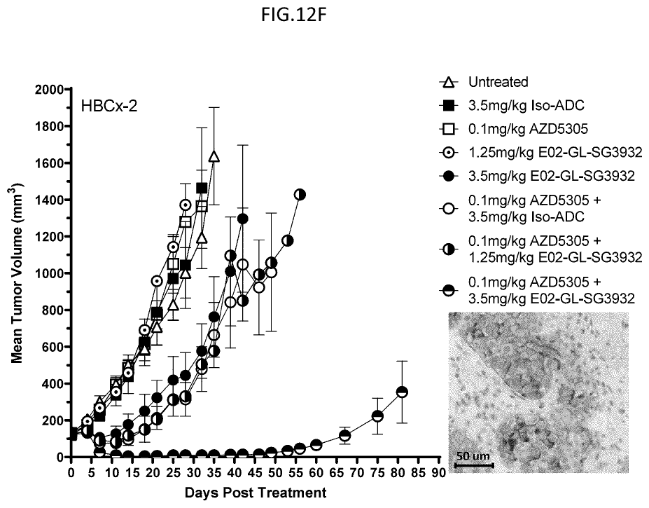

PARP1 limits the cytotoxicity of TOPli by enhancing the excision and repair of TOP1 cleavage complexes. Therefore, E02-GL-SG3932 was tested in combination with a next-generation PARP1-selective inhibitor, AZD5305, in a collection of HRP PDX models, including BRCA WT tumors and models representing post-PARP resistance mechanisms. In the high B7-H4-expressing, BRCA WT HBCx-39 model, AZD5305 sensitized the tumor to a single, suboptimal dose of 1.25 mg/kg E02-GL-SG3932, resulting in tumor regression in five of five mice, in contrast to 24.5% TGI observed in the E02-GL-SG3932 monotherapy arm (FIG. 12A). Greater activity of combination treatment was also observed with a higher dose (3.5 mg/kg) of E02-GL-SG3932, extending the duration of response over that of E02-GL-SG3932 monotherapy (FIG. 12B). Similar results were obtained in other high B7-H4-expressing models, including the BRCA1-mutant HBCx-24 model (FIG. 12C), where PARPi resistance is probably driven through BRCA1 promotor methylation, and the HBCx-11 model (FIG. 12D), which is BRCA1 hypomorphic and resistant to PARPi. The combination benefit observed in HBCx-39 and HBCx-11 is intriguing, as these models not only represent a BRCA WT (HBCx-39) or PARPi-resistant setting (HBCx-11) but are also SLFN11 negative. Because SLFN11 loss is a proposed resistance biomarker for both TOPli and PARPi, the robust combination activity observed in these models is unexpected. It was also surprising that combination benefit extended to HBCx-8, which has very low B7-H4 expression and is a BRCA1-mutant HRP model resistant to PARPi (FIG. 12E). In this model, a single dose of 1.25 mg/kg E02-GL-SG3932 provided modest antitumor activity (32.9% TGI), whereas combination with AZD5305 provided tumor regression in five of five mice. Similar combination benefit was observed in a low B7-H4-expressing BRCA WT model, HBCx-2 (FIG. 12F), as improved activity was observed with increasing doses of the ADC.

Pharmacologic inhibition of the DNA damage response pathway is an attractive approach to maximize the response to TOPli-ADCs and provide greater antitumor activity. This study evaluated the combination of E02-GL-SG3932 and AZD5305, a potent PARP1-selective inhibitor demonstrating reduced hematological toxicity in preclinical models in comparison with non-selective PARPi. The combination of E02-GL-SG3932 and AZD5305 provided greater antitumor activity than monotherapy, resulting in greater tumor regression or improved duration of response even in models with low B7-H4 expression. This combination benefit was observed with low, suboptimal doses of E02-GL-SG3932 as well as in HRP BRCA1 WT or BRCA1-mutant models representing mechanisms of PARPi resistance. These findings suggest that this combination may be effective in a PARPi-resistant setting and that maximum ADC activity need not be achieved to capitalize on this mechanism.

### SEQUENCES

| Identifier | Sequence |
|---|---|
| ZY0EPQ-E02; CHDR1 | SEQ ID NO: 1: GYYWN |
| ZY0EPQ-E02; CHDR2 | SEQ ID NO: 2: EINHSGSTNYNPSLKS |
| ZY0EPQ-E02; CHDR3 | SEQ ID NO: 3: NLYNWNLDS |
| ZY0EPQ-E02; CLDR1 | SEQ ID NO: 4: RASQGIRNDLG |
| ZY0EPQ-E02; CLDR2 | SEQ ID NO: 5: VASSLQS |
| ZY0EPQ-E02; CLDR3 | SEQ ID NO: 6: LQHNSYPRT |
| ZY0EQD-E02; CHDR1 | SEQ ID NO: 7: GYYWN |
| ZY0EQD-E02; CHDR2 | SEQ ID NO: 8: EINHSGSTSYNPSLKS |
| ZY0EQD-E02; CHDR3 | SEQ ID NO: 9: VLYNWNVDS |
| ZY0EQD-E02; CLDR1 | SEQ ID NO: 10: RASQDIRNDVG |
| ZY0EQD-E02; CLDR2 | SEQ ID NO: 11: AASRLQS |
| ZY0EQD-E02; CLDR3 | SEQ ID NO: 12: LQHNSYPRT |
| ZY0EOB-F05; CHDR1 | SEQ ID NO: 13: SGGYYWS |
| ZY0EOB-F05; CHDR2 | SEQ ID NO: 14: NIYYSGSTYYNPSLKS |
| ZY0EOB-F05; CHDR3 | SEQ ID NO: 15: EKALATVTPSGYENYY |
| | TVDV |
| ZY0EOB-F05; CLDR1 | SEQ ID NO: 16: WASQGISSYLA |
| ZY0EOB-F05; CLDR2 | SEQ ID NO: 17: AASTLQS |
| ZY0EOB-F05; CLDR3 | SEQ ID NO: 18: QHLNSYPLT |
| ZY0EO5-E07; CHDR1 | SEQ ID NO: 19: SGGYYWS |
| ZY0EO5-E07; CHDR2 | SEQ ID NO: 20: NIYYSGSTYYNPSLKS |
| ZY0EO5-E07; CHDR3 | SEQ ID NO: 21: EKALASVIPSGYENYY |
| | VVDV |
| ZY0EO5-E07; CLDR1 | SEQ ID NO: 22: WASQGIAGYLA |
| ZY0EO5-E07; CLDR2 | SEQ ID NO: 23: AASTLQS |
| ZY0EO5-E07; CLDR3 | SEQ ID NO: 24: QHLNSYPLT |
| ZY0EP0-C07; CHDR1 | SEQ ID NO: 25: DYYMS |
| ZY0EP0-C07; CHDR2 | SEQ ID NO: 26: YISSSGSTIYYTDSVKG |
| ZY0EP0-C07; CHDR3 | SEQ ID NO: 27: DGVGFDY |
| ZY0EP0-C07; CLDR1 | SEQ ID NO: 28: RASQSVSSSYLA |
| ZY0EP0-C07; CLDR2 | SEQ ID NO: 29: AASSRAT |
| ZY0EP0-C07; CLDR3 | SEQ ID NO: 30: QQYGSSPLYT |

SEQ ID NO: 31 (ZY0EPQ-E02, variable heavy chain)
QVQLQQWGAGLLKPSETLSLTCAVYGGSFSGYYWNWIRQPPGKGLEWIGEINHSGSTNYNPS
LKSRVTILVDTSKNQFSLKLSSVTAADTAVYYCARNLYNWNLDSWGQGTLVTVSS
SEQ ID NO: 32 (ZY0EPQ-E02, variable light chain)
DIQMTQSPSSLSASVGDRVTITCRASQGIRNDLGWYQQKPGRAPKRLIYVASSLQSGVPSRFS
GSGSGTEFTLTISSLQPEDFATYYCLQHNSYPRTFGQGTKVEIK
SEQ ID NO: 33 (ZY0EQD-E02, variable heavy chain, e.g., pre-germlining)
QVQLQQWGAGLLKPSETLSLTCAVYGGSFSGYYWNWIRQPPGKGLEWIGEINHSGSTSYNPS
LKSRVTISVDTSKNQFSLKLSSVTAADTAVYYCARVLYNWNVDSWGQGTLVTVSS
SEQ ID NO: 34 (ZY0EQD-E02, variable light chain)
DIQMTQSPSSLSASVGDRVTITCRASQDIRNDVGWYQQKPGKAPKRLIYAASRLQSGVPSRFS
GSGSGTEFTLTISSLQPEDFATYYCLQHNSYPRTFGQGTKVEIK
SEQ ID NO: 35 (ZY0EOB-F05, variable heavy chain)
QVQLQESGPGLVKPSQTLSLTCTVSDGSISSGGYYWSWIRQHPGKGLEWIGNIYYSGSTYYNP
SLKSRVTISVDTSKNQFSLKLNSVTAADTAVYYCATEKALATVTPSGYENYYTVDVWGQGTTVT
VSS
SEQ ID NO: 36 (ZY0EOB-F05, variable light chain)
DIQLTQSPSFLSASVGDRVTITCWASQGISSYLAWYQQKPGKAPKLLIYAASTLQSGVPSRFSG
SGSGTEFTLTISSLQPEDFATYYCQHLNSYPLTFGGGTKVEIK
SEQ ID NO: 37 (ZY0EO5-E07, variable heavy chain)
QVQLQESGPGLVKPSQTLSLTCTVSGGSISSGGYYWSWIRQHPGKGLEWIGNIYYSGSTYYNP
SLKSRVTISVDTSKNQFSLKLSSVTAADTAVYYCAREKALASVIPSGYENYYVVDVWGQGTTVT
VSS
SEQ ID NO: 38 (ZY0EO5-E07, variable light chain)
DIQLTQSPSFLSASVGGRVTITCWASQGIAGYLAWYQQKPGKAPKLLIYAASTLQSGVPSRFSG
SGSGTEFTLTISSLQPEDFATYYCQHLNSYPLTFGGGTKVEIK
SEQ ID NO: 39: (ZY0EP0-C07, variable heavy chain)
QVQLVESGGVLVKPGGSLRLSCAASGFTLSDYYMSWIRQAPGMGLEWSYISSSGSTIYYTDS
VKGRFTISRDSAKNSLYLQMNSLRAEDTAVYYCARDGVGFDYWGQGTLVTVSS
SEQ ID NO: 40 (ZY0EP0-C07, variable light chain)
EIVLTQSPGTLSLFPGERATLSCRASQSVSSSYLAWYQQKPGQSPRLLIYAASSRATGIPDRFS
GSGSGTDFTLTISRLEPEDFAVYYCQQYGSSPLYTFGQGTKLEIK
SEQ ID NO: 41 (Maia heavy chain constant region, cysteine insertion underlined):
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSCVFLF
PPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 42 (light chain constant region)
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 43 (ZY0EQD-E02, variable heavy chain, e.g., pre-germlining, e.g., variant of
SEQ ID NO: 33/SEQ ID NO: 45)
QVQLQQWGAGLLKPSETLSLTCTVYGGSFSGYYWNWIRQPPGRGLEWIGEINHSGSTSYNPS
LKSRITISIDTSKNQFSLKLSSVTAADTAVYYCARVLYNWNVDSWGQGTLVTVSS
SEQ ID NO: 44 (ZY0EQD-E02, light chain):
DIQMTQSPSSLSASVGDRVTITCRASQDIRNDVGWYQQKPGKAPKRLIYAASRLQSGVPSRFS
GSGSGTEFTLTISSLQPEDFATYYCLQHNSYPRTFGQGTKVEIKRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC
SEQ ID NO: 45 (EQD-E02_GL, variable heavy chain, GL = germlined)
QVQLQQWGAGLLKPSETLSLACTVYGGSFSGYYWNWIRQPPGKGLEWIGEINHSGSTSYNPS
LKSRVTISVDTSKNQFSLKLSSVTAADTAVYYCARVLYNWNVDSWGQGTLVTVSS
SEQ ID NO: 46 (EQD-E02-GLY, variable heavy chain, GLY = germlined with a Y
substitution)
QVQLQQWGAGLLKPSETLSLACTVYGGSFSGYYWNWIRQPPGKGLEWIGEIYHSGSTSYNPS
LKSRVTISVDTSKNQFSLKLSSVTAADTAVYYCARVLYNWNVDSWGQGTLVTVSS
SEQ ID NO: 47 (EQD-E02-GLQ, variable heavy chain, GLQ = germlined with a Q
substitution)
QVQLQQWGAGLLKPSETLSLACTVYGGSFSGYYWNWIRQPPGKGLEWIGEIQHSGSTSYNPS
LKSRVTISVDTSKNQFSLKLSSVTAADTAVYYCARVLYNWNVDSWGQGTLVTVSS
SEQ ID NO: 48 (E02-GL-Maia-heavy chain, cysteine insertion underlined)
QVQLQQWGAGLLKPSETLSLACTVYGGSFSGYYWNWIRQPPGKGLEWIGEINHSGSTSYNPS
LKSRVTISVDTSKNQFSLKLSSVTAADTAVYYCARVLYNWNVDSWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSCVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 49 (E02-GLY-Maia-heavy chain, GLY = germlined with a Y substitution)
QVQLQQWGAGLLKPSETLSLACTVYGGSFSGYYWNWIRQPPGKGLEWIGEIYHSGSTSYNPS
LKSRVTISVDTSKNQFSLKLSSVTAADTAVYYCARVLYNWNVDSWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSCVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 50 (E02-GLQ-Maia-heavy chain, GLQ = germlined with a Q substitution)
QVQLQQWGAGLLKPSETLSLACTVYGGSFSGYYWNWIRQPPGKGLEWIGEIQHSGSTSYNPS
LKSRVTISVDTSKNQFSLKLSSVTAADTAVYYCARVLYNWNVDSWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSCVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 51 (E02-GL-WT-heavy chain)
QVQLQQWGAGLLKPSETLSLACTVYGGSFSGYYWNWIRQPPGKGLEWIGEINHSGSTSYNPS
LKSRVTISVDTSKNQFSLKLSSVTAADTAVYYCARVLYNWNVDSWGQGTLVTVSSASTKGPSV
FPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 52 (heavy chain constant region)
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSCVFLF
PPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 53 (Human B7H4 nucleic acid sequence, including 5′ and 3′ UTR)
GCCACCatggcttccctggggcagatcctcttctggagcataattagcatcatcattattctggctggagcaattgcactcatcattgg
ctttggtatttcagggagacactccatcacagtcactactgtcgcctcagctgggaacattggggaggatggaatcctgagctgcactttt
gaacctgacatcaaactttctgatatcgtgatacaatggctgaaggaaggtgttttaggcttggtccatgagttcaaagaaggcaaaga
tgagctgtcggagcaggatgaaatgttcagaggccggacagcagtgtttgctgatcaagtgatagttggcaatgcctctttgcggctga
aaaacgtgcaactcacagatgctggcacctacaaatgttatatcatcacttctaaaggcaaggggaatgctaaccttgagtataaaac
tggagccttcagcatgccggaagtgaatgtggactataatgccagctcagagaccttgcggtgtgaggctccccgatggttcccccag
cccacagtggtctgggcatcccaagttgaccagggagccaacttctcggaagtctccaataccagctttgagctgaactctgagaatgt
gaccatgaaggttgtgtctgtgctctacaatgttacgatcaacaacacatactcctgtatgattgaaaatgacattgccaaagcaacag
gggatatcaaagtgacagaatcggagatcaaaaggcggagtcacctacagctgctaaactcaaaggcttctctgtgtgtctcttctttctt
tgccatcagctgggcacttctgcctctcagcccttacctgatgctaaaaTAATAA
SEQ ID NO: 54 (Human B7H4 nucleic acid sequence, coding sequence)
atggcttccctggggcagatcctcttctggagcataattagcatcatcattattctggctggagcaattgcactcatcattggctttggtatttc
agggagacactccatcacagtcactactgtcgcctcagctgggaacattggggaggatggaatcctgagctgcacttttgaacctgac
atcaaactttctgatatcgtgatacaatggctgaaggaaggtgttttaggcttggtccatgagttcaaagaaggcaaagatgagctgtcg
gagcaggatgaaatgttcagaggccggacagcagtgtttgctgatcaagtgatagttggcaatgcctctttgcggctgaaaaacgtgc
aactcacagatgctggcacctacaaatgttatatcatcacttctaaaggcaaggggaatgctaaccttgagtataaaactggagccttc
agcatgccggaagtgaatgtggactataatgccagctcagagaccttgcggtgtgaggctccccgatggttcccccagcccacagtg
gtctgggcatcccaagttgaccagggagccaacttctcggaagtctccaataccagctttgagctgaactctgagaatgtgaccatga
aggttgtgtctgtgctctacaatgttacgatcaacaacacatactcctgtatgattgaaaatgacattgccaaagcaacaggggatatca
aagtgacagaatcggagatcaaaaggcggagtcacctacagctgctaaactcaaaggcttctctgtgtgtctcttctttctttgccatcag
ctgggcacttctgcctctcagcccttacctgatgctaaaa
SEQ ID NO: 55 (Human B7H4 polypeptide sequence; UniProt Accession No.: Q7Z7D3)
MASLGQILFWSIISIIIILAGAIALIIGFGISGRHSITVTTVASAGNIGEDGILSCTFEPDIKLSDIVIQWL
KEGVLGLVHEFKEGKDELSEQDEMFRGRTAVFADQVIVGNASLRLKNVQLTDAGTYKCYIITSK
GKGNANLEYKTGAFSMPEVNVDYNASSETLRCEAPRWFPQPTVVWASQVDQGANFSEVSNT
SFELNSENVTMKVVSVLYNVTINNTYSCMIENDIAKATGDIKVTESEIKRRSHLQLLNSKASLCVS
SFFAISWALLPLSPYLMLK

What is claimed is:

1. A method of treating cancer in a human subject in need thereof, comprising administering to the human subject:
A) an antibody-drug conjugate (ADC) comprising:
i. an antibody or antigen binding fragment thereof which binds to a B7-H4 polypeptide, comprising:
a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker;
iii. a cytotoxic agent; and
B) an additional agent, wherein the additional agent is a PARP1 inhibitor or an ATR inhibitor.

2. The method of claim 1, wherein the additional agent is AZD5305 or a pharmaceutically acceptable salt thereof.

3. (canceled)

4. The method of claim 1, wherein the cancer comprises a cancer cell which expresses B7-H4 and wherein the cancer further comprises a cancer cell that does not express B7-H4.

5. (canceled)

6. The method of claim 1, wherein the cancer is selected from ovarian cancer, breast cancer, pancreatic cancer, prostate cancer, hematological cancer, endometrial cancer, cholangiocarcinoma, NSCLC (squamous and/or adenocarcinoma), gastrointestinal cancer such as gastric cancer and colorectal cancer, and lung cancer.

7. The method of claim 1, wherein the cancer is a breast cancer selected from hormone receptor-positive (HR+) breast cancer, human epidermal growth factor receptor 2 positive (HER2+) breast cancer, and triple negative breast cancer (TNBC).

8. The method of claim 1, wherein the cancer is homologous recombination deficient (HRD) cancer, and wherein the cancer comprises one or more cells having a mutation in an HRD gene selected from BRCA1, BRCA2, ATM, BRIP1, BARD1, CDK12, CHEK1, CHEK2, FANCL, PALB2, PPP2R2A, RAD51B, RAD51C, RAD51D, and RAD54L.

9-10. (canceled)

11. The method of claim 1, wherein the antibody or antigen binding fragment thereof comprises:
i. a variable heavy (VH) chain and a variable light (VL) chain comprising the amino acid sequence of SEQ ID NO: 45 and SEQ ID NO: 34, respectively, or a functional variant thereof;
ii. a variable heavy (VH) chain and a variable light (VL) chain comprising the amino acid sequence of SEQ ID NO: 33 and SEQ ID NO: 34, respectively, or a functional variant thereof;
iii. a variable heavy (VH) chain and a variable light (VL) chain comprising the amino acid sequence of SEQ ID NO: 43 and SEQ ID NO: 34, respectively, or a functional variant thereof;
iv. a variable heavy (VH) chain and a variable light (VL) chain comprising the amino acid sequence of SEQ ID NO: 46 and SEQ ID NO: 34, respectively, or a functional variant thereof;
v. a variable heavy (VH) chain and a variable light (VL) chain comprising the amino acid sequence of SEQ ID NO: 47 and SEQ ID NO: 34, respectively, or a functional variant thereof;
vi. a VH chain and a VL chain comprising the amino acid sequence of SEQ ID NO: 31, and SEQ ID NO: 32, respectively, or a functional variant thereof;
vii. a VH chain and a VL chain comprising the amino acid sequence of SEQ ID NO: 35 and SEQ ID NO: 36, respectively, or a functional variant thereof,
viii. a VH chain and a VL chain comprising the amino acid sequence of SEQ ID NO: 37 and SEQ ID NO: 38, respectively, or a functional variant thereof, or
ix, a VH chain and a VL chain comprising the amino acid sequence of SEQ ID NO: 39 and SEQ ID NO: 40, respectively, or a functional variant thereof.

12. The method of claim 1, wherein the antibody or antigen binding fragment thereof comprises:
i. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

13. The method of claim 12, wherein the antibody or antigen binding fragment thereof comprises:
i. a VH chain and a VL chain comprising the amino acid sequence of SEQ ID NO: 45 and SEQ ID NO: 34, respectively, or a functional variant thereof.

14-15. (canceled)

16. The method of claim 13, wherein the antibody or antigen binding fragment thereof comprises a heavy chain constant region comprising the amino acid sequence of SEQ ID NO: 52, and a light chain constant region comprising the amino acid sequence of SEQ ID NO: 42.

17. (canceled)

18. The method of claim 13, wherein the antibody or antigen binding fragment thereof comprises a heavy chain comprising the amino acid sequence of SEQ ID NO: 51; and a light chain comprising the amino acid sequence of SEQ ID NO: 44.

19-20. (canceled)

21. The method of claim 1, wherein the antibody or antigen binding fragment thereof is a humanized monoclonal antibody.

22. The method of claim 1, wherein the cleavable linker is an mp-PEG8-val-ala linker.

23. The method of claim 1, wherein the cytotoxic agent is a topoisomerase inhibitor.

24. The method of claim 23, wherein the topoisomerase inhibitor is a compound of Formula A*

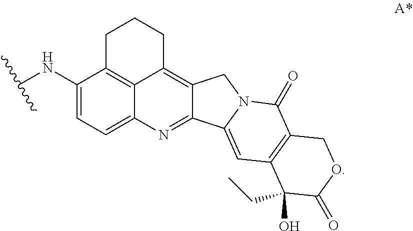 [SMILES: *Nc1ccc2nc3c(c4c2c1CCC4)Cn1c-3cc2c(c1=O)COC(=O)[C@]2(O)CC]

25. The method of claim 24, wherein the ii) cleavable linker and iii) cytotoxic agent are together selected from the following compounds:

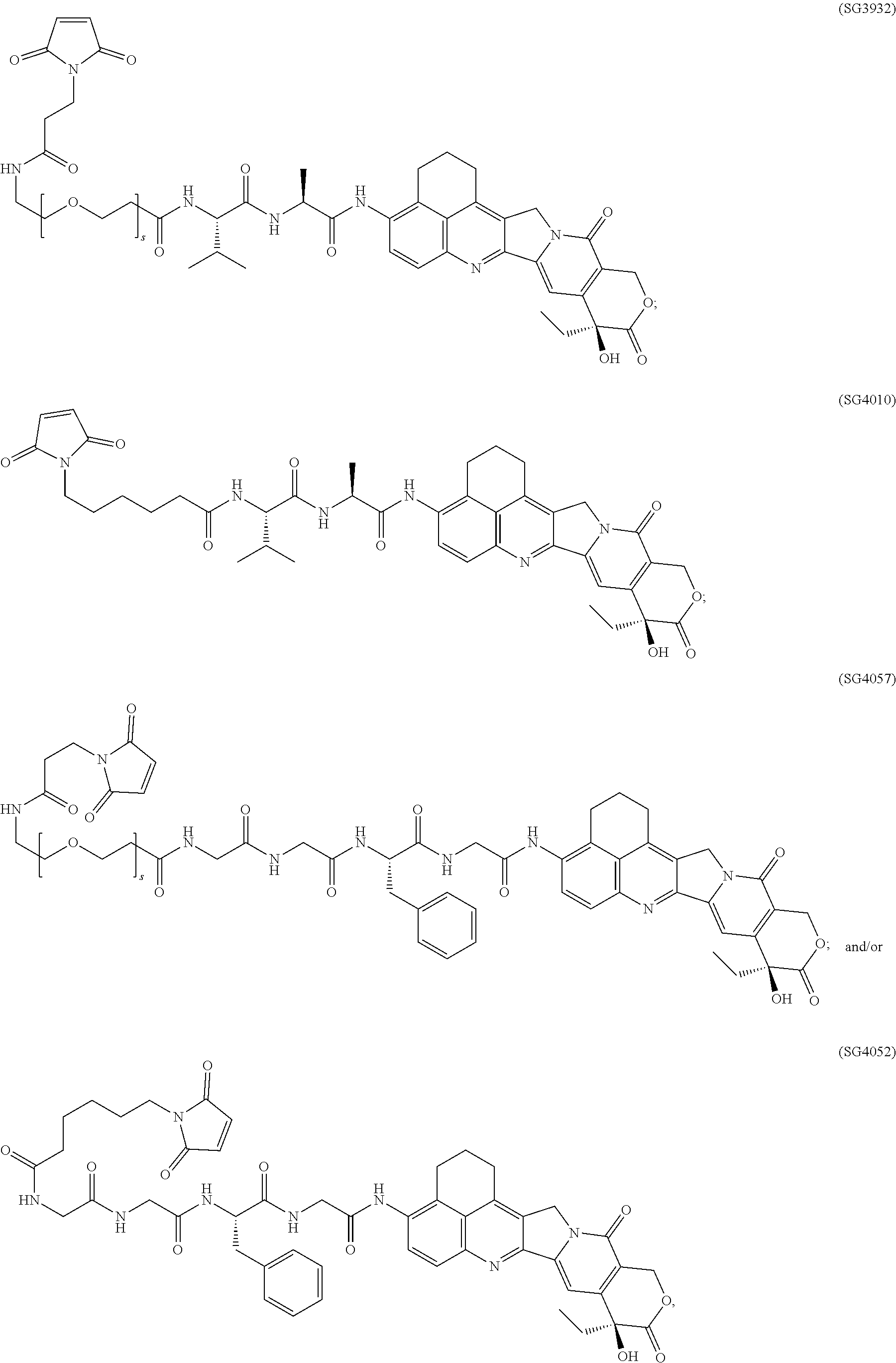

26. (canceled)

27. The method of claim 1, wherein the ADC has a drug to antibody ratio (DAR) of between about 1 and about 8.

28. (canceled)

29. A kit comprising:
A) an antibody-drug conjugate (ADC) comprising:
i. an antibody or antigen binding fragment thereof which binds to a B7-H4 polypeptide comprising:
a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof,
b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
ii. a cleavable linker;
iii. a cytotoxic agent; and
B) an additional agent, wherein the additional agent is a PARP1 inhibitor or an ATR inhibitor.

30-45. (canceled)

46. A method of treating cancer in a human subject in need thereof, comprising administering to the human subject:
A) an antibody-drug conjugate (ADC) comprising:
i. an antibody or antigen binding fragment thereof which binds to a B7-H4 polypeptide, comprising:
a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; and
ii. a cleavable linker and cytotoxic agent conjugated to the antibody or antigen binding fragment thereof having the formula: 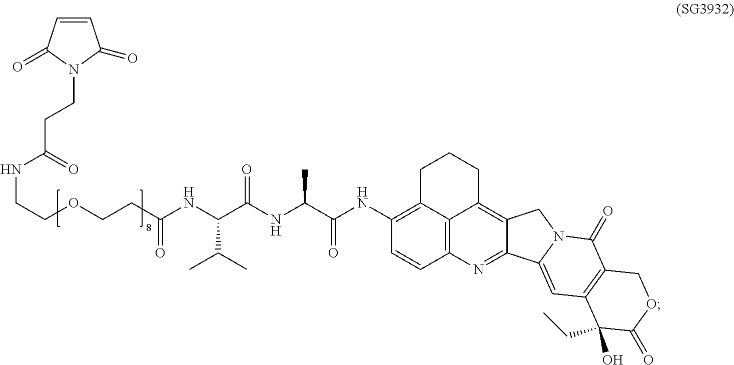
and
B) AZD5305 or a pharmaceutically acceptable salt thereof.

47. A method of treating cancer in a human subject in need thereof, comprising administering to the human subject:
A) an antibody-drug conjugate (ADC) comprising:
i. an antibody or antigen binding fragment thereof which binds to a B7-H4 polypeptide comprising:
a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
b) a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof,
c) a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
d) a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
e) a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; and
ii. a cleavable linker and cytotoxic agent conjugated to the antibody or antigen binding fragment thereof having the formula: 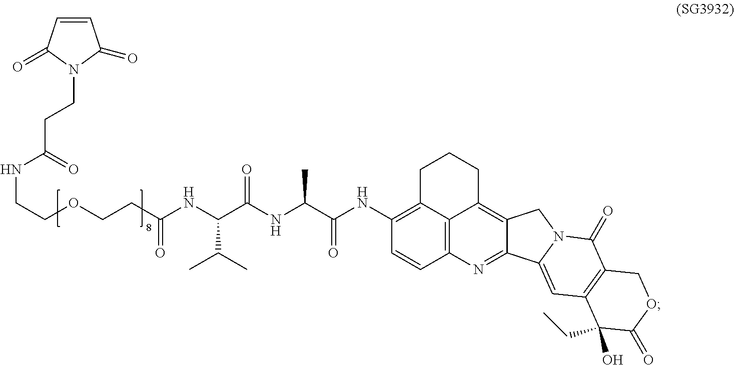
and
B) AZD6738 or a pharmaceutically acceptable salt thereof.

48-54. (canceled)
